SEQUENCE LISTING (2 sequences)

SEQ ID NO: 1 - protein, 27 aa, Artificial Sequence
  A synthetic peptide
GALFLGFLGAAGSTMGAWSQPKKKRKV

SEQ ID NO: 2 - protein, 27 aa, Artificial Sequence
  A synthetic peptide
GALFLGFLGAAGSTMGAWSQPKSKRKV

(19) United States
(12) Patent Application Publication
(10) Pub. No.: US 2011/0229457 A1
(43) Pub. Date: Sep. 22, 2011

(54) INJECTABLE DRUG DELIVERY SYSTEM

(75) Inventors: Timothy M. Kloke, Victoria, MN (US); Joram Slager, St. Louis Park, MN (US); Laxma Gundlapally Reddy, Shoreview, MN (US); David E. Babcock, St. Louis Park, MN (US)

(73) Assignee: SurModics, Inc., Eden Prairie, MN (US)

(21) Appl. No.: 13/046,547

(22) Filed: Mar. 11, 2011

Related U.S. Application Data
(60) Provisional application No. 61/313,666, filed on Mar. 12, 2010.

(51) Int. Cl. A61K 39/395; A61K 47/36; A61K 38/02; A61K 38/43; A61K 31/713; A61P 25/28; A61P 25/16; A61P 25/00; A61P 27/02; A61P 27/06; A61P 31/20; A61P 31/14; A61P 19/02; A61K 31/7088; A61K 31/7052

(52) U.S. Cl. 424/130.1; 514/777; 514/1.1; 424/94.1; 514/44 A; 514/778; 514/44 R; 514/43

(57) ABSTRACT

The invention provides a formulation that includes a biocompatible solvent system, a biodegradable polymer that is substantially soluble in the biocompatible solvent system, and an active pharmaceutical ingredient that is substantially insoluble in the biocompatible solvent system. The formulation can form a drug-eluting implant, when injected into mammalian tissue. The solvent system and the biodegradable polymer can be selected so that the implant provides extended, delayed, controlled and/or modified release of the active pharmaceutical ingredient, for example, over the course of days, weeks or months.

### RELATED APPLICATIONS

This application claims priority to U.S. Ser. No. 61/313,666 filed on Mar. 12, 2010, which is incorporated by reference herein in its entirety.

### BACKGROUND

What is needed are injectable formulations that can effectively administer, to a mammal, an active pharmaceutical ingredient (API) over an extended period of time, in a controlled, extended, delayed and/or modified manner, with little or no initial drug burst.

### SUMMARY

The present invention provides a formulation that includes: (a) a biocompatible solvent system; (b) a biodegradable polymer that is substantially soluble in the biocompatible solvent system; and (c) an active pharmaceutical ingredient (API) that is substantially insoluble in the biocompatible solvent system.

The present invention also provides a formulation that includes: (a) a biocompatible solvent system; (b) a biodegradable polymer that is substantially soluble in the biocompatible solvent system, and (c) an active pharmaceutical ingredient (API) that is substantially insoluble in the biocompatible solvent system. The biodegradable polymer includes a polysaccharide that includes a unit of formula (I):

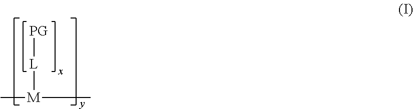

wherein: each M is independently a monosaccharide unit; each L is independently a suitable linking group or a direct bond; each PG is independently a pendent group; each x is independently 0 to about 3, such that when x is 0, the bond between L and M is absent; and y is 3 to about 10,000.

The polysaccharide that includes the unit of formula (I) can be, for example, a compound of formula (II):

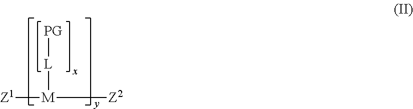

wherein: each M is a monosaccharide unit; each L is a suitable linking group, or is a direct bond; each PG is a pendent group; each x is independently 0 to about 3, such that when x is 0, the bond between L and M is absent; y is about 3 to about 5,000; Z¹ and Z² are each independently hydrogen, OR¹, OC(═O)R¹, CH₂OR¹, SiR¹ or CH₂OC(═O)R¹; each R¹ is independently hydrogen, alkyl, cycloalkyl, cycloalkyl alkyl, aryl, aryl alkyl, heterocyclyl or heteroaryl; each alkyl, cycloalkyl, aryl, heterocycle and heteroaryl is optionally substituted; and each alkyl, cycloalkyl and heterocycle is optionally partially unsaturated.

The present invention also provides for a composition that includes: (a) a solvent system that includes one or more of ethyl heptanoate, glycofural, and benzyl benzoate; (b) a substituted maltodextrin having an average MW of about 50 kDa to about 350 kDa; and (c) an active pharmaceutical ingredient comprising one or more of a PEGylated protein, PEGylated aptamer, enzyme, blood clotting factor, cytokine, hormone, a growth factor, an antibody and siRNA. The substituted maltodextrin includes a plurality of (C₂-C₇)alkanoate pendant groups. The substituted maltodextrin also has a degree of substitution of about 0.5 to about 2. The substituted maltodextrin also has a solubility of at least about 50 g/L in the solvent system at 25° C. and 1 atm. The solubility of the active pharmaceutical ingredient is less than about 250 mg/L in the solvent system at 25° C. and 1 atm and the solubility of the active pharmaceutical ingredient is greater than about 25 g/L in water at 25° C. and 1 atm. The active pharmaceutical ingredient is suspended in the formulation and is present in about 0.1 wt. % to about 30 wt. % of the formulation.

The present invention also provides for a method that includes administering to a mammal a formulation described herein. The formulation can be administered as an injectable formulation, for example, via an ocular administration or subcutaneously. Subsequent to the administration, an implant can be formed in vivo in the mammal, and the API can be locally or systemically delivered. The formulation can be administered to the mammal about once a day to about once per 6 months, to effectively treat at least one of the following diseases or disorders: age-related macular degeneration (wet and dry), diabetic macular edema (DME), glaucoma, keratoconjunctivitis sicca (KCS) or dry eye syndrome, multiple sclerosis, rheumatoid arthritis, Alzheimer's disease, Parkinson's disease, amyotrophic lateral sclerosis (ALS), Hepatitis B and C, and systemic lupus erythematosus. The solid biodegradable implant that is formed in vivo can biodegrade within about 1 year after the formulation is administered.

The present invention also provides for the formulations and/or compositions described herein, for the treatment of a disease. More specifically, the present invention also provides for the formulations and/or compositions described herein, for the treatment of at least one of the following diseases or disorders: age-related macular degeneration (wet and dry), diabetic macular edema (DME), glaucoma, keratoconjunctivitis sicca (KCS) or dry eye syndrome, multiple sclerosis, rheumatoid arthritis, Alzheimer's disease, Parkinson's disease, amyotrophic lateral sclerosis (ALS), Hepatitis B and C, and systemic lupus erythematosus.

### BRIEF DESCRIPTION OF THE DRAWINGS

The following drawings form part of the specification and are included to further demonstrate certain embodiments or various aspects of the invention. In some instances, embodiments can be best understood by referring to the accompanying drawings in combination with the detailed description presented herein. The description and accompanying drawings may highlight a certain specific example, or a certain aspect of the invention, however, one skilled in the art will understand that portions of the example or aspect may be used in combination with other examples or aspects of the invention.

FIG. 1 illustrates a comparison of polymer versus no polymer in an experiment showing a significantly reduced burst using a composition according to Example 1. Cumulative in vitro HRP release in PBS, 37° C.

FIG. 2 illustrates the effect of biodegradable polysaccharides polymer type on elution control, according to Example 2. Cumulative in vitro rabbit Fab release in PBS at 37° C.; Fab load in 50 μL depot=2.1 mg.

FIG. 3 illustrates a comparison of cumulative release for biodegradable polysaccharides versus poly(DL-lactide-co-glycolide) according to Example 2. Cumulative in vitro HRP release in PBS at 37° C.

FIG. 4 illustrates the effect of biodegradable polysaccharides concentration, according to Example 3. Cumulative in vitro rabbit Fab release in PBS at 37° C.; Fab load in 50 μL depot=2.1 mg.

FIG. 5 illustrates the effect of solvent on elution, according to Example 4. Cumulative in vitro rabbit Fab release in PBS at 37° C.; Fab load in 50 μL depot=2.1 mg.

FIG. 6 illustrates the effect of protein load on elution according to Example 5. Cumulative in vitro HRP release in PBS at 37° C.

FIG. 7 illustrates cumulative release profiles of Fab from biodegradable polysaccharides organogels in aliphatic esters, formulated at room temperature, wherein the organogel formulation is injected into PBS solution according to experiments described in Example 6.

FIG. 8 illustrates cumulative release profiles of Fab from biodegradable polysaccharides organogels in aliphatic esters at 150 or 200 mg/mL, wherein the organogel formulation is injected into a PBS solution, according to experiments described in Example 8.

FIG. 9 illustrates Fab release from Glu2-hex-1.6 gelled in ethyl heptanoate at 300 mg/mL, according to experiments described in Example 8.

FIG. 10 illustrates cumulative release profiles of Fab from biodegradable polysaccharides organogels, in ethyl hexanoate at different concentrations, wherein a PBS solution is put on top of the organogel formulation.

FIG. 11 illustrates cumulative release profiles of Fab from biodegradable polysaccharides organogels formulated at 55° C. in various solvents at concentrations of 150-300 mg/mL, wherein the polymer solution was heated and added to Fab particles; the formulation was then mixed and allowed to cool, followed by adding PBS to the formulation, according to experiments described in Example 9.

### DETAILED DESCRIPTION

The present invention is directed to a composition that includes a biocompatible solvent system, a biodegradable polymer, and an active pharmaceutical ingredient (API). The biodegradable polymer is substantially soluble in the biocompatible solvent system, while the API is substantially insoluble in the biocompatible solvent system. The composition can be a homogenous suspension, such that the API is homogeneously dispersed (i.e., undissolved, unsolubilized and/or suspended) throughout the composition. As such, upon forming an implant in vivo, the API can be homogeneously suspended throughout the implant. In some embodiments, the solid biodegradable implant is monolithic.

The present invention is also directed to a method of systemically or locally administering an API to a subject by administering a composition that includes a biocompatible solvent system, a biodegradable polymer, and an API. The composition, upon administration, can form an implant in vivo (i.e., upon contact with body fluids).

In one embodiment, a viscous gel can be formed from the polymer and the solvent. In another embodiment, a viscous gel can be formed upon cooling the polymer and the solvent. In other embodiments, the polymer, solvent and API forms a composition that is not gelled.

The composition can have a viscosity of, for example, less than 5000 cP at room temperature. Although viscous, the composition can be formulated as an injectable delivery system, through a needle. The delivery system can be, for example, an injectable ocular delivery system, an injectable subcutaneous delivery system or an injectable parenteral delivery system. As such, the composition can be flowable and can be formulated for injection through, e.g., a 25 gauge needle, or a higher gauge needle (e.g., a 30 gauge needle). The volume of the delivery system can be suitable for injection into a mammal, such as a human. For example, suitable injection volumes can be about 10 μL to about 100 μL, or about 0.01 mL to about 2.0 mL. The injectable delivery system is thus suitable for forming an implant (e.g., a controlled-release implant) in vivo.

By appropriate choice of solvent, water migration from the aqueous environment surrounding the implant is restricted, and active pharmaceutical ingredient (API) is released to the subject over a period of time, thus providing for delivery of the API with a controlled burst (e.g., little or no initial burst) of API, and sustained release thereafter. The implant formed is bioerodible, such that the implant does not have to be surgically removed after the API is depleted from the implant.

Water uptake and burst can be controlled by using polymer-solvent compositions wherein the solvent is substantially immiscible in water, so as to control the rate of water migration into the polymer implant and ultimately control the burst of API and the sustained delivery of the API. Generally, the compositions will form an implant upon exposure to an aqueous environment, such as mammalian tissue. Furthermore, while the polymer gel implant will slowly harden when subjected to an aqueous environment, the hardened implant can maintain a rubbery (non-rigid) quality as a result of a glass transition temperature of about 37° C., or less.

Because implants formed from the compositions described herein can be formed from viscous compositions, administration of the viscous composition is not limited to injection, although that mode of delivery may often be preferred. Where the implant will be administered as a leave-behind product, it can be formed to fit into a body cavity existing after completion of surgery or it can be applied as a flowable gel by brushing the gel onto residual tissue or bone. Such applications may permit loading of APIs in the gel above concentrations typically present with injectable compositions.

The API can be incorporated in the form of particles. In some embodiments, the particles can be suspended in the formulation, thereby forming a suspension. The particles can have an average particle size of about 0.1 to about 100 microns, from about 1 to about 25 microns, from about 1 to about 20 microns, from about 0.1 to about 10 microns, or from about 2 to about 10 microns. In some embodiments, the particles can have an average particle size of at least about 0.1 or about 1 microns and less than about 25, 20, 15, or 10 microns. For instance, particles having an average particle size of about 5 microns have been produced by spray drying or freeze drying an aqueous mixture containing 50% sucrose and 50% lysozyme (on a dry weight basis) and mixtures of hGH (e.g., 5-30% or 10-20%) and zinc acetate (e.g., 10-40 mM or 15-30 mM). Such particles have been used in certain of the examples illustrated in the figures herein. Conventional lyophilization processes can also be used to form particles of beneficial agents of varying sizes using appropriate freezing and drying cycles. As such, the API can be in the form of a spray-dried protein (e.g., fab or IgG).

To form a suspension of particles of the API in the viscous composition (e.g., gelled composition), any conventional low shear device can be used, such as a Ross double planetary mixer at ambient conditions. In this manner, efficient distribution of the API can be achieved substantially without degrading the API.

When the composition is intended for administration by injection, an implant will form in vivo, upon contact with body fluids. When the composition is intended for administration as an implant, the implant can be pre-formed and subsequently introduced within the body of a patient. Either way, an effective amount of the API can be released by diffusion, erosion, absorption, degradation, or a combination thereof, as the solid implant biodegrades in the patient.

The nature and amount of solvent, polymer and API can be selected such that the desired duration of administration is achieved. For example, the formulation can be administered to release an effective amount of API over a suitable period of time, for example, of about once a day to about once per 12 months, about once a day to about once per 6 months, about once a day to about once per 3 months, about once a day to about once per 1 month, or about once a day to about once per 7 days.

The nature and amount of solvent, polymer and API can be selected such that the desired composition or formulation will have an acceptable chemical and/or physical stability. Such stability can be, for example, for up to 6 months, up to 1 year, up to 2 years or up to 5 years. Additionally, the nature and amount of solvent, polymer and API can be selected such that the resulting implant formed will have an acceptable chemical and/or physical stability. Such stability can be, for example, from about 1 day to about 2 years, from 1 day to about 1 year, from 1 day to about 6 months, from about 1 day to about 3 months or from about 1 day to about 1 month.

### Definitions

Unless stated otherwise, the following terms and phrases as used herein are intended to have the following meanings:

When tradenames are used, applicants intend to independently include the tradename product and the active pharmaceutical ingredient(s) of the tradename product.

The term “injectable formulation” refers to a pharmaceutical composition suitable for injection into the tissues of a living organism.

The term “biocompatible solvent” refers to a liquid material that can be emplaced within living tissue of an organism without causing significant damage to the tissue or organism.

The term “biodegradable polymer” refers to a polymeric material, as is well known in the art, that when emplaced within living tissue of an organism undergoes chemical breakdown.

The term “active pharmaceutical ingredient (API)” refers to a therapeutic, medicinal substance, such as is commonly termed a “drug” or a “medicament” suitable for administration for medical treatment of a malcondition in a living organism, such as a human being. An active pharmaceutical ingredient can be, but is not limited to, a macromolecule, a protein, a peptide, a gene, a polynucleotide or analog thereof, a nucleotide, a biological agent, a small molecule, or a complex thereof.

The term “insolubility” is a standard term used in the art, and meaning 1 part solute per 10,000 parts or greater solvent. (See, for example, Remington: The Science and Practice of Pharmacy, 20th ed. (2000), Lippincott Williams & Wilkins, Baltimore Md.).

The term “substantially soluble” refers to a property of a substance that dissolves completely or almost completely in a liquid material (e.g., at least 1,000 parts solute per 10,000 parts solvent). For example, a biodegradable polymer can be substantially soluble in a biocompatible solvent, such that the polymer solution in the solvent can be injected into living tissue of an organism without causing significant damage to the tissue or organism. In specific embodiments, at least about 98 wt. % of the substance completely dissolves in the liquid material at room temperature. In further specific embodiments, at least about 99 wt. % of the substance completely dissolves in the liquid material.

The term “substantially insoluble” refers to a substance that does not dissolve to any significant extent in a liquid material (e.g., 1 part solute per 10,000 parts or greater solvent). For example, an active pharmaceutical ingredient can be substantially insoluble in a biocompatible solvent in which a biodegradable polymer is substantially soluble. The active pharmaceutical ingredient can be suspended in the solution, e.g., in microparticulate or nanoparticulate form. In specific embodiments, less than about 5 wt. % of the substance (e.g., active pharmaceutical ingredient) completely dissolves in the liquid material at room temperature. In further specific embodiments, less than about 1 wt. % of the substance (e.g., active pharmaceutical ingredient) completely dissolves in the liquid material. In further specific embodiments, less than about 0.1 wt. % of the substance (e.g., active pharmaceutical ingredient) completely dissolves in the liquid material.

The term “hydrophilic” refers to a property wherein a substance, a molecule, or a domain of a substance or molecule has an affinity for water or aqueous fluids. A hydrophilic substance, molecule, or domain can dissolve, become deliquescent, or be wetted by water or the aqueous substance. A substance, molecule, or a domain thereof is hydrophilic when the energetics of the interaction between the substance, molecule, or domain and water or an aqueous fluid is favorable.

The term “macromolecule” refers to an organic molecule having a molecular weight of greater than about 2000 daltons. The term can refer to natural polymers such as proteins, polysaccharides and nucleic acids, or can refer to synthetic polymers such as polyesters.

The term “nucleotide” refers to a molecular entity composed of a nucleobase, sugar moiety, and phosphate group, or analogs thereof. Examples include the DNA nucleotides, i.e., adenine, guanine, cytosine, and thymidine, or the RNA nucleotide uracil, or synthetic analogs thereof. Examples of sugar moieties to which the nucleobases are covalently bonded include but are not limited to ribose and deoxyribose. Analogs of sugars can also be present; for example, halodeoxyribose analogs.

The term “biological agent” refers to a medicinally bioactive substance derived from a biological source, such as from an organism, a cell line, an isolated tissue, or the like.

The term “small molecule” refers to a molecular entity, often organic or organometallic, that is not a polymer, having medicinal activity. The molecular weight is typically less than about 2 kDa, and is often less than about 1 kDa. The term encompasses most medicinal compounds termed “drugs” other than protein or nucleic acids, although a small peptide or nucleic acid analog can be considered a small molecule within the meaning herein. Examples include anticancer drugs, antibiotics, anti-inflammatories, and other therapeutic substances. Small molecules can be derived synthetically, semi-synthetically (i.e., from naturally occurring precursors), or biologically.

The term “complex” refers to a molecular association, which can be non-covalent, between two molecular or atomic entities. For example, certain metals bind organic groups and are referred to as complexes, such as anticancer agent cisplatin. Or, certain macromolecules such as proteins can bind small molecule ligands, the product also being termed a complex. Complexes can also form between nucleic acid molecules, such as in DNA complementary nucleotide pairing and in association of DNA with RNA via complementary nucleotide pairing. A complex formed between DNA or messenger RNA and a small interfering RNA (siRNA) is another example of complementary nucleotide pairing.

The term “spray-dried protein” refers to a protein that has undergone a process of drying from a solution, which can be a water solution, wherein a relatively fine spray of the solution is subjected to conditions such as vacuum that serve to remove the liquid, e.g., water, and provide a finely powdered form of the protein.

Since all solvents, at least on a molecular level, will be soluble in water (i.e., miscible with water) to some very limited extent, the term “water immiscible” refers to a liquid that does not mix in all proportions with water. A water immiscible liquid can dissolve to some extent in water, but at some relative proportions of water and the liquid, phase separation occurs. In specific embodiments, for a water immiscible solvent, about 10 wt. % or less of the solvent is soluble in or is miscible with water. In another specific embodiments, for a water immiscible solvent, about 5 wt. % or less of the solvent is soluble in or is miscible with water. In another specific embodiments, for a water immiscible solvent, about 1 wt. % or less of the solvent is soluble in or is miscible with water. For the purposes of this invention, solubility values of solvent in water are considered to be determined at 20° C. Since it is generally recognized that solubility values as reported may not always be conducted at the same conditions, solubility limits described herein as percent by weight miscible or soluble with water as part of a range or upper limit may not be absolute.

Water miscibility can be determined experimentally as follows: Water (1-5 g) is placed in a tared clear container at a controlled temperature, about 20° C., and weighed, and a candidate solvent is added dropwise. The solution is swirled to observe phase separation. When the saturation point appears to be reached, as determined by observation of phase separation, the solution is allowed to stand overnight and is re-checked the following day. If the solution is still saturated, as determined by observation of phase separation, then the percent (w/w) of solvent added is determined. Otherwise more solvent is added and the process repeated. Solubility or miscibility is determined by dividing the total weight of solvent added by the final weight of the solvent/water mixture. When solvent mixtures are used, for example 20% triacetin and 80% benzyl benzoate, they are pre-mixed prior to adding to the water.

The term “miscible to dispersible in aqueous medium or body fluid” refers to a substance or mixture that interacts with aqueous medium, i.e., a liquid containing water, or with body fluid, e.g., blood, intercellular fluid, or the like, such that the solubility of the substance or mixture is either complete (miscible) or partially soluble (dispersible) in the fluid.

The term “immiscible to insoluble in aqueous medium or body fluid” refers to a substance or mixture that does not dissolve to any significant extent in aqueous media or body fluids, as described above.

The term “liquid” refers to a substance that is in physical form a mobile material having no long term order at around ambient and physiological temperature, i.e., a fluid but not a gas.

The term “ambient and physiological temperature” refers to the temperature range of about 15° C. to about 40° C. (e.g., 33.2-38.2° C. in normal and relatively healthy humans).

The term “aprotic solvent” refers to a liquid that does not contain exchangeable protons. Exchangeable protons can be found, e.g., on hydroxyl groups, so aprotic solvents do not include water, alcohols, or carboxylic acids. Examples of aprotic solvents are hydrocarbons, amides such as dimethylformamide, esters such as ethyl acetate, sulfoxides such as dimethylsulfoxide, and the like.

The term “miscible to dispersible in aqueous medium or bodily fluids” refers to a substance or mixture that either completely dissolves or uniformly distributes throughout an aqueous medium or body fluid.

The term “immiscible to non-dispersible in aqueous medium or bodily fluids” refers to a substance or mixture that does completely dissolves or uniformly distribute throughout an aqueous medium or body fluid.

The term “dissipation into body fluid upon placement within a body tissue” refers to a substance that when a quantity is emplaced within body tissue dissolves or disperses away from the original site of emplacement.

The term “diffusion into body fluid upon placement within a body tissue” refers to a substance that when a quantity is emplaced within body tissue dissolves or disperses in body fluid such that it is transported in the body fluid from the original site of emplacement.

The term “absorption into body fluid upon placement within a body tissue” refers to a substance that when a quantity is emplaced within body tissue is taken up by body fluid such that it is transported in the body fluid from the original site of emplacement.

The term “degradation in body fluid upon placement within a body tissue” refers to a substance that when a quantity is emplaced within body tissue is chemically broken down in body fluid such that its chemical nature is altered.

The term “non-aqueous” refers to an absence of water. For instance, a non-aqueous liquid is a liquid that is substantially free of water, e.g., includes less that about 1 wt. % water.

The term “biodegradable” refers to a substance that is acted on by agents within living tissue such as enzymes to alter the chemical nature of the substance. For example, a biodegradable polymer can be broken down by tissue enzymes into components of low molecular weight such as monomeric fragments.

The term “thermoplastic polymer” refers to a polymer that when subjected to elevated temperatures, exhibits a decrease in viscosity or resistance to deformation.

The term “hydrophobic” refers to a substance, molecule, or a domain of a substance of molecule that does not dissolve or is not wetted by water. The energetic interaction between a hydrophobic substance, molecule, or domain is unfavorable. Examples include solvents such as hydrocarbons and esters that when mixed with water undergo phase separation.

The term “block copolymer” refers to a polymer composed of two or more different chemical types of monomer units, wherein monomer units of one type are largely associated only with each other in particular domains, “blocks,” of the polymer and monomer units of another type are also largely associated only with each other in other particular domains or blocks of the polymer. The backbone or continuous molecular chain of the polymer contains domains of at least two blocks.

The term “substantially insoluble in the biocompatible solvent system” refers to a substance or mixture that does not dissolve to any significant degree in a solvent or mixture of solvents that are biocompatible, as defined above. In specific embodiments, less than about 5 wt. % of the substance or mixture (e.g., active pharmaceutical ingredient) completely dissolves in the biocompatible solvent system. In further specific embodiments, less than about 1 wt. % of the substance or mixture (e.g., active pharmaceutical ingredient) completely dissolves in the biocompatible solvent system. In further specific embodiments, less than about 0.1 wt. % of the substance or mixture (e.g., active pharmaceutical ingredient) completely dissolves in the biocompatible solvent system.

The term “substantially insoluble in water or bodily fluids” refers to a substance or mixture that does not dissolve to any significant degree in water or in body fluids such as blood, intercellular fluid, or the like. In specific embodiments, less than about 5 wt. % of the substance or mixture (e.g., active pharmaceutical ingredient) completely dissolves in water or in body fluids. In further specific embodiments, less than about 1 wt. % of the substance or mixture (e.g., active pharmaceutical ingredient) completely dissolves in water or in body fluids. In further specific embodiments, less than about 0.1 wt. % of the substance or mixture (e.g., active pharmaceutical ingredient) completely dissolves in water or in body fluids.

The term “monosaccharide units” refers to components of a polymer that is formed from carbohydrate, i.e., sugar, monomeric units. For example, glucose is a monosaccharide unit of the polymer starch.

The term “pendent groups” refers to chemical moieties or groups that are covalently bonded to a polymer backbone, but are not themselves part of the backbone or continuous molecular chain.

The term “unit” refers to a component of a polymer, which can be a repeating unit of the polymer or can occur irregularly within a polymer molecule.

The term “glucopyranose” refers to a molecule or unit of a polymer that is composed of glucose, i.e., a six-carbon sugar having a particular stereochemical arrangement of hydroxy groups as is well known in the art, wherein the molecule includes a six-membered pyran ring. Glucose can exist in two minor image forms, termed D-glucose and L-glucose. D-glucose is the widely distributed naturally occurring form found in starch, cellulose, and other natural polysaccharides. The chemical structure of a glycopyranose (here, the D-form, with the three adjacent hydroxyl groups projecting towards the viewer) is as shown in the following formula:

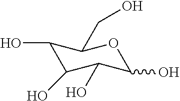

The hydroxyl group connected by the wavy line to the carbon atom at position 1 can be in either axial or equatorial configuration when glucose is in its free form; in aqueous solution the two forms are in equilibrium. These two forms are termed α and β anomeric forms. The α form has the structure:

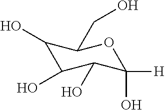

and the β form has the structure:

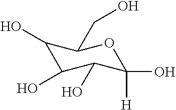

The term “glucopyranose units” refers to glucopyranose moieties contained within a polymeric structure. In specific embodiments, the glucopyranose units can be linked by α(1→4) glycosidic bonds. In other specific embodiments, the glucopyranose units can be linked by α(1→6) glycosidic bonds. In other specific embodiments, the glucopyranose units can be linked by a combination or mixture of α(1→4) glycosidic bonds and α(1→6) glycosidic bonds.

The term “glucopyranose monomeric units” refers to a glucopyranose unit that is incorporated into a polymeric polysaccharide formed of such units. In specific embodiments, the glucopyranose monomeric units can be linked by α(1→4) glycosidic bonds. In other specific embodiments, the glucopyranose monomeric units can be linked by α(1→6) glycosidic bonds. In other specific embodiments, the glucopyranose monomeric units can be linked by a combination or mixture of α(1→4) glycosidic bonds and α(1→6) glycosidic bonds.

The term “α(1→4) glycosidic bonds” refers to a covalent bond in a polysaccharide wherein a monosaccharide monomeric unit is bonded to a neighboring monomeric units via a hydroxy group on the carbon atom at position 1 coupled with a hydroxyl group at position 4 of a neighboring monosaccharide monomeric unit, wherein the hydroxyl group on the carbon atom at position 1 is in the axial configuration of a pyranose. The following formula shows a disaccharide formed of two D-glucose units bonded via an α(1→4) glycosidic bond:

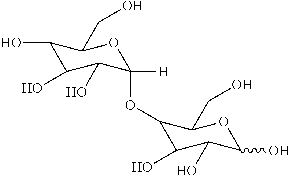

The term “α(1→6) glycosidic bonds” refers to a covalent bond in a polysaccharide wherein a monosaccharide monomeric unit is bonded to a neighboring monomeric units via a hydroxy group on the carbon atom at position 1 coupled with a hydroxyl group at position 6 of a neighboring monosaccharide monomeric unit, wherein the hydroxyl group on the carbon atom at position 1 is in the axial configuration of a pyranose. The following formula shows a disaccharide formed of two D-glucose units bonded via an α(1→6) glycosidic bond:

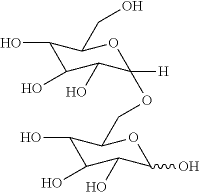

The term “D-glucopyranose” refers to a pyranose form of D-glucose. The formula is as shown below, with the three adjacent hydroxyl groups projecting in the direction of the viewer:

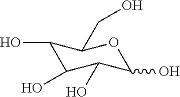

The term “non-macrocyclic poly-α(1→4) glucopyranose” refers to a polysaccharide composed of monosaccharide monomeric units joined by α(1→4) glucosidic bonds, wherein the polysaccharide does not form a macrocyclic ring.

The term “non-macrocyclic poly-α(1→6) glucopyranose” refers to a polysaccharide composed of monosaccharide monomeric units joined by α(1→6) glucosidic bonds, wherein the polysaccharide does not form a macrocyclic ring.

The term “homopolysaccharide” refers to a polysaccharide composed of only one type of monosaccharide repeating unit.

The term “heteropolysaccharide” refers to a polysaccharide composed of more than one type of monosaccharide repeating unit.

The term “natural polysaccharide (PS)” refers to a polysaccharide of natural origin. Examples include cellulose, starch, chitin, gum arabic, and the like.

The phrase “polysaccharide is non-macrocyclic” refers to a polysaccharide that does not include a macrocyclic ring (e.g., a ring that includes five or more monomeric saccharide units). Examples of non-macrocyclic polysaccharides include amylose and maltodextrins. An example of a macrocyclic polysaccharide is cyclodextrin, for example, α-, β-, or γ-cyclodextrin, which include six, seven, and eight monomeric saccharide units, respectively.

The phrase “polysaccharide is linear/branched” refers to a polysaccharide that is either linear, i.e., wherein all monosaccharide units are part of the backbone or continuous molecular chain of the polysaccharide, or branched, i.e., wherein some of the monosaccharide units are pendant from the backbone or the continuous molecular chain of the polysaccharide.

The term “substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, acyloxy, alkoxycarbonyl, amino, imino, alkylamino, acylamino, silyl ether, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyannato, sulfamoyl, sulfinamoyl, sulfino, sulfo, sulfoamino, thiosulfo, NRxRy and/or COORx, wherein each Rx and Ry are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

The term “partially unsaturated” refers to an organic group that contains one or more double bonds, and/or contains one or more triple bonds.

The term “degree of substitution” refers to a property of a polymer bearing a substituent or pendant group, wherein the degree of substitution is a numerical average of the number of such substituents or groups per monomeric unit of the polymer.

The term “metabolically cleavable covalent bond” refers to a chemical bond between two atoms that can be broken by the action of a naturally occurring agent in a living organism. For example, enzymes present in living tissues can act upon various different types of bonds to cleave them, often adding the elements of water across the bond (hydrolytic cleavage).

The term “metabolically cleavable carboxylic ester,” refers to ester groups that are metabolically cleavable as defined above.

The term “metabolically cleavable diester” refers to diesters wherein the ester bonds groups that are metabolically cleavable as defined above.

The term “metabolically cleavable carbonate” refers to carbonates, i.e., ROC(═O)OR′, groups that are metabolically cleavable as defined above.

The term “metabolically cleavable borate” refers to borate ester groups that are metabolically cleavable as defined above.

The term “metabolically cleavable silyl ether” refers to silicon esters groups that are metabolically cleavable as defined above.

The term “linear” in reference to a molecular structure refers to a chemical entity that is unbranched, i.e., wherein every monomeric unit is part of the backbone or continuous molecular chain.

The term “straight chain” refers to a linear molecular structure bearing no pendant side chains.

The term “branched” refers to a linear molecular structure bearing pendant side chains.

The term “amine terminated pendant group” refers to a pendant group as defined above that bears an amino group at its distal terminus.

The term “hydroxyl terminated pendant group” refers to a pendant group as defined above that bears a hydroxyl group at its distal terminus.

The term “subcutaneous” refers to underneath the skin of a living organism.

The term “subcutaneously” refers to administration of an agent to a tissue beneath the skin of an organism.

The term “parenteral” refers to a route of administration of an agent to a living organism other than oral or via the mouth.

The term “25 gauge needle” refers to a syringe needle for injection of solutions into living tissue of an external diameter defined as 25 gauge, as is well known in the art.

The term “suspension” or “dispersion” refers to a mixture of particles of a solid within a liquid, the particles being the dispersed phase, while the suspending medium is the continuous phase. The suspension can be a mixture of fine, nonsettling particles of a solid within a liquid. With a suspension, the particles are typically distributed through the liquid. With a suspension, the particles are typically not dissolved (i.e., are undissolved or unsolubilized) to a significant degree. The particles can be in microparticulate or nanoparticulate form. Additionally, the suspension can be a homogeneous suspension.

The term “homogeneous suspension” or “homogeneous dispersion” refers to a suspension or dispersion in which the particles are uniformly or essentially uniformly distributed through the liquid or solid, for example, at a macroscopic level.

The term “sucrose acetate isobutyrate (SAIB)” refers to a sucrose molecule, a disaccharide as is well known in the art, that bears at least one acetate ester group and at least one isobutyrate ester group. In specific embodiments, the injectable formulation will not include sucrose acetate isobutyrate (SAIB). In other specific embodiments, the injectable formulation will not include any appreciable or significant amount of sucrose acetate isobutyrate (SAIB). In such embodiments, the injectable formulation will include, e.g., less than about 1 wt. % sucrose acetate isobutyrate (SAIB).

The term “average particle size” refers to, in a population of particles, a numerical value representing the average diameter or major dimension of the population.

The term “little or no chemical interaction” refers to a situation wherein at least two molecular entities are in intimate contact but no reaction proceeds therebetween at any appreciable rate.

The term “liposome” refers to a structure, as is well known in the art, wherein a lipid bilayer or a plurality thereof encapsulates a volume of a liquid, such as an aqueous liquid, or a particulate solid, such as a drug particle.

The term “encapsulating” refers to enclosing a liquid or a solid within a coating of another material. In some embodiments, the coating can be substantially impermeable or completely impermeable for the practical purposes of the context in which the term is used.

The term “mammal” refers to an organism of the order Mammalia, including human beings, primates, hair-bearing non-primates such as dogs, cats, horses, cattle, marsupials, monotremes, and the like.

The term “essentially homogeneous implant” refers to a depot of a substance within living tissue of an organism wherein the implant is substantially uniform throughout.

The term “locally delivered” refers to a mode of delivery of a pharmaceutical substance from an implanted structure or depot to tissues predominantly in the vicinity of the implant within the organism. The pharmaceutical substance is delivered to a localized site in the subject but is not detectable at a biologically-significant level in the blood plasma of the subject.

The term “systemically delivered” refers to a mode of delivery of a pharmaceutical substance from an implanted structure or depot to tissues throughout the organism. The pharmaceutical substance is detectable at a biologically-significant level in the blood plasma of the subject.

The term “needle” refers to a syringe needle, as is well known in the art.

The term “zero-order release profile” refers to a rate of release of a bioactive substance from an implant wherein substantially the same amount per unit time of the substance is released for a period of time. A zero-order release profile is generally desirable as it provides for a substantially constant level of the bioactive substance in the bloodstream or tissue of the organism bearing the implant.

The term “burst” refers to a rate of release over time of a bioactive substance from an implant wherein the rate is not uniform, but is substantially greater during one segment of the period of time, typically immediately following emplacement of an implant bearing the bioactive substance in tissue.

The term “uniformly dispersed throughout the solid biodegradable implant” refers to an API contained within an implant, wherein the API is uniformly suspended throughout the implant.

### Biocompatible Solvent System

The biocompatible solvent system can include one or more (e.g., 1, 2, 3 or 4) specific solvents, for use in solubilizing or dissolving the biodegradable polymer. Any suitable solvent system can be employed, provided the biodegradable polymer is substantially soluble in the solvent system and provided the active pharmaceutical ingredient (API) is substantially insoluble in the solvent system.

Typically, the solvent system will include one, two, three, four or more liquids in which the biodegradable polymer is substantially soluble in the solvent system, but in which the active pharmaceutical ingredient (API) is substantially insoluble in the solvent system. Additionally, the solvent system will typically be liquid at ambient and physiological temperature.

The solvent system can have a solubility range of miscible to dispersible in aqueous medium or bodily fluids. Alternatively, the solvent system can have a solubility range of immiscible to non-dispersible in aqueous medium or bodily fluids. More specifically, the solvent system can be water immiscible.

The solvent system can include at least one organic solvent that is miscible to dispersible in aqueous medium or body fluid. Alternatively, the solvent system can include at least one organic solvent that is immiscible to insoluble in aqueous medium or body fluid. Alternatively, the solvent system can include a combination of at least one organic solvent that is miscible to dispersible in aqueous medium or body fluid, and at least one organic solvent that is immiscible to insoluble in aqueous medium or body fluid. Alternatively, the solvent system can include a combination of at least one organic solvent that is miscible to dispersible in aqueous medium or body fluid, and at least one organic solvent that is immiscible to insoluble in aqueous medium or body fluid, wherein the polymer has greater solubility in the miscible to dispersible solvent, as compared to the immiscible to insoluble solvent.

The solvent system can be capable of dissipation, diffusion, absorption, degradation, or a combination thereof, into body fluid upon placement within a body tissue. Additionally, the solvent system can include at least one biodegradable organic solvent.

The solvent system can be non-aqueous. Specifically, the solvent system can include one or more organic compounds. Each of the one or more organic compounds can be a liquid at ambient and physiological temperature. Additionally, the solvent system can include at least one aprotic solvent.

In one embodiment, the solvent system includes at least one aliphatic ester.

Suitable classes of compounds for use in the solvent system include, for example, alkyl esters, aryl esters, glycerol, diesters, triesters, benzyl alcohols, propylene glycols, or a combination or mixture thereof.

Suitable specific compounds for use in the solvent system include, for example, ethyl heptanoate, ethyl octanoate, glycofural, benzyl benzoate, glycerol tributyrate, dimethyl isosorbide, glycerol triacetate (triacetin), glycerol tributyrate, and a combination or mixture thereof.

In a specific embodiment, the solvent system does not include any significant or appreciable amount of any one or more of the following compounds: dimethyl sulfoxide (DMSO), N-methyl-2-pyrrolidone (NMP), methanol, ethanol, isopropyl alcohol, dimethylformamide (DMF) and dimethylacetamide (DMAC).

Additional compounds for use in the solvent system are disclosed and commercially available from, e.g., Aldrich Handbook of Fine Chemicals and Laboratory Equipment, Milwaukee, Wis. (2009).

The suitable biocompatible solvent system should also be able to diffuse into body fluid, so that the composition can effectively coagulate or solidify in vivo.

The solvent system can be present in any suitable and effective amount, provided the biodegradable polymer is substantially soluble in the solvent system and provided the active pharmaceutical ingredient (API) is substantially insoluble in the solvent system. The type and amount of solvent system present in the composition will typically depend upon the desired properties of the controlled release implant. For example, the type and amount of solvent system can influence the length of time in which the active pharmaceutical ingredient (API) is released from the controlled release implant.

In one embodiment, the solvent system is present in about 10 wt. % to about 40 wt. % of the formulation. In another embodiment, the solvent system is present in about 40 wt. % to about 90 wt. % of the formulation.

### Biodegradable Polymer

The biodegradable polymer can include one or more (e.g., 1, 2, 3 or 4) specific biodegradable polymers, for use in forming an implant in vivo. Suitable polymers will be biodegradable and will be substantially soluble in the biocompatible solvent system. Specifically, the biodegradable polymer can have a solubility of at least about 50 g/L in the biocompatible solvent system, at 25° C. and 1 atm. In one embodiment, the biodegradable polymer will not include a polymer that is substantially insoluble in the biocompatible solvent system. In another embodiment, the biodegradable polymer will not include a biodegradable polymer that is substantially insoluble in water or bodily fluids.

Suitable specific classes of polymers include, e.g., polylactides, polyglycolides, polycaprolactones, polyanhydrides, polyamines, polyurethanes, polyesteramides, polyorthoesters, polydioxanones, polyacetals, polyketals, polycarbonates, polyorthocarbonates, polyphosphazenes, succinates, poly(malic acid), poly(amino acids), polyvinylpyrrolidone, polyethylene glycol, polyhydroxycellulose, polysaccharides, chitin, chitosan, and copolymers, block copolymers, multi-block co-polymers, multi-block co-polymers with polyethylene glycol (PEG), polyols, terpolymers and mixtures thereof.

In one embodiment, the biodegradable polymer is a thermoplastic polymer.

In one embodiment, the biodegradable polymer has a viscosity of at least about 100 cP at 37° C. In other embodiments, the biodegradable polymer has a viscosity of about 1,000 cP to about 30,000 cp at 37° C., about 5,000 cP to about 25,000 cp at 37° C., or about 10,000 cP to about 20,000 cp at 37° C.

In one embodiment, the biodegradable polymer is hydrophobic.

In one embodiment, the biodegradable polymer includes a block copolymer. In another embodiment, the biodegradable polymer is a polyethylene glycol (PEG) containing tri-block co-polymer.

In one embodiment the polymer contains functional side groups.

The biodegradable polymer can be present in any suitable and effective amount, provided the biodegradable polymer is substantially soluble in the solvent system, and in combination with the solvent system will form an implant in vivo. In one embodiment, the biodegradable polymer is present in about 10 wt. % to about 40 wt. % of the formulation. In another embodiment, the biodegradable polymer is present in about 40 wt. % to about 90 wt. % of the formulation.

In one embodiment, the biodegradable polymer can include a poly(ether ester) multi-block copolymer. In another embodiment, the biodegradable polymer can include a biodegradable polysaccharides polymer. In another embodiment, the biodegradable polymer can include a polyglycerol fatty acid ester. In another embodiment, the biodegradable polymer can include a PEG-PBT polymer. In another embodiment, the biodegradable polymer can include a polyester amide. In another embodiment, the biodegradable polymer can include a poly(ester-amide) polymer (PEA).

Poly(ether ester) Polymers

One suitable class of biodegradable polymers useful in the present invention includes the poly(ether ester) poly(ether ester) multi-block copolymers. These multi-block copolymers are composed of various pre-polymer building blocks of different combinations of DL-lactide, glycolide, ε-caprolactone and polyethylene glycol. By varying the molecular composition, molecular weight (Mw 1200-6000) and ratio of the pre-polymer blocks, different functionalities can be introduced into the final polymer, which enables the creation of polymers with various physio-chemical properties. Both hydrophobic as well as hydrophilic/swellable polymers and slowly degrading as well as rapidly degrading polymers can be designed.

The poly(ether ester) multi-block copolymers can include a polymer as shown below (formula III):

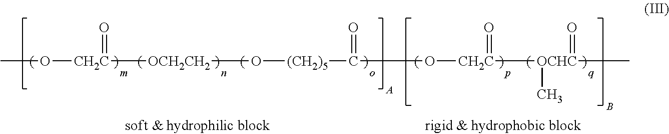

wherein,

m and p are each independently glycolide;

n is polyethylene glycol, Mw 300-1000;

o is ε-caprolactone; and

q is DL-lactide.

Under physiological conditions, poly(ether ester) multi-block copolymers can degrade completely via hydrolysis into non-toxic degradation products which are metabolized and/or excreted through the urinary pathway. Consequently, there can be no accumulation of biomaterials, thereby minimizing the chance of long-term foreign body reactions.

Additional features and descriptions of the poly(ether ester) multi-block copolymers are provided, for example, in Published PCT Patent Application No. WO 2005/068533 and references cited therein. An overview is provided below.

The multi-block copolymers can specifically include two hydrolysable segments having a different composition, linked by a multifunctional, specifically an aliphatic chain-extender, and which are specifically essentially completely amorphous under physiological conditions (moist environment, body temperature, which is approximately 37° C. for humans).

The resulting multi-block copolymers can specifically have a structure according to any of the formulae (1)-(3):

[—R₁-Q1-R4-Q2-]ₓ-[R2-Q3-R4-Q4-]_{y}-[R3-Q5-R₄-Q6-]_{z}- (1)

[—R₁—R₂—R₁-Q1-R₄-Q2-]ₓ-[R₃-Q2-R₄-Q1]_{z}- (2)

[—R₂—R₁—R₂-Q1-R₄-Q2-]ₓ[R₃-Q2-R₄-Q1]z- (3)

wherein

R₁ and R₂ can be amorphous polyester, amorphous poly ether ester or amorphous polycarbonate; or an amorphous pre-polymer that is obtained from combined ester, ether and/or carbonate groups. R₁ and R₂ can contain polyether groups, which can result from the use of these compounds as a polymerization initiator, the polyether being amorphous or crystalline at room temperature. However, the polyether thus introduced will become amorphous at physiological conditions. R₁ and R₂ are derived from amorphous pre-polymers or blocks A and B, respectively, and R₁ and R₂ are not the same. R₁ and R₂ can contain a polyether group at the same time. In a specific embodiment, only one of them will contain a polyether group;

z is zero or a positive integer;

R₃ is a polyether, such as poly(ethylene glycol), and may be present (z≠0) or not (z=0). R₃ will become amorphous under physiological conditions;

R₄ is an aliphatic C₂-C₈ alkylene group, optionally substituted by a C₁-C₁₀ alkylene, the aliphatic group being linear or cyclic, wherein R₄ can specifically be a butylene, —(CH₂)₄— group, and the C₁-C₁₀ alkylene side group can contain protected S, N, P or O moieties;

x and y are both positive integers, which can both specifically be at least 1, whereas the sum of x and y (x+y) can specifically be at most 1000, more specifically at most 500, or at most 100. Q1-Q6 are linking units obtained by the reaction of the pre-polymers with the multifunctional chain-extender. Q1-Q6 are independently amine, urethane, amide, carbonate, ester or anhydride. The event that all linking groups Q are different being rare and not preferred.

Typically, one type of chain-extender can be used with three pre-polymers having the same end-groups, resulting in a copolymer of formula (1) with six similar linking groups. In case pre-polymers R₁ and R₂ are differently terminated, two types of groups Q will be present: e.g. Q1 and Q2 will be the same between two linked pre-polymer segments R₁, but Q1 and Q2 are different when R₁ and R₂ are linked. Obviously, when Q1 and Q2 are the same, it means that they are the same type of group but as minor images of each other.

In copolymers of formula (2) and (3) the groups Q1 and Q2 are the same when two pre-polymers are present that are both terminated with the same end-group (which is usually hydroxyl) but are different when the pre-polymers are differently terminated (e.g. PEG which is diol terminated and a di-acid terminated ‘tri-block’ pre-polymer). In case of the tri-block pre-polymers (R₁R₂R₁ and R₂R₁R₂), the outer segments should be essentially free of PEG, because the coupling reaction by ring opening can otherwise not be carried out successfully. Only the inner block can be initiated by a PEG molecule.

The examples of formula (1), (2) and (3) show the result of the reaction with a di-functional chain-extender and di-functional pre-polymers.

With reference to formula (1) the polyesters can also be represented as multi-block or segmented copolymers having a structure (ab)n with alternating a and b segments or a structure (ab)r with a random distribution of segments a and b, wherein ‘a’ corresponds to the segment R₁ derived from pre-polymer (A) and ‘b’ corresponds to the segment R₂ derived from pre-polymer (B) (for z=0). In (ab)r, the a/b ratio (corresponding to x/y in formula (1)) may be unity or away from unity. The pre-polymers can be mixed in any desired amount and can be coupled by a multifunctional chain extender, viz. a compound having at least two functional groups by which it can be used to chemically link the pre-polymers. Specifically, this is a di-functional chain-extender. In case z≠0, then the presentation of a random distribution of all the segments can be given by (abc)r were three different pre-polymers (one being e.g. a polyethylene glycol) are randomly distributed in all possible ratio's. The alternating distribution is given by (abc)n. In this particular case, alternating means that two equally terminated pre-polymers (either a and c or b and c) are alternated with a differently terminated pre-polymer b or a, respectively, in an equivalent amount (a+c=b or b+c=a). Those according to formula (2) or (3) have a structure (aba)n and (bab)n wherein the aba and bab ‘triblock’ pre-polymers are chain-extended with a di-functional molecule.

The method to obtain a copolymer with a random distribution of a and b (and optionally c) is far more advantageous than when the segments are alternating in the copolymer such as in (ab)n with the ratio of pre-polymers a and b being 1. The composition of the copolymer can then only be determined by adjusting the pre-polymer lengths. In general, the a and b segment lengths in (ab)n alternating copolymers are smaller than blocks in block-copolymers with structures ABA or AB.

The pre-polymers of which the a and b (and optionally c) segments are formed in (ab)r, (abc)r, (ab)n and (abc)n are linked by the di-functional chain-extender. This chain-extender can specifically be a diisocyanate chain-extender, but can also be a diacid or diol compound. In case all pre-polymers contain hydroxyl end-groups, the linking units will be urethane groups. In case (one of) the pre-polymers are carboxylic acid terminated, the linking units are amide groups. Multi-block copolymers with structure (ab)r and (abc)r can also be prepared by reaction of di-carboxylic acid terminated pre-polymers with a diol chain extender or vice versa (diol terminated pre-polymer with diacid chain-extender) using a coupling agent such as DCC (dicyclohexyl carbodiimide) forming ester linkages. In (aba)n and (bab)n the aba and bab pre-polymers are also specifically linked by an aliphatic di-functional chain-extender, more specifically, a diisocyanate chain-extender.

The term “randomly segmented” copolymers refers to copolymers that have a random distribution (i.e. not alternating) of the segments a and b: (ab)r or a, b and c: (abc)r.

### PEG-PBT Polymers

One suitable class of biodegradable polymers useful in the present invention include the poly(ether ester) multiblock copolymers based on poly(ethylene glycol) (PEG) and poly(butylene terephthalate) (PBT), that can be described by the following general formula IV:

[—(OCH₂CH₂)ₙ—O—C(O)—C₆H₄—C(O)—]ₓ[—O—(CH₂)₄—O—C(O)—C₆H₄—C(O)—]_{y}, (IV)

wherein,

—C₆H₄— designates the divalent aromatic ring residue from each esterified molecule of terephthalic acid,

n represents the number of ethylene oxide units in each hydrophilic PEG block,

x represents the number of hydrophilic blocks in the copolymer, and

y represents the number of hydrophobic blocks in the copolymer.

In specific embodiments, n can be selected such that the molecular weight of the PEG block is between about 300 and about 4000. In specific embodiments, x and y can each be independently selected so that the multiblock copolymer contains from about 55% up to about 80% PEG by weight.

The block copolymer can be engineered to provide a wide array of physical characteristics (e.g., hydrophilicity, adherence, strength, malleability, degradability, durability, flexibility) and bioactive agent release characteristics (e.g., through controlled polymer degradation and swelling) by varying the values of n, x and y in the copolymer structure.

### Polyester Amides

One suitable class of biodegradable polymers useful in the present invention includes the polyesteramide polymers having a subunit of the formula (V):

—[—O—(CH₂)ₓ—O—C(O)—CHR—NH—C(O)—(CH₂)_{y}—C(O)—NH—CHR—C(O)—]— (V)

wherein,

x is C₂-C₁₂,

y is C₂-C₁₂, and

R is —CH(CH₃)₂, —CH₂CH(CH₃)₂, —CH(CH₃)CH₂CH₃, —CH₂(CH₂)₂CH₃, —CH₂C₆H₅, —CH₂(CH₂)₂SCH₃ or part of an amino acid.

In specific embodiments, the C₂-C₁₂ can be (C₂-C₁₂) alkyl. In other specific embodiments, the C₂-C₁₂ can be (C₂-C₁₂) alkyl, optionally substituted.

Such polymers are described, for example, in U.S. Pat. No. 6,703,040. Polymers of this nature can be described with a nomenclature of x-aa-y, wherein “x” represents an alkyl diol with x carbon atoms, “aa” represents an amino acid such as leucine or phenylalanine, and y represents an alkyldicarboxylic acid with y carbon atoms, and wherein the polymer is a polymerization of the diol, the dicarboxylic acid, and the amino acid. An exemplary polymer of this type is 4-Leu-4.

### Poly(ester-amide) Polymer (PEA)

One suitable class of biodegradable polymers useful in the present invention includes the poly(ester-amide) polymers. Such polymers can be prepared by polymerization of a diol, a dicarboxylic acid and an alpha-amino acid through ester and amide links in the form (DACA)ₙ. An example of a (DACA)ₙ polymer is shown below in formula VI. Suitable amino acids include any natural or synthetic alpha-amino acid, specifically neutral amino acids.

Diols can be any aliphatic diol, including alkylene diols like HO—(CH₂)ₖ—OH (i.e. non-branched), branched diols (e.g., propylene glycol), cyclic diols (e.g. dianhydrohexitols and cyclohexanediol), or oligomeric diols based on ethylene glycol (e.g., diethylene glycol, triethylene glycol, tetraethylene glycol, or poly(ethylene glycol)s). Aromatic diols (e.g. bis-phenols) are less useful for these purposes since they are more toxic, and polymers based on them have rigid chains that are less likely to biodegrade.

Dicarboxylic acids can be any aliphatic dicarboxylic acid, such as α-omega-dicarboxylic acids (i.e., non-branched), branched dicarboxylic acids, cyclic dicarboxylic acids (e.g. cyclohexanedicarboxylic acid). Aromatic diacids (like phthalic acids, etc.) are less useful for these purposes since they are more toxic, and polymers based on them have rigid chain structure, exhibit poorer film-forming properties and have much lower tendency to biodegrade.

Specific PEA polymers have the formula VI:

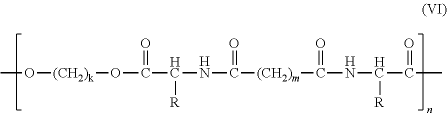

wherein,

k is 2-12 (e.g., 2, 3, 4, or 6);

m is 2-12 (e.g., 4 or 8); and

R is —CH(CH₃)₂, —CH₂CH(CH₃)₂, —CH(CH₃)CH₂CH₃, —CH₂(CH₂)₂CH₃, —CH₂(C₆H₅), or —CH₂(CH₂)SCH₃.

In specific embodiments, A is L-phenylalanine (Phe-PEA) and A is L-leucine (Leu-PEA). In specific embodiments, the ratio of Phe-PEA to Leu-PEA is from 10:1 to 1:1. In other specific embodiments, the ratio of Phe-PEA to Leu-PEA is from 5:1 to 2.5:1.

Additional features and descriptions of the poly(ester-amide) polymers (PEA) are provided, for example, in US Re40,359, which is a reissue of U.S. Pat. No. 6,703,040.

### Biodegradable Polysaccharides Polymers

One suitable class of biodegradable polymers useful in the present invention includes the hydrophobic derivatives of natural biodegradable polysaccharides. Hydrophobic derivatives of natural biodegradable polysaccharide refer to a natural biodegradable polysaccharide having one or more hydrophobic pendent groups attached to the polysaccharide. In many cases the hydrophobic derivative includes a plurality of groups that include hydrocarbon segments attached to the polysaccharide. When a plurality of groups including hydrocarbon segments are attached, they are collectively referred to as the “hydrophobic portion” of the hydrophobic derivative. The hydrophobic derivatives therefore include a hydrophobic portion and a polysaccharide portion.

The polysaccharide portion includes a natural biodegradable polysaccharide, which refers to a non-synthetic polysaccharide that is capable of being enzymatically degraded. Natural biodegradable polysaccharides include polysaccharide and/or polysaccharide derivatives that are obtained from natural sources, such as plants or animals. Natural biodegradable polysaccharides include any polysaccharide that has been processed or modified from a natural biodegradable polysaccharide (for example, maltodextrin is a natural biodegradable polysaccharide that is processed from starch). Exemplary natural biodegradable polysaccharides include maltodextrin, amylose, cyclodextrin, polyalditol, hyaluronic acid, dextran, heparin, chondroitin sulfate, dermatan sulfate, heparan sulfate, keratan sulfate, dextran, dextran sulfate, pentosan polysulfate, and chitosan. Specific polysaccharides are low molecular weight polymers that have little or no branching, such as those that are derived from and/or found in starch preparations, for example, maltodextrin, amylose, and cyclodextrin. Therefore, the natural biodegradable polysaccharide can be a substantially non-branched or completely non-branched poly(glucopyranose) polymer.

“Amylose” or “amylose polymer” refers to a linear polymer having repeating glucopyranose units that are joined by α-1,4 linkages. Some amylose polymers can have a very small amount of branching via α-1,6 linkages (about less than 0.5% of the linkages) but still demonstrate the same physical properties as linear (unbranched) amylose polymers do. Generally amylose polymers derived from plant sources have molecular weights of about 1×10⁶ Da or less. Amylopectin, comparatively, is a branched polymer having repeating glucopyranose units that are joined by α-1,4 linkages to form linear portions and the linear portions are linked together via α-1,6 linkages. The branch point linkages are generally greater than 1% of the total linkages and typically 4%-5% of the total linkages. Generally amylopectin derived from plant sources have molecular weights of 1×10⁷ Da or greater.

For example, in some aspects, starch preparations having a high amylose content, purified amylose, synthetically prepared amylose, or enriched amylose preparations can be used in the preparation of a hydrophobic derivative of amylose. In starch sources, amylose is typically present along with amylopectin, which is a branched polysaccharide. If a mixture of amylose and a higher molecular weight precursor is used (such as amylopectin), amylose can be present in the composition in an amount greater than the higher molecular weight precursor. For example, in some aspects, starch preparations having high amylose content, purified amylose, synthetically prepared amylose, or enriched amylose preparations can be used in the preparation of a hydrophobic derivative of amylose polymer. In some embodiments the composition includes a mixture of polysaccharides including amylose wherein the amylose content in the mixture of polysaccharides is 50% or greater, 60% or greater, 70% or greater, 80% or greater, or 85% or greater by weight. In other embodiments the composition includes a mixture of polysaccharides including amylose and amylopectin and wherein the amylopectin content in the mixture of polysaccharides is 30% or less, or 15% or less.

The amount of amylopectin present in a starch may also be reduced by treating the starch with amylopectinase, which cleaves a-1,6 linkages resulting in the debranching of amylopectin into amylose.

Steps may be performed before, during, and/or after the process of derivatizing the amylose polymer with a pendent group comprising a hydrocarbon segment to enrich the amount of amylose, or purify the amylose.

Amylose of particular molecular weights can be obtained commercially or can be prepared. For example, synthetic amyloses with average molecular masses of 70 kDa, 110 kDa, and 320 kDa, can be obtained from Nakano Vinegar Co., Ltd. (Aichi, Japan). The decision of using amylose of a particular size range may depend on factors such as the physical characteristics of the composition (e.g., viscosity), the desired rate of degradation of the implant, and the nature and amount of the active pharmaceutical ingredient (API).

Purified or enriched amylose preparations can be obtained commercially or can be prepared using standard biochemical techniques such as chromatography. In some aspects, high-amylose cornstarch can be used to prepare the hydrophobic derivative.

Maltodextrin is typically generated by hydrolyzing a starch slurry with heat-stable α-amylase at temperatures at 85-90° C. until the desired degree of hydrolysis is reached and then inactivating the α-amylase by a second heat treatment. The maltodextrin can be purified by filtration and then spray dried to a final product. Maltodextrins are typically characterized by their dextrose equivalent (DE) value, which is related to the degree of hydrolysis defined as: DE=MW dextrose/number-averaged MW starch hydrolysate×100. Generally, maltodextrins are considered to have molecular weights that are less than amylose molecules.

A starch preparation that has been totally hydrolyzed to dextrose (glucose) has a DE of 100, whereas starch has a DE of about zero. A DE of greater than 0 but less than 100 characterizes the mean-average molecular weight of a starch hydrolysate, and maltodextrins are considered to have a DE of less than 20. Maltodextrins of various molecular weights, for example, in the range of about 500 Da to 5000 Da are commercially available (for example, from CarboMer, San Diego, Calif.).

Another contemplated class of natural biodegradable polysaccharides is natural biodegradable non-reducing polysaccharides. A non-reducing polysaccharide can provide an inert matrix thereby improving the stability of active pharmaceutical ingredients (APIs), such as proteins and enzymes. A non-reducing polysaccharide refers to a polymer of non-reducing disaccharides (two monosaccharides linked through their anomeric centers) such as trehalose (α-D-glucopyranosyl α-D-glucopyranoside) and sucrose (β-D-fructofuranosyl α-D-glucopyranoside). An exemplary non-reducing polysaccharide includes polyalditol which is available from GPC (Muscatine, Iowa). In another aspect, the polysaccharide is a glucopyranosyl polymer, such as a polymer that includes repeating (1→3)O-β-D-glucopyranosyl units.

Dextran is an α-D-1,6-glucose-linked glucan with side-chains 1-3 linked to the backbone units of the dextran biopolymer. Dextran includes hydroxyl groups at the 2, 3, and 4 positions on the glucopyranose monomeric units. Dextran can be obtained from fermentation of sucrose-containing media by Leuconostoc mesenteroides B512F.

Dextran can be obtained in low molecular weight preparations. Enzymes (dextranases) from molds such as Penicillium and Verticillium have been shown to degrade dextran. Similarly many bacteria produce extracellular dextranases that split dextran into low molecular weight sugars.

Chondroitin sulfate includes the repeating disaccharide units of D-galactosamine and D-glucuronic acid, and typically contains between 15 to 150 of these repeating units. Chondroitinase AC cleaves chondroitin sulfates A and C, and chondroitin.

Hyaluronic acid (HA) is a naturally derived linear polymer that includes alternating β-1,4-glucuronic acid and β-1,3-N-acetyl-D-glucosamine units. HA is the principal glycosaminoglycan in connective tissue fluids. HA can be fragmented in the presence of hyaluronidase.

In many aspects the polysaccharide portion and the hydrophobic portion include the predominant portion of the hydrophobic derivative of the natural biodegradable polysaccharide. Based on a weight percentage, the polysaccharide portion can be about 25% wt of the hydrophobic derivative or greater, in the range of about 25% to about 75%, in the range of about 30% to about 70%, in the range of about 35% to about 65%, in the range of about 40% to about 60%, or in the range of about 45% to about 55%. Likewise, based on a weight percentage of the overall hydrophobic derivative, the hydrophobic portion can be about 25% wt of the hydrophobic derivative or greater, in the range of about 25% to about 75%, in the range of about 30% to about 70%, in the range of about 35% to about 65%, in the range of about 40% to about 60%, or in the range of about 45% to about 55%. In exemplary aspects, the hydrophobic derivative has approximately 50% of its weight attributable to the polysaccharide portion, and approximately 50% of its weight attributable to its hydrophobic portion.

The hydrophobic derivative has the properties of being insoluble in water. The term for insolubility is a standard term used in the art, and meaning 1 part solute per 10,000 parts or greater solvent. (see, for example, Remington: The Science and Practice of Pharmacy, 20th ed. (2000), Lippincott Williams & Wilkins, Baltimore Md.).

A hydrophobic derivative can be prepared by associating one or more hydrophobic compound(s) with a natural biodegradable polysaccharide polymer. Methods for preparing hydrophobic derivatives of natural biodegradable polysaccharides are described herein.

The hydrophobic derivatives of the natural biodegradable polysaccharides specifically have an average molecular weight of up to about 1,000,000 Da, up to about 300,000 Da or up to about 100,000 Da. Use of these molecular weight derivatives can provide implants with desirable physical and drug-releasing properties. In some aspects the hydrophobic derivatives have a molecular weight of about 250,000 Da or less, about 100,000 Da or less, about 50,000 Da or less, or 25,000 Da or less. Particularly specific size ranges for the natural biodegradable polysaccharides are in the range of about 2,000 Da to about 20,000 Da, or about 4,000 Da to about 10,000 Da.

The molecular weight of the polymer is more precisely defined as “weight average molecular weight” or M_{w}. M_{w} is an absolute method of measuring molecular weight and is particularly useful for measuring the molecular weight of a polymer (preparation). Polymer preparations typically include polymers that individually have minor variations in molecular weight. Polymers are molecules that have a relatively high molecular weight and such minor variations within the polymer preparation do not affect the overall properties of the polymer preparation. The M_{w} can be measured using common techniques, such as light scattering or ultracentrifilgation. Discussion of M_{w} and other terms used to define the molecular weight of polymer preparations can be found in, for example, Allcock, H. R. and Lampe, F. W. (1990) Contemporary Polymer Chemistry; pg 271.

The addition of hydrophobic portion will generally cause an increase in molecular weight of the polysaccharide from its underivitized, starting molecular weight. The amount increase in molecular weight can depend on one or more factors, including the type of polysaccharide derivatized, the level of derivation, and, for example, the type or types of groups attached to the polysaccharide to provide the hydrophobic portion.

In some aspects, the addition of hydrophobic portion causes an increase in molecular weight of the polysaccharide of about 20% or greater, about 50% or greater, about 75% or greater, about 100% or greater, or about 125%, the increase in relation to the underivitized form of the polysaccharide.

As an example, a maltodextrin having a starting weight of about 3000 Da is derivitized to provide pendent hexanoate groups that are coupled to the polysaccharide via ester linkages to provide a degree of substitution (DS) of about 2.5. This provides a hydrophobic polysaccharide having a theoretical molecular weight of about 8400 Da.

In forming the hydrophobic derivative of the natural biodegradable polysaccharide and as an example, a compound having a hydrocarbon segment can be covalently coupled to one or more portions of the polysaccharide. For example, the compound can be coupled to monomeric units along the length of the polysaccharide. This provides a polysaccharide derivative with one or more pendent groups. Each chemical group includes a hydrocarbon segment. The hydrocarbon segment can constitute all of the pendent chemical group, or the hydrocarbon segment can constitute a portion of the pendent chemical group. For example, a portion of the hydrophobic polysaccharide can have the following structural formula (I):

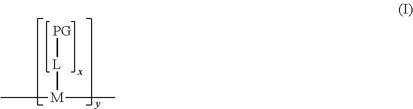

wherein each M is independently a monosaccharide unit, each L is independently a suitable linking group, or is a direct bond, each PG is independently a pendent group, each x is independently 0 to about 3, such that when x is 0, the bond between L and M is absent, and y is 3 or more.

Additionally, the polysaccharide that includes the unit of formula (I) above can be a compound of formula (II):

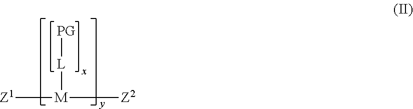

wherein each M is independently a monosaccharide unit, each L is independently a suitable linking group, or is a direct bond, each PG is independently a pendent group, each x is independently 0 to about 3, such that when x is 0, the bond between L and M is absent, y is about 3 to about 5,000, and Z¹ and Z² are each independently hydrogen, OR¹, OC(═O)R¹, CH₂OR¹, SiR¹ or CH₂OC(═O)R¹. Each R¹ is independently hydrogen, alkyl, cycloalkyl, cycloalkyl alkyl, aryl, aryl alkyl, heterocyclyl or heteroaryl, each alkyl, cycloalkyl, aryl, heterocycle and heteroaryl is optionally substituted, and each alkyl, cycloalkyl and heterocycle is optionally partially unsaturated.

For the compounds of formula (I) and (II), the monosaccharide unit (M) can include D-glucopyranose (e.g., α-D-glucopyranose). Additionally, the monosaccharide unit (M) can include non-macrocyclic poly-α(1→4) glucopyranose, non-macrocyclic poly-α(1→6) glucopyranose, or a mixture or combination of both non-macrocyclic poly-α(1→4) glucopyranose and non-macrocyclic poly-α(1→6) glucopyranose. For example, the monosaccharide unit (M) can include glucopyranose units, wherein at least about 90% are linked by α(1→4) glycosidic bonds. Alternatively, the monosaccharide unit (M) can include glucopyranose units, wherein at least about 90% are linked by α(1→6) glycosidic bonds. Additionally, each of the monosaccharides in the polysaccharide can be the same type (homopolysaccharide), or the monosaccharides in the polysaccharide can differ (heteropolysaccharide).

The polysaccharide can include up to about 5,000 monosaccharide units (i.e., y in the formula (I) or (II) is up to 5,000). Specifically, the monosaccharide units can be glucopyranose units (e.g., α-D-glucopyranose units). Additionally, y in the formula (I) or (II) can specifically be about 3-5,000 or about 3-4,000 or about 100 to 4,000.

In specific embodiments, the polysaccharide is non-macrocyclic. In other specific embodiments, the polysaccharide is linear. In other specific embodiments, the polysaccharide is branched. In yet further specific embodiments, the polysaccharide is a natural polysaccharide (PS).

The polysaccharide will have a suitable glass transition temperature (Tg). In one embodiment, the polysaccharide will have a glass transition temperature (Tg) of at least about 35° C. (e.g., about 40° C. to about 150° C.). In another embodiment, the polysaccharide will have a glass transition temperature (Tg) of −30° C. to about 0° C.

A “pendant group” refers to a group of covalently bonded carbon atoms having the formula (CHₙ)ₘ, wherein m is 2 or greater, and n is independently 2 or 1. A hydrocarbon segment can include saturated hydrocarbon groups or unsaturated hydrocarbon groups, and examples thereof include alkyl, alkenyl, alkynyl, cyclic alkyl, cyclic alkenyl, aromatic hydrocarbon and aralkyl groups. Specifically, the pendant group includes linear, straight chain or branched C₁-C₂₀ alkyl group; an amine terminated hydrocarbon or a hydroxyl terminated hydrocarbon. In another embodiment, the pendant group includes polyesters such as polylactides, polyglycolides, poly (lactide-co-glycolide) co-polymers, polycaprolactone, terpolymers of poly (lactide-co-glycolide-co-caprolatone), or combinations thereof.

The monomeric units of the hydrophobic polysaccharides described herein typically include monomeric units having ring structures with one or more reactive groups. These reactive groups are exemplified by hydroxyl groups, such as the ones that are present on glucopyranose-based monomeric units, e.g., of amylose and maltodextrin. These hydroxyl groups can be reacted with a compound that includes a hydrocarbon segment and a group that is reactive with the hydroxyl group (a hydroxyl-reactive group).

Examples of hydroxyl reactive groups include acetal, carboxyl, anhydride, acid halide, and the like. These groups can be used to form a hydrolytically cleavable covalent bond between the hydrocarbon segment and the polysaccharide backbone. For example, the method can provide a pendent group having a hydrocarbon segment, the pendent group linked to the polysaccharide backbone with a cleavable ester bond. In these aspects, the synthesized hydrophobic derivative of the natural biodegradable polysaccharide can include chemical linkages that are both enzymatically cleavable (the polymer backbone) and non-enzymatically hydrolytically cleavable (the linkage between the pendent group and the polymer backbone).

Other cleavable chemical linkages (e.g., metabolically cleavable covalent bonds) that can be used to bond the pendent groups to the polysaccharide include carboxylic ester, carbonate, borate, silyl ether, peroxyester groups, disulfide groups, and hydrazone groups.

In some cases, the hydroxyl reactive groups include those such as isocyanate and epoxy. These groups can be used to form a non-cleavable covalent bond between the pendent group and the polysaccharide backbone. In these aspects, the synthesized hydrophobic derivative of the natural biodegradable polysaccharide includes chemical linkages that are enzymatically cleavable.

Other reactive groups, such as carboxyl groups, acetyl groups, or sulphate groups, are present on the ring structure of monomeric units of other natural biodegradable polysaccharides, such as chondrotin or hyaluronic acid. These groups can also be targeted for reaction with a compound having a hydrocarbon segment to be bonded to the polysaccharide backbone.

Various factors can be taken into consideration in the synthesis of the hydrophobic derivative of the natural biodegradable polysaccharide. These factors include the physical and chemical properties of the natural biodegradable polysaccharide, including its size, and the number and presence of reactive groups on the polysaccharide and solubility, the physical and chemical properties of the compound that includes the hydrocarbon segment, including its the size and solubility, and the reactivity of the compound with the polysaccharide.

In preparing the hydrophobic derivative of the natural biodegradable polysaccharide any suitable synthesis procedure can be performed. Synthesis can be carried out to provide a desired number of groups with hydrocarbon segments pendent from the polysaccharide backbone. The number and/or density of the pendent groups can be controlled, for example, by controlling the relative concentration of the compound that includes the hydrocarbon segment to the available reactive groups (e.g., hydroxyl groups) on the polysaccharide.

The type and amount of groups having the hydrocarbon segment pendent from the polysaccharide is sufficient for the hydrophobic polysaccharide to be insoluble in water. In order to achieve this, as a general approach, a hydrophobic polysaccharide is obtained or prepared wherein the groups having the hydrocarbon segment pendent from the polysaccharide backbone in an amount in the range of 0.25 (pendent group):1 (polysaccharide monomer) by weight.

The weight ratio of glucopyranose units to pendent groups can vary, but will typically be about 1:1 to about 100:1. Specifically, the weight ratio of glucopyranose units to pendent groups can be about 1:1 to about 75:1, or about 1:1 to about 50:1. Additionally, the nature and amount of the pendent group can provide a suitable degree of substitution to the polysaccharide. Typically, the degree of substitution will be in the range of about 0.1-5 or about 0.5-2.

To exemplify these levels of derivation, very low molecular weight (less than 10,000 Da) glucopyranose polymers are reacted with compounds having the hydrocarbon segment to provide low molecular weight hydrophobic glucopyranose polymers. In one mode of practice, the natural biodegradable polysaccharide maltodextrin in an amount of 10 g (MW 3000-5000 Da; -3 mmols) is dissolved in a suitable solvent, such as tetrahydrofuran. Next, a solution having butyric anhydride in an amount of 18 g (0.11 mols) is added to the maltodextrin solution. The reaction is allowed to proceed, effectively forming pendent butyrate groups on the pyranose rings of the maltodextrin polymer. This level of derivation results in a degree of substitution (DS) of butyrate group of the hydroxyl groups on the maltodextrin of about 1.

For maltodextrin and other polysaccharides that include three hydroxyl groups per monomeric unit, on average, one of the three hydroxyl groups per glycopyranose monomeric unit becomes substituted with a butyrate group. A maltodextrin polymer having this level of substitution is referred to herein as maltodextrin-butyrate DS 1. As described herein, the DS refers to the average number of reactive groups (including hydroxyl and other reactive groups) per monomeric unit that are substituted with pendent groups comprising hydrocarbon segments.

An increase in the DS can be achieved by incrementally increasing the amount of compound that provides the hydrocarbon segment to the polysaccharide. As another example, butyrylated maltodextrin having a DS of 2.5 is prepared by reacting 10 g of maltodextrin (MW 3000-5000 Da; ˜3 mmols) with 0.32 mols butyric anhydride.

The degree of substitution can influence the hydrophobic character of the polysaccharide. In turn, implants formed from hydrophobic derivatives having a substantial amount of groups having the hydrocarbon segments bonded to the polysaccharide backbone (as exemplified by a high DS) are generally more hydrophobic and can be more resistant to degradation. For example, an implant formed from maltodextrin-butyrate DS1 has a rate of degradation that is faster than an implant formed from maltodextrin-butyrate DS2.

The type of hydrocarbon segment present in the groups pendent from the polysaccharide backbone can also influence the hydrophobic properties of the polymer. In one aspect, the implant is formed using a hydrophobic polysaccharide having pendent groups with hydrocarbon segments being short chain branched alkyl group. Exemplary short chain branched alkyl group are branched C₄-C₁₀ groups. The preparation of a hydrophobic polymer with these types of pendent groups is exemplified by the reaction of maltodextrin with valproic acid/anhydride with maltodextrin (MD-val). The reaction can be carried out to provide a relatively lower degree of substitution of the hydroxyl groups, such as is in the range of 0.5-1.5. Although these polysaccharides have a lower degree of substitution, the short chain branched alkyl group imparts considerable hydrophobic properties to the polysaccharide.

Even at these low degrees of substitution the MD-val forms coatings that are very compliant and durable. Because of the low degrees of substitution, the pendent groups with the branched C₈ segment can be hydrolyzed from the polysaccharide backbone at a relatively fast rate, thereby providing a biodegradable coatings that have a relatively fast rate of degradation.

For polysaccharides having hydrolytically cleavable pendent groups that include hydrocarbon segments, penetration by an aqueous solution can promote hydrolysis and loss of groups pendent from the polysaccharide backbone. This can alter the properties of the implant, and can result in greater access to enzymes that promote the degradation of the natural biodegradable polysaccharide.

Various synthetic schemes can be used for the preparation of a hydrophobic derivative of a natural biodegradable polysaccharide. In some modes of preparation, pendent polysaccharide hydroxyl groups are reacted with a compound that includes a hydrocarbon segment and a group that is reactive with the hydroxyl groups. This reaction can provide polysaccharide with pendent groups comprising hydrocarbon segments.

Any suitable chemical group can be coupled to the polysaccharide backbone and provide the polysaccharide with hydrophobic properties, wherein the polysaccharide becomes insoluble in water. Specifically, the pendent group can include one or more atoms selected from carbon (C), hydrogen (H), oxygen (O), nitrogen (N), and sulfur (S).

In some aspects, the pendent group includes a hydrocarbon segment that is a linear, branched, or cyclic C₂-C₁₈ group. More specifically the hydrocarbon segment includes a C₂-C₁₀, or a C₄-C₈, linear, branched, or cyclic group. The hydrocarbon segment can be saturated or unsaturated, and can include alkyl groups or aromatic groups, respectively. The hydrocarbon segment can be linked to the polysaccharide chain via a hydrolyzable bond or a non-hydrolyzable bond.

In some aspects the compound having a hydrocarbon segment that is reacted with the polysaccharide backbone is derived from a natural compound. Natural compounds with hydrocarbon segments include fatty acids, fats, oils, waxes, phospholipids, prostaglandins, thromboxanes, leukotrienes, terpenes, steroids, and lipid soluble vitamins.

Exemplary natural compounds with hydrocarbon segments include fatty acids and derivatives thereof, such as fatty acid anhydrides and fatty acid halides. Exemplary fatty acids and anhydrides include acetic, propionic, butyric, isobutyric, valeric, caproic, caprylic, capric, and lauric acids and anhydrides, respectively. The hydroxyl group of a polysaccharide can be reacted with a fatty acid or anhydride to bond the hydrocarbon segment of the compound to the polysaccharide via an ester group.

The hydroxyl group of a polysaccharide can also cause the ring opening of lactones to provide pendent open-chain hydroxy esters. Exemplary lactones that can be reacted with the polysaccharide include caprolactone and glycolides.

Generally, if compounds having large hydrocarbon segments are used for the synthesis of the hydrophobic derivative, a smaller amount of the compound may be needed for its synthesis. For example, as a general rule, if a compound having a hydrocarbon segments with an alkyl chain length of Cₓ is used to prepare a hydrophobic derivative with a DS of 1, a compound having a hydrocarbon segment with an alkyl chain length of C(_{x×2}) is reacted in an amount to provide a hydrophobic derivative with a DS of 0.5.

The hydrophobic derivative of the natural biodegradable polysaccharide can also be synthesized having combinations of pendent groups with two or more different hydrocarbon segments, respectively. For example, the hydrophobic derivative can be synthesized using compounds having hydrocarbon segments with different alkyl chain lengths. In one mode of practice, a polysaccharide is reacted with a mixture of two or more fatty acids (or derivatives thereof) selected from the group of acetic acid, propionic acid, butyric acid, isobutyric acid, valeric acid, caproic acid, caprylic acid, capric acid, and lauric acid to generate the hydrophobic derivative.

In other cases the hydrophobic derivative is synthesized having a non-hydrolyzable bond linking the hydrocarbon segment to the polysaccharide backbone. Exemplary non-hydrolyzable bonds include urethane bonds.

The hydrophobic derivative of the natural biodegradable polysaccharide can also be synthesized so that hydrocarbon segments are individually linked to the polysaccharide backbone via both hydrolyzable and non-hydrolyzable bonds. As another example, a hydrophobic derivative is prepared by reacting a mixture of butyric acid anhydride and butyl isocyanate with maltodextrin. This yields a hydrophobic derivative of maltodextrin with pendent butyric acid groups that are individually covalently bonded to the maltodextrin backbone with hydrolyzable ester linkages and non-hydrolyzable urethane linkages. The degradation of a coating having this type of hydrophobic derivative can occur by loss of the butyrate groups from hydrolysis of the ester linkages. However, a portion of the butyrate groups (the ones that are bonded via the urethane groups) are not removed from the polysaccharide backbone and therefore the natural biodegradable polysaccharide can maintain a desired degree of hydrophobicity, prior to enzymatic degradation of the polysaccharide backbone.

In some aspects, the group that is pendent from the polysaccharide backbone has properties of an active pharmaceutical ingredient (API). In this regard, the implants include polysaccharide-coupled API. In some aspects, an API which has a hydrocarbon segment can be hydrolyzed from the natural biodegradable polymer and released from the matrix to provide a therapeutic effect. One example of a therapeutically useful compound having a hydrocarbon segments is butyric acid, which has been shown to elicit tumor cell differentiation and apoptosis, and is thought to be useful for the treatment of cancer and other blood diseases.

Other illustrative compounds that include hydrocarbon segments include valproic acid and retinoic acid. These compounds can be coupled to a polysaccharide backbone to provide a pendent group, and then cleaved from the polysaccharide backbone upon degradation of the implant in vivo. Retinoic acid is known to possess antiproliferative effects and is thought to be useful for treatment of proliferative vitreoretinopathy (PVR). The pendent group that provides a therapeutic effect can also be a natural compound (such as butyric acid, valproic acid, and retinoic acid).

Another illustrative class of compounds that can be coupled to the polysaccharide backbone is the corticosteroids. An exemplary corticosteroid is triamcinolone. One method of coupling triamcinolone to a natural biodegradable polymer is by employing a modification of the method described in Cayanis, E. et al., Generation of an Auto-anti-idiotypic Antibody that Binds to Glucocorticoid Receptor, The Journal of Biol. Chem., 261(11): 5094-5103 (1986). Triamcinolone hexanoic acid is prepared by reaction of triamcinolone with ketohexanoic acid; an acid chloride of the resulting triamcinolone hexanoic acid can be formed and then reacted with the natural biodegradable polymer, such as maltodextrin or polyalditol, resulting in pendent triamcinolone groups coupled via ester bonds to the natural biodegradable polymer.

The hydrophobic derivative of the natural biodegradable polysaccharide can also be synthesized having two or more different pendent groups, wherein at least one of the pendent groups includes an API. The hydrophobic polysaccharide can be synthesized with an amount of a pendent groups including an API, that when released from the polysaccharide, provides a therapeutic effect to the subject. An example of such a hydrophobic derivative is maltodextrin-caproate-triamcinolone. This hydrophobic derivative can be prepared by reacting a mixture including triamcinolone hexanoic acid and an excess of caproic anhydride (n-hexanoic anhydride) with maltodextrin to provide a derivative with a DS of 2.5.

In some aspects, the group that is pendent from the polysaccharide includes a hydrocarbon segment that is an aromatic group, such as a phenyl group. As one example, o-acetylsalicylic acid is reacted with a polysaccharide such as maltodextrin to provide pendent chemical group having a hydrocarbon segment that is a phenyl group, and a non-hydrocarbon segment that is an acetate group wherein the pendent group is linked to the polysaccharide via an ester bond.

Additional features and descriptions of the biodegradable polymers that include the hydrophobic derivatives of natural biodegradable polysaccharides can be found, for example, in U.S. Patent Publication Nos. 2007/0218102, 2007/0260054 and 2007/0224247, and references cited therein.

### Linking Group

A pendant group (PG) can optionally be linked to a suitable linker or linking group (L), and the suitable linking group (L) can then be linked to a monosaccharide unit (M), to provide the polysaccharide. As such, a pendant group (PG) can independently be absent or present on each one of the monosaccharide units (M) of the polysaccharide. Additionally, when more than one pendant group (PG) is present on the polysaccharide, each of the pendant groups (PG) can be the same, or can be different, from the other pendant groups (PG) present on the polysaccharide.

As shown herein (see, e.g., Table I below), the reactive functional groups present on the pendant group (PG) and monosaccharide unit (M) will typically influence the requisite functional groups to be present on the linking group (L). The nature of the linking group (L) is not critical, provided the pendant group (PG) employed possesses acceptable mechanical properties and release kinetics for the selected therapeutic application. The or linking group (L) is typically a divalent organic radical having a molecular weight of from about 25 daltons to about 400 daltons. More specifically, the linking group (L) can have a molecular weight of from about 40 daltons to about 200 daltons.

The resulting linking group (L), present on the polysaccharide, can be biologically inactive, or can itself possess biological activity. The linking group (L) can also include other functional groups (including hydroxy groups, mercapto groups, amine groups, carboxylic acids, as well as others) that can be used to modify the properties of the polysaccharide (e.g. for appending other molecules to monosaccharide unit (M)), for changing the solubility of the polysaccharide, or for effecting the biodistribution of the polysaccharide.

Specifically, the linking group (L) can be a divalent, branched or unbranched, saturated or unsaturated, hydrocarbon chain, having from 1 to 50 carbon atoms, wherein one or more (e.g. 1, 2, 3, or 4) of the carbon atoms is optionally interrupted with, e.g., one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO), sulfonyl (SO₂) or (—NR—), wherein R can be hydrogen, alkyl, cycloalkyl alkyl, or aryl alkyl.

The hydrocarbon chain of the linking group (L) can optionally be substituted on carbon with one or more (e.g. 1, 2, 3, or 4) substituents selected from the group of alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

In one specific embodiment of the presently disclosed subject matter, the linking group can be lipophillic (hydrophobic). In another specific embodiment of the presently disclosed subject matter, the linking group can be hydrophilic (lipophobic).

TABLE I
Reactive functional groups present on the pendant group (PG) and
monosaccharide unit (M); and resulting linkage

| Functional Group on | | |
|---|---|---|
| Pendant Group (PG)/ | Functional Group on | |
| Monosaccharide Unit (M) | Linking Group (L) | Resulting Linkage |
| —COOH | —OH | Carboxylic Ester |
| —COOH | —NH₂ | Amide |
| —COOH | —SH | Thioester |
| —OH | —COOH | Carboxylic Ester |
| —SH | —COOH | Thioester |
| —NH₂ | —COOH | Amide |
| —OH | —OP(═O)(OH)₂ | Phosphoric Acid Ester |
| —OH | —OP(═O)(OR)₂ | Phosphoric Acid Ester |
| —OH | —SO₂OH | Sulphonic Acid Ester |
| —OH | —OC(O)X (wherein | Carbonate |
| | X can be a suitable | |
| | leaving group, such | |
| | as halogen) | |
| —OH | —OB(OR)₂ | Borate |
| —OH | —OSi(OR)₃ or | Silyl ether |
| | —OSi(CH)₂OR | |

Specifically, the linking group (L) can be a divalent, branched or unbranched, saturated or unsaturated, hydrocarbon chain, having a length of about 1 Angstrom to about 500 Angstroms, about 1 Angstrom to about 250 Angstroms or about 1 Angstrom to about 100 Angstroms.

### Active Pharmaceutical Ingredient (API)

The active pharmaceutical ingredient (API) or active agent will be substantially insoluble in the biocompatible solvent system, such that the API will typically be undissolved, unsolubilized and/or suspended in the formulation. For example, the API can have a solubility of less than about 10 g/L in the biocompatible solvent system, at 25° C. and 1 atm. Specifically, the API can have a solubility of less than about 1 g/L, less than about 500 mg/L, less than about 250 mg/L, less than about 100 mg/L, less than about 50 mg/L or less than about 10 mg/L, in the biocompatible solvent system, at 25° C. and 1 atm. The solubility of the API can be measured in water as well. For example, the API can have a water solubility of greater than about 500 mg/L, greater than about 1 g/L, greater than about 5 g/L, greater than about 10 g/L, greater than about 20 g/L, greater than about 25 g/L, greater than about 50 g/L at 25° C. and 1 atm, greater than about 100 g/L at 25° C. and 1 atm, or greater than about 250 g/L at 25° C. and 1 atm. In specific embodiments, the API is hydrophilic.

Selection of an active pharmaceutical ingredient (API) that is substantially insoluble in the biocompatible solvent system can provide a composition in which the API is dispersed throughout the composition. Such a dispersion can be a uniform or substantially uniform dispersion of the API throughout the composition. Upon forming an implant in vivo, the active pharmaceutical ingredient (API) can be dispersed (e.g., uniformly or substantially uniformly) throughout the implant. The active pharmaceutical ingredient (API) can therefore be released to the subject over a period of time, thus providing for delivery of the active pharmaceutical ingredient (API) with a controlled burst of active pharmaceutical ingredient (API) and sustained release thereafter. In one embodiment, the implant delivers an effective amount of active pharmaceutical ingredient (API) in a sustained, zero-order release profile.

Specific suitable classes of API include, e.g., macromolecules, proteins, peptides, genes, polynucleotides and analogues thereof, nucleotides, biological agents, small molecules, and complexes thereof.

Specific suitable APIs include, for example, those APIs illustrated in Table 2 below. Additional suitable APIs include, for example, those APIs illustrated in the Physician's Desk Reference 64^{th} Edition (2010). It is appreciated that those of skill in the art of pharmaceutical chemistry understand that for many of the APIs illustrated in Table 2 below, that the proprietary name is provided, but that reference is made to the API. For example, the API “aspirin” is intended to refer to the compound acetylsalicyclic acid.

TABLE 2
Indications and APIs for Injectable Drug Delivery System

| | Disease/Drug | | |
|---|---|---|---|
| Broad Disease | Categories from | | |
| Categories | the PDR | Specific Indications | APIs |
| Autoimmune | Immuno- | Cryopyrin-associated | Rilonacept |
| Diseases | suppressants | periodic syndromes | |
| | | (CAPS) | |
| | | Familial cold auto- | Rilonacept |
| | | inflammatory syndrome | |
| | | (FCAS) | |
| | | Muckle-wells | Rilonacept |
| | | syndrome (MWS) | |
| | | Mild to moderate atopic | Pimecrolimus |
| | | dermatitis | |
| | | Kidney, liver and heart | Cyclosporine |
| | | transplantation | |
| | | Rheumatoid arthritis | Cyclosporine |
| | | Psoriasis | Cyclosporine |
| | Multiple sclerosis | Multiple Sclerosis | Interferon beta-1b |
| | | Multiple Sclerosis | Interferon beta-1a |
| | | Relapsing forms of | Moxifloxacin hydrochloride |
| | | multiple sclerosis | |
| Neurological | Alzheimer's | Alzheimer's, mild to | Donepezil hydrochloride |
| Diseases | Disease | moderate, as well as | |
| | Management | severe Alzheimer's | |
| | | Parkinson's Disease | Rivastigmine tartrate |
| | | dementia | |
| | Amyotrophic | Amyotrophic lateral | Riluzole |
| | Lateral Sclerosis | sclerosis (ALS) | |
| | Therapeutic | | |
| | Agents | | |
| | Anticonvulsants | Sedation | Pentobarbital sodium |
| | | Short-term treatment of | Pentobarbital sodium |
| | | insomnia | |
| | | Preanesthesia | Pentobarbital sodium |
| | | Acute convulsive | Pentobarbital sodium |
| | | episodes | |
| | | Panic disorder | Clonazepam |
| | | Anxiety disorders | Diazepam |
| | | Muscle spasms | Diazepam |
| | | Neuropathic pain | Pregabalin |
| | | associated with diabetic | |
| | | peripheral neuropathy | |
| | | Fibromyalgia | Pregabalin |
| | | Postherpetic neuralgia | Pregabalin |
| | | Partial onset seizures | Pregabalin |
| | | Infantile spasms | Vigabatrin |
| | | Seizures, during or after | Phenytoin sodium |
| | | neurosurgery | |
| | | Mania | Divalproex sodium |
| | | Epilepsy | Divalproex sodium |
| | | Migraine | Divalproex sodium |
| | | Bipolar disorder | Lamotrigine |
| | Antiparkinsonian | Idiopathic Parkinson's | Entacapone |
| | Agents | Disease | |
| | | Parkinson's Disease | Ropinirole |
| | | Restless leg syndrome | Ropinirole |
| | Central nervous | Narcolepsy | Dextroamphetamine sulfate |
| | system stimulants | Attention deficit | Dextroamphetamine sulfate |
| | | disorder with | |
| | | hyperactivity | |
| | | Improve wakefulness | Armodafinil |
| | | due to sleep disorders | |
| | Cholinesterase | Dementia of the | Donepezil hydrochloride |
| | inhibitors | Alzheimer's type | |
| | | Dementia associated | Rivastigmine tartrate |
| | | with Parkinson's | |
| | | Disease | |
| | Dopamine | Parkinson's disease | Ropinirole |
| | receptor agonists | Restless leg syndrome | Ropinirole |
| | | Chorea due to | Tetrabenazine |
| | | Huntington's disease | |
| | Psychotherapeutic | Anxiety disorders | Diazepam |
| | agents | Acute agitation | Diazepam |
| | | Tremor | Diazepam |
| | | Impending or acute | Diazepam |
| | | delirium tremens and | |
| | | hallucinations | |
| | | Skeletal muscle spasms | Diazepam |
| | | Major depressive | Duloxetine hydrochloride |
| | | disorder | |
| | | Diabetic peripheral | Duloxetine hydrochloride |
| | | neuropathic pain | |
| | | Fibromyalgia | Duloxetine hydrochloride |
| | | Panic disorder | Paroxetine hydrochloride |
| | | Social anxiety disorder | Paroxetine hydrochloride |
| | | Premenstrual dysphoric | Paroxetine hydrochloride |
| | | disorder | |
| | | Posttraumatic stress | Paroxetine hydrochloride |
| | | disorder | |
| | | Obsessive compulsive | Fluoxetine hydrochloride |
| | | disorder | |
| | | Bulimia nervosa | Fluoxetine hydrochloride |
| | | Depressive episodes | Olanzapine and fluoxetine |
| | | associated with Bipolar | hydrochloride |
| | | I disorder | |
| | | Treatment Resistant | Olanzapine and fluoxetine |
| | | Depression | hydrochloride |
| | | Schizophrenia | Thioridazine hydrochloride |
| | | Seizure disorders | Clonazepam |
| | | Bipolar mania | Ziprasidone hydrochloride |
| | | Acute agitation in | Ziprasidone hydrochloride |
| | | schizophrenia patients | |
| | | Schizoaffective | Paliperidone |
| | | disorder | |
| | | Epilepsy | Divalproex sodium |
| | | Migraine | Divalproex sodium |
| | | Attention deficit | Guanfacine |
| | | hyperactivity disorder | |
| Pain | Analgesics | Common cold | Acetaminophen |
| | | Headache | Acetaminophen |
| | | Backache | Acetaminophen |
| | | Arthritis | Acetaminophen |
| | | Toothache | Acetaminophen |
| | | Muscular aches | Acetaminophen |
| | | Premenstrual and | Acetaminophen |
| | | menstrual cramps | |
| | | Fever | Acetaminophen |
| | | Pain, mild to | Acetaminophen and codeine |
| | | moderately severe | phosphate |
| | | Flu | Acetaminophen |
| | | Sore throat | Acetaminophen |
| | | Major depressive | Duloxetine hydrochloride |
| | | disorder | |
| | | Anxiety disorder | Duloxetine hydrochloride |
| | | Diabetic peripheral | Duloxetine hydrochloride |
| | | neuropathic pain | |
| | | Fibromyalgia | Duloxetine hydrochloride |
| | | Post herpetic neuralgia | Pregabalin |
| | | Partial onset seizures | Pregabalin |
| | | Pain, moderate to | Tramadol hydrochloride |
| | | moderately severe | |
| | | chronic pain | |
| | | Epilepsy | Carbamazepine |
| | | Trigeminal neuralgia | Carbamazepine |
| | | Osteoarthritis | Sodium hyaluronate |
| | | Sprains | Methyl salicylate |
| | | Strains | Methyl salicylate |
| | | Bruises | Methyl salicylate |
| | | Pain, moderate to | Morphine sulfate and naltrexone |
| | | moderately severe | hydrochloride |
| | | chronic pain | |
| | | Pain | Hydromorphone hydrochloride |
| | | Pain, break-through in | Fentanyl |
| | | cancer patients | |
| | | Pain, moderate to | Oxymorphone hydrochloride |
| | | severe | |
| | | Rheumatoid arthritis | Naproxen |
| | | Osteoarthritis | Naproxen |
| | | Ankylosing spondylitis | Naproxen |
| | | Tendonitis | Naproxen |
| | | Bursitis | Naproxen |
| | | Gout | Naproxen |
| | | Primary dysmenorrhea | Naproxen |
| | | Juvenile Rheumatoid | Celecoxib |
| | | arthritis | |
| | | Familial adenomatous | Celecoxib |
| | | polyposis (FAP) | |
| | | Acute painful shoulder | Sulindac |
| | | Gouty arthritis | Sulindac |
| | | Contusions | Diclofenac epolamine topical |
| | | | patch |
| | | Patent ductus arteriosus | Indomethacin |
| | | Migraines | Sumatriptan and naproxen |
| | | | sodium |
| | | Vascular indications | Aspirin |
| | | (ischemic stroke, TIA, | |
| | | acute MI, prevention of | |
| | | recurrent MI, unstable | |
| | | angina pectoris, chronic | |
| | | stable angina pectoris) | |
| | | Revascularization | Aspirin |
| | | procedures (coronary | |
| | | artery bypass graft | |
| | | (CABG), percutaneous | |
| | | transluminal coronary | |
| | | angioplasty (PTCA) | |
| | | and carotid | |
| | | endarterectomy) | |
| | | Rheumatologic disease | Aspirin |
| | | indications (rheumatoid | |
| | | arthritis, juvenile | |
| | | rheumatoid arthritis, | |
| | | spondyloarthropathies, | |
| | | osteoarthritis, and the | |
| | | arthritis and pleurisy of | |
| | | systemic lupus | |
| | | erythematosis (SLE) | |
| | | Hyperuricemia in | Febuxostat |
| | | patients with gout | |
| | Migraine | Mania | Divalproex sodium |
| | preparations | Epilepsy | Divalproex sodium |
| | | Migraine | Divalproex sodium |
| | | Cluster headaches | Sumatriptan succinate |
| | | Muscle spasms | Cyclobenzaprine hydrochloride |
| | | Painful musculoskeletal | Metaxalone |
| | | conditions | |
| | Analgesics and | Mild to moderate | Methyl salicylate |
| | combinations | muscle aches and joint | |
| | | pain | |
| | | Pain associated with | Lidocaine |
| | | post-herpetic neuralgia | |
| | | Bladder pain or | Pentosan polysulfate sodium |
| | | discomfort associated | |
| | | with interstitial cystitis | |
| | Nonsteroidal | Rheumatoid arthritis | Naproxen |
| | anti- | Osteoarthritis | Naproxen |
| | inflammatory | Ankylosing spondylitis | Naproxen |
| | drugs (NSAIDS) | Tendonitis | Naproxen |
| | & combinations | Bursitis | Naproxen |
| | | Gout | Naproxen |
| | | Primary dysmenorrhea | Naproxen |
| | | Pain | Naproxen |
| | | Juvenile Rheumatoid | Celecoxib |
| | | arthritis | |
| | | Familial adenomatous | Celecoxib |
| | | polyposis (FAP) | |
| | | Acute painful shoulder | Sulindac |
| | | Osteoarthritis | Celecoxib |
| | | Rheumatoid arthritis | Celecoxib |
| | | Ankylosing spondylitis | Celecoxib |
| | | Pain, acute | Celecoxib |
| | | Primary dysmenorrhea | Celecoxib |
| | | Gouty arthritis | Sulindac |
| | | Contusions | Diclofenac epolamine |
| | | Strains | Diclofenac epolamine |
| | | Sprains | Diclofenac epolamine |
| | | Patent ductus arteriosus | Indomethacin |
| | | in premature infants | |
| | | Migraines | Sumatriptan and naproxen |
| | | | sodium |
| | | Vascular indications | Aspirin |
| | | (ischemic stroke, TIA, | |
| | | acute MI, prevention of | |
| | | recurrent MI, unstable | |
| | | angina pectoris, chronic | |
| | | stable angina pectoris) | |
| | | Revascularization | Aspirin |
| | | procedures (coronary | |
| | | artery bypass graft | |
| | | (CABG), percutaneous | |
| | | transluminal coronary | |
| | | angioplasty (PTCA) | |
| | | and carotid | |
| | | endarterectomy) | |
| | | Rheumatologic disease | Aspirin |
| | | indications (rheumatoid | |
| | | arthritis, juvenile | |
| | | rheumatoid arthritis, | |
| | | spondyloarthropathies, | |
| | | osteoarthritis, and the | |
| | | arthritis and pleurisy of | |
| | | systemic lupus | |
| | | erythematosis (SLE) | |
| | | Minor aches and pains | Aspirin |
| | | Common cold | Ibuprofen |
| | | Headache | Ibuprofen |
| | | Backache | Ibuprofen |
| | | Arthritis, minor pain | Ibuprofen |
| | | Toothache | Ibuprofen |
| | | Muscular aches | Ibuprofen |
| | | Menstrual cramps | Ibuprofen |
| | | Fever | Ibuprofen |
| | | Pain, moderate to | Oxycodone and aspirin |
| | | moderately severe | |
| | Antihistamines & | Seasonal Allergic | Fexofenadine hydrochloride |
| | Combinations | Rhinitis | |
| | | Chronic Idiopathic | Fexofenadine hydrochloride |
| | | Urticaria | |
| | | Hay Fever or other | Diphenhydramine hydrochloride |
| | | upper respiratory | |
| | | allergies | |
| | | Common cold | Diphenhydramine hydrochloride |
| | | Perennial Allergic | Desloratadine |
| | | Rhinitis | |
| | | Hives | Cetirizine hydrochloride |
| | | Itchy eyes due to | Ketotifen |
| | | pollen, ragweed, grass, | |
| | | animal hair and dander | |
| | | Swelling of nasal | Cetirizine hydrochloride and |
| | | passages | pseudoephedrine hydrochloride |
| | | Sinus congestion and | Cetirizine hydrochloride and |
| | | pressure | pseudoephedrine hydrochloride |
| | | Freer breathing through | Cetirizine hydrochloride and |
| | | the nose | pseudoephedrine hydrochloride |
| | Decongestant and | Seasonal allergic | Fexofenadine hydrochloride and |
| | combinations | allergies | pseudoephedrine hydrochloride |
| | | Common cold | Pseudoephedrine hydrochloride |
| | | Hay fever | Pseudoephedrine hydrochloride |
| | | Swelling of nasal | Pseudoephedrine hydrochloride |
| | | passages | |
| | | Sinus pressure | Pseudoephedrine hydrochloride |
| Inflammatory | Anti- | Atopic dermatitis | Pimecrolimus |
| Diseases | Inflammatory | Dermatoses | Mometasone furoate |
| | agents | (inflammatory/pruritic | |
| | | manifestations of | |
| | | corticosteroid- | |
| | | responsive) | |
| | | Rosacea, mild/moderate | Azelaic acid |
| | | Allergic conjunctivitis | Epinastine hydrochloride |
| | | Maintenance of | Mesalamine |
| | | remission of ulcerative | |
| | | colitis | |
| | | Moderate to severe | Tacrolimus |
| | | atopic dermatitis | |
| | | Adult patients with | Golimumab |
| | | active psoriatic arthritis | |
| | | Adult patients with | Golimumab |
| | | active ankylosing | |
| | | spodylitis | |
| | | Juvenile idiopathic | Abatacept |
| | | arthritis | |
| | | Erythema nodosum | Thalidomide |
| | | leprosum | |
| | | Management of | Febuxostat |
| | | hyperuricemia in | |
| | | patients with gout | |
| | Steroidal anti- | Seasonal or perennial | Beclomethasone dipropionate, |
| | inflammatory | allergies | monohydrate |
| | agents | Prevention of | Beclomethasone dipropionate, |
| | | recurrence of nasal | monohydrate |
| | | polyps following | |
| | | surgical removal | |
| | | Acute otitis externa | Ciprofloxacin and |
| | | | dexamethasone |
| | Steroidal anti- | Asthma | Fluticasone propionate and |
| | inflammatory | | salmeterol |
| | agents and | Airflow obstruction and | Fluticasone propionate and |
| | combinations | reducing exacerbations | salmeterol |
| | | (chronic obstructive | |
| | | pulmonary disease | |
| | | (COPD)) | |
| | Steroids and | Pruritic manifestations | Betamethasone |
| | combinations | of corticosteroid- | |
| | | responsive dermatoses | |
| | | Symptomatic tinea | Clotrimazole and betamethasone |
| | | pedis | dipropionate |
| | | Symptomatic tinea | Clotrimazole and betamethasone |
| | | cruris | dipropionate |
| | | Symptomatic tinea | Clotrimazole and betamethasone |
| | | corporis | dipropionate |
| | | Pruritic manifestations | Betamethasone valerate |
| | | of corticosteroid- | |
| | | responsive dermatoses | |
| | | of the scalp | |
| | | Diaper dermatitis | Miconazole nitrate |
| | Anti-Rheumatic | Rheumatoid arthritis | Naproxen |
| | agents | Osteoarthritis | Naproxen |
| | | Ankylosing spondylitis | Naproxen |
| | | Juvenile arthritis | Naproxen |
| | | Tendonitis | Naproxen |
| | | Bursitis | Naproxen |
| | | Gout | Naproxen |
| | | Pain | Naproxen |
| | | Primary dysmenorrhea | Naproxen |
| | | Moderately to severely | Interleukin-1 receptor |
| | | active rheumatoid | anatagonist (IL-1Ra) |
| | | arthritis | |
| | | Adult rheumatoid | Abatacept |
| | | arthritis | |
| | | Adult patients with | Golimumab |
| | | moderately to severely | |
| | | active rheumatoid | |
| | | arthritis | |
| | | Rheumatoid arthritis | Rituximab |
| | | Vascular indications | Aspirin |
| | | (Ischemic Stroke, TIA, | |
| | | Acute MI, prevention of | |
| | | recurrent MI, unstable | |
| | | angina pectoris, chronic | |
| | | stable angina pectoris | |
| | | Revascularization | Aspirin |
| | | procedures (coronary | |
| | | artery bypass graft, | |
| | | percutaneous | |
| | | transluminal coronary | |
| | | angioplasty, carotid | |
| | | endarterectomy) | |
| | | Familial adenomatous | Celecoxib |
| | | polyposis | |
| | | Gouty arthritis | Sulindac |
| | | Crohn's disease | Certolizumab pegol |
| | | Psoriatic arthritis | Etanercept |
| | | Plaque psoriasis | Etanercept |
| | | Kidney, liver and heart | Cyclosporine |
| | | transplantation | |
| | | Psoriasis | Cyclosporine |
| | | Ulcerative colitis | Infliximab |
| | Clotting disorders | Deep vein thrombosis | Fondaparinux sodium |
| | | Ischemic complications | Dalteparin sodium |
| | | in unstable angina and | |
| | | non-Q-wave | |
| | | myocardial infarction | |
| | | Symptomatic venous | Dalteparin sodium |
| | | thromboembolism | |
| | | Stroke associated with | Aspirin and dipyridamole |
| | | transient ischemia of | |
| | | the brain | |
| | | Ischemic stroke due to | Aspirin and dipyridamole |
| | | thrombosis | |
| | | Acute coronary | Prasurgrel |
| | | syndrome | |
| | | Primary pulmonary | Epoprostenol sodium |
| | | hypertension | |
| | | Primary pulmonary | Epoprostenol sodium |
| | | hypertension associated | |
| | | with scleroderma | |
| | | spectrum | |
| | | Acute coronary | Eptifibatide |
| | | syndrome | |
| | | Unstable angina | Bivalirudin |
| | | (undergoing | |
| | | percutaneous | |
| | | transluminal coronary | |
| | | angioplasty) | |
| | | Prevention of peri- | Antithrombin (recombinant) |
| | | operative and per- | |
| | | partum thromboembolic | |
| | | events | |
| | | Heparin-induced | Lepirudin (rDNA) |
| | | thrombocytopenia and | |
| | | associated | |
| | | thromboembolic | |
| | | disease | |
| | | Acute myocardial | Alteplase |
| | | infarction | |
| | | Acute ischemic stroke | Alteplase |
| | | Pulmonary embolism | Alteplase |
| Cancer | Antineoplastics | Chemotherapy-induced | Palonosetron hydrochloride |
| | | nausea and vomiting | |
| | | Postoperative nausea | Palonosetron hydrochloride |
| | | and vomiting | |
| | | Prevention of | Dolasetron mesylate |
| | | ostoperative nausea and | |
| | | vomiting | |
| | | Anemia with chronic | Darbepoetin alfa |
| | | renal failure | |
| | | Anemia with non- | Darbepoetin alfa |
| | | myeloid malignancies | |
| | | due to chemotherapy | |
| | | Initial management of | Rasburicase |
| | | plasma uric acid levels | |
| | | in pediatric patients | |
| | | w/leukemia lymphoma, | |
| | | solid tumor | |
| | | malignancies | |
| | | Anemia, chronic renal | Epoetin alfa |
| | | failure | |
| | | Anemia, Cancer | Epoetin alfa |
| | | Anemia in zidovudine- | Epoetin alfa |
| | | treated HIV-infected | |
| | | patients | |
| | | Oral mucositis in | Palifermin |
| | | patients w/hematologic | |
| | | malignancies | |
| | | Reduction of allogeneic | Epoetin alfa |
| | | blood transfusions in | |
| | | surgery patients | |
| | | Hypothyroidism | Levothyroxine sodium |
| | | Pituitary TSH | Levothyroxine sodium |
| | | suppression | |
| | | Decrease incidence of | Pegfilgrastim |
| | | infection as manifested | |
| | | by febrile neutropenia | |
| | | (receiving | |
| | | myelosuppressive | |
| | | anticancer drugs) | |
| | | Patients with acute | Filgrastim |
| | | myeloid leukemia | |
| | | receiving induction or | |
| | | consolidation | |
| | | chemotherapy | |
| | | Cancer patients | Filgrastim |
| | | receiving bone marrow | |
| | | Patients undergoing | Filgrastin |
| | | peripheral blood | |
| | | progenitor cell | |
| | | collection and therapy | |
| | | Severe chronic | Filgrastin |
| | | neutropenia | |
| | | Reduces neurologic | Mitoxantrone |
| | | disability/frequency of | |
| | | clinical relapse re | |
| | | Multiple Sclerosis | |
| | | Acromegaly | Octreotide acetate |
| | | Cardinoid tumors | Octreotide acetate |
| | | Vasoactive intestinal | Octreotide acetate |
| | | peptide tumors | |
| | | (VIPomas) | |
| | | Secondary | Cinacalcet |
| | | hyperparathyroidism in | |
| | | patients with chronic | |
| | | kidney disease on | |
| | | dialysis | |
| | | Hypercalcemia in | Cinacalcet |
| | | patients with | |
| | | parathyroid carcinoma | |
| | | Nausea/vomiting re | Ondansetron hydrochloride |
| | | radiotherapy | |
| | | Hypercalcemia of | Zoledronic acid |
| | | malignancy | |
| | | Multiple myeloma and | Zoledronic acid |
| | | bone metastases of | |
| | | solid tumors | |
| | | CD25-directed | Altretamine |
| | | cytotoxin T-cell | |
| | | lymphoma | |
| | | A single agent in | Altretamine |
| | | palliative treatment of | |
| | | patients with | |
| | | persistent/recurrent | |
| | | ovarian cancer | |
| | | following 1^{st} line | |
| | | therapy w/cisplatin | |
| | | and/or alkylating agent- | |
| | | based combination | |
| | | Palliative treatment of | Busulfan |
| | | chronic myelogenous | |
| | | leukemia | |
| | | Newly diagnosed | Temozolomide |
| | | glioblastoma | |
| | | multiforme | |
| | | Anaplastic astrocytoma | Temozolomide |
| | | (refractory) | |
| | | Palliative treatment of | Melphalan hydrochloride |
| | | patients with multiple | |
| | | myeloma and oral | |
| | | therapy is not | |
| | | appropriate | |
| | | Palliative of non- | Melphalan |
| | | resectable epithelial | |
| | | carcinoma of the ovary | |
| | | Lymphatic leukemia | Chlorambucil |
| | | Malignant lymphomas | Chlorambucil |
| | | Giant follicular | Chlorambucil |
| | | lymphoma, including | |
| | | lymphosarcoma | |
| | | Hodgkin's disease | Mechlorethamine hydrochloride |
| | | (stages III and IV) | |
| | | Chronic myelocytic or | Mechlorethamine hydrochloride |
| | | chronic lymphocytic | |
| | | leukemia | |
| | | Polycythemia vera | Mechlorethamine hydrochloride |
| | | Mycosis fungoides | Mechlorethamine hydrochloride |
| | | Bronchogenic | Mechlorethamine hydrochloride |
| | | carcinoma | |
| | | Non-Hodgkin's | Bendamustine hydrochloride |
| | | lymphoma | |
| | | Newly-diagnosed | Polifeprosan 20 and carmustine |
| | | high-grade malignant | |
| | | glioma patents (adjunct | |
| | | to surgery/radiation) | |
| | | Glioblastoma | Polifeprosan 20 and carmustine |
| | | multiforme patients | |
| | | (recurrent - adjunct to | |
| | | surgery) | |
| | | Wilms' tumor | Dactinomycin |
| | | Childhood | Dactinomycin |
| | | rhabdomyosarcoma | |
| | | Ewing's sarcoma and | Dactinomycin |
| | | metastatic | |
| | | Nonseminomatous | Dactinomycin |
| | | testicular cancer | |
| | | Multiple myeloma | Doxorubicin hydrochloride |
| | | Urinary bladder BCG- | Valrubicin |
| | | refractory carcinoma | |
| | | Nonsquamous non- | Pemetrexed disodium |
| | | small cell lung cancer - | |
| | | combination with | |
| | | cisplatin | |
| | | Nonsquamous non- | Pemetrexed disodium |
| | | small cell lung cancer - | |
| | | maintenance | |
| | | Nonsquamous non- | Pemetrexed disodium |
| | | small cell lung cancer - | |
| | | after prior | |
| | | chemotherapy | |
| | | Mesothelioma | Pemetrexed disodium |
| | | Relapsed or refractory | Clofarabine |
| | | acute lymphoblastic | |
| | | leukemia (pediatric | |
| | | patients 1-21) after at | |
| | | least 2 prior regimens | |
| | | Acute hairy cell | Cladribine |
| | | leukemia | |
| | | Acute nonlymphocytic | Thioguanine |
| | | leukemia | |
| | | Colorectal cancer | Capecitabine |
| | | Breast cancer | Capecitabine |
| | | Cutaneous T-cell | Denileukin diftitox |
| | | lymphoma | |
| | | Vasomotor symptoms | Conjugated estrogens |
| | | (moderate/severe) due | |
| | | to menopause | |
| | | Vulvar and vaginal | Conjugated estrogens |
| | | atrophy symptoms | |
| | | (moderate/severe) due | |
| | | to menopause | |
| | | Hypoestrogenism due | Conjugated estrogens |
| | | to hypogonadism | |
| | | Advanced androgen- | Conjugated estrogens |
| | | dependent carcinoma of | |
| | | the prostrate | |
| | | Osteoporosis | Conjugated estrogens |
| | | (prevention) | |
| | | Prostate cancer | Leuprolide acetate |
| | | (advanced) | |
| | | Renal cell carcinoma | Everolimus |
| | | (advanced) | |
| | | CD20 antigen- | Tositumomab |
| | | expressing relapsed or | |
| | | refractory, low grade, | |
| | | follicular, or | |
| | | transformed non- | |
| | | Hodgkin's lymphoma | |
| | | Myelodysplastic | Decitabine |
| | | syndromes (MDS) | |
| | | Colon cancer (stage III) | Oxaliplatin |
| | | Acute lymphoblastic | Asparagine |
| | | leukemia (ALL) | |
| | | Pancreatic cancer | Gemcitabine hydrochloride |
| | | Ovarian cancer | Gemcitabine hydrochloride |
| | | Philadelphia | Imatinib mesylate |
| | | chromosome positive | |
| | | chronic myeloid | |
| | | leukemia | |
| | | Philadelphia | Imatinib mesylate |
| | | chromosome positive | |
| | | chronic myeloid | |
| | | leukemia in blast crisis | |
| | | accelerated/chronic | |
| | | phase | |
| | | Philadelphia | Imatinib mesylate |
| | | chromosome positive | |
| | | chronic myeloid | |
| | | leukemia (pediatric) | |
| | | Philadelphia | Imatinib mesylate |
| | | chromosome positive | |
| | | acute lymphoblastic | |
| | | leukemia | |
| | | Myelodysplastic/ | Imatinib mesylate |
| | | myeloproliferative | |
| | | diseases associated with | |
| | | PDGR | |
| | | Aggressive systemic | Imatinib mesylate |
| | | mastocytosis with the | |
| | | D816V c-Kit mutation | |
| | | Hypereosinophilic | Imatinib mesylate |
| | | syndrome and/or | |
| | | chronic eosinohpilic | |
| | | leukemia | |
| | | Unresectable, recurrent | Imatinib mesylate |
| | | and/or metastatic | |
| | | dermatofibrosarcoma | |
| | | protuberans | |
| | | Kit (CD117) positive | Imatinib mesylate |
| | | unsresectable and/or | |
| | | metastatic malignant | |
| | | gastrointestinal stromal | |
| | | tumors | |
| | | Resection of Kit | Imatinib mesylate |
| | | (CD117) positive GIST | |
| | | Carcinoma of the cervix | Topotecan hydrochloride |
| | | CD33 positive acute | Gemtuzumab ozogamicin |
| | | myeloid leukemia (60 yrs. | |
| | | or older) | |
| | | Rheumatoid arthritis | Rituximab |
| | | Urinary bladder, | BCG live |
| | | treatment & | |
| | | prophylaxis | |
| | | Acute promyelocytic | Arsenic tioxide |
| | | leukemia | |
| | | Colorectal carcinoma | Panitumumab |
| | | Mantle cell lymphoma | Bortezomib |
| | | Gastrointestinal stromal | Sunitinib malate |
| | | tumor | |
| | | Esophageal cancer | Profimer sodium |
| | | Endobronchial cancer | Profimer sodium |
| | | High-grade dysplasia in | Profimer sodium |
| | | Barrett's Esophagus | |
| | | Head & neck cancer | Docetaxel |
| | | Gastric adenocarcinoma | Docetaxel |
| | | Actinic keratosis | Imiquimod |
| | | Superficial basal cell | Imiquimod |
| | | carcinoma | |
| | | External genital warts | Imiquimod |
| | | Persistent and recurrent | Denileukin diftitox |
| | | cutaneous T-cell | |
| | | lymphoma | |
| | | Cutaneous lesions | Alitretinoin |
| | | (AIDS-related Kaposi's | |
| | | sarcoma) | |
| | | Cutaneous lesions | Bexarotene |
| | | (CTCL patients) | |
| | | Metastatic renal cell | Interleukin-2, human |
| | | carcinoma | recombinant |
| | | Metastatic melanoma | Interleukin-2, human |
| | | | recombinant |
| | | Multiple myeloma | Thalidomide |
| | | Palliative treatment of | Leuprolide acetate |
| | | advanced prostate | |
| | | cancer | |
| | | Cancer patients | Filgrastim (recombinant G-CSF) |
| | | receiving | |
| | | myelosuppressive | |
| | | therapy | |
| | | Patients with acute | Filgrastim (recombinant G |
| | | myeloid leukemia | |
| | | receiving induction or | |
| | | consolidation | |
| | | chemotherapy | |
| | | Cancer patients | Filgrastim (recombinant G |
| | | received bone marrow | |
| | | transplant | |
| | | Use Following | Sargramostim (recombinant |
| | | Induction | GM-CSF) |
| | | Chemotherapy in Acute | |
| | | Myelogenous Leukemia | |
| | | Non-Hodgkin's | Rituximab |
| | | Lymphoma | |
| | | Chronic lymphocytic | Rituximab |
| | | leukemia (CLL) | |
| | | Relapsed or refractory, | Ibritumomab tiuxetan |
| | | low-grade or follicular | |
| | | non-Hodgkin's | |
| | | lymphoma | |
| | | Previously untreated | Ibritumomab tiuxetan |
| | | follicular non- | |
| | | Hodgkin's lymphoma | |
| | | Patients with CD20 | Tositumomab |
| | | antigen-expressing | |
| | | relapsed or refractory, | |
| | | low grade, follicular, or | |
| | | transformed non- | |
| | | Hodgkin's lymphoma | |
| | | Chronic lymphocytic | Bendamustine hydrochloride |
| | | leukemia (CLL) | |
| | | Non-Hodgkin's | Bendamustine hydrochloride |
| | | lymphoma | |
| Cardiovascular | Cardiovascular | Vascular indications | Aspirin |
| Diseases | agents | (Ischemic Stroke, TIA, | |
| | | Acute MI, prevention of | |
| | | recurrent MI, unstable | |
| | | angina pectoris, chronic | |
| | | stable angina pectoris | |
| | | Revascularization | Aspirin |
| | | procedures (coronary | |
| | | artery bypass graft, | |
| | | percutaneous | |
| | | transluminal coronary | |
| | | angioplasty, carotid | |
| | | endarterectomy) | |
| | | Hypertension | Clonidine |
| | | Allergic reaction | Epinephrine |
| | | Heart failure | Carvedilol |
| | | Left ventricular | Carvedilol |
| | | dysfunction following | |
| | | myocardial infarction | |
| | | Nephropathy in Type 2 | Irbesartan |
| | | Diabetic patients | |
| | | Paroxysmal atrial | Propafenone hydrochloride |
| | | fibrillation/flutter | |
| | | (PAF) | |
| | | Paroxysmal | Propafenone hydrochloride |
| | | supraventricular | |
| | | tachycardia (PSVT) | |
| | | Ventricular tachycardia | Propafenone hydrochloride |
| | | Symptomatic atrial | Propafenone hydrochloride |
| | | fibrillation | |
| | | Mild to moderate heart | Digoxin |
| | | failure | |
| | | Blood pressure | Clevidipine butyrate |
| | | Primary hyperlipidemia | Colesevelam hydrochloride |
| | | Homozygous familial | Ezetimibe and simvastatin |
| | | hypercholesterolemia | |
| | | (HoFH) | |
| | | Homozygous | Ezetimibe |
| | | sitosterolemia | |
| | | Hyperlipidemia and | Fenofibrate |
| | | mixed dyslipidemia | |
| | | Hyperglyceridemia | Fenofibrate |
| | | Primary | Rosuvastatin calcium |
| | | dysbetalipoproteinemia | |
| | | Slowing of the | Rosuvastatin calcium |
| | | progression of | |
| | | atherosclerosis | |
| | | Very high triglycerides | Omega-3-acid ethyl esters |
| | | Angina pectoris | Nadolol |
| | | Edema | Furosemide |
| | | Acute ischemic stroke | Alteplase |
| | | Patent ductus arteriosus | Ibuprofen lysine |
| | | (PDA) in premature | |
| | | infances | |
| | | Myocardial infarction | Tenecteplase |
| Infectious | Anti-Infective | Fungal infection in | Amphotericin B |
| Diseases | Agents, Systemic | febrile, neutropenic | |
| | | patients | |
| | | Cryptococcal | Amphotericin B |
| | | Meningitis in HIV | |
| | | infected patients | |
| | | Aspergillus species, | Amphotericin B |
| | | Candida species and/or | |
| | | Cryptococcus species | |
| | | infection refractory to | |
| | | amphotericin B | |
| | | deoxycholate or in | |
| | | patients where renal | |
| | | impairment or | |
| | | unacceptable toxicity | |
| | | precludes the use of | |
| | | amphotericin B | |
| | | deoxycholate | |
| | | Visceral leishmaniasis | Amphotericin B |
| | | Pharyngitis/Tonsilitis | Clarithromycin |
| | | Acute maxillary | Clarithromycin |
| | | sinusitis | |
| | | Acute bacterial | Clarithromycin |
| | | exacerbation of chronic | |
| | | bronchitis | |
| | | Community-Acquired | Clarithromycin |
| | | pneumonia | |
| | | Uncomplicated skin and | Clarithromycin |
| | | skin structure infections | |
| | | H. pylori infections | Clarithromycin |
| | | Duodenal ulcer | Clarithromycin |
| | | Disseminated | Clarithromycin |
| | | mycobacterial | |
| | | infections | |
| | | Acute otitis media | Clarithromycin |
| | | Toxoplasmosis | Pyrimethamine |
| | | Acute Malaria | Pyrimethamine |
| | | Chemoprophylaxis of | Pyrimethamine |
| | | Malaria | |
| | | Ovarian cancer | Doxorubicin hydrochloride |
| | | AIDS-related kaposis | Doxorubicin hydrochloride |
| | | sarcoma | |
| | | Multiple myeloma | Doxorubicin hydrochloride |
| | | Hairy cell leukemia | Interferon alfa-2b, recombinant |
| | | Malignant Melanoma | Interferon alfa-2b, recombinant |
| | | Follicular Lymphoma | Interferon alfa-2b, recombinant |
| | | Condylomata | Interferon alfa-2b, recombinant |
| | | Acuminata | |
| | | Chronic Hepatitis C | Interferon alfa-2b, recombinant |
| | | Chronic Hepatitis B | Interferon alfa-2b, recombinant |
| | | Reducing the frequency | Interferon gamma-1 b |
| | | and severity of serious | |
| | | infections associated | |
| | | with Chronic | |
| | | Granulomatous Disease | |
| | | Pneumocystis carinii | Atovaquone |
| | | pneumonia in patients | |
| | | intolerant to | |
| | | trimethoprim- | |
| | | sulfamethoxazole | |
| | | (TMP-SMX) | |
| | | Mild-to-moderate PCP | Atovaquone |
| | | and intolerant to TMP- | |
| | | SMX | |
| | | Tuberculosis | Aminosalicylic acid |
| | | Treatment of CMV | Valganciclovir hydrochloride |
| | | retinitis in patients with | |
| | | AIDS | |
| | | Prevention of CMV | Valganciclovir hydrochloride |
| | | disease in kidney and | |
| | | heart transplant patients | |
| | | at high risk | |
| | | Cold sores | Valacyclovir hydrochloride |
| | | Genital Herpes (Prevent | Valacyclovir hydrochloride |
| | | transmission/treatment) | |
| | | Herpes Zoster | Valacyclovir hydrochloride |
| | | Chickenpox | Valacyclovir hydrochloride |
| | | CCR5-tropic HIV-1 | Maraviroc |
| | | who have evidence of | |
| | | viral replication and | |
| | | HIV-1 strains resistant | |
| | | to multiple | |
| | | antiretroviral agents | |
| | | HIV-1 infection | Raltegravir |
| | | HIV-1 infection | Efavirenz and emtricitabine and |
| | | | tenofovir |
| | | HIV-1 infection | Nevirapine |
| | | HIV-1 infection | Lamivudine and zidovudine |
| | | HIV-1 infection | Emtricitabine |
| | | HIV-1 infection | Zidovudine |
| | | Prevention of | Zidovudine |
| | | Maternal-Fetal HIV-1 | |
| | | Transmission | |
| | | HIV-1 infection | Abacavir Sulfate |
| | | HIV-1 infection | Emtricitabine and Tenofovir |
| | | | Disoproxil Fumarate |
| | | Neurocysticercosis | Albendazole |
| | | Hydatid Disease | Albendazole |
| | | Strongyloidiasis of the | Ivermectin |
| | | intestinal tract | |
| | | Onchocerciasis | Ivermectin |
| | | Cystic fibrosis patients | Tobramycin |
| | | with P. aeruginosa | |
| | | Complicated intra- | Ertapenem |
| | | abdominal infections | |
| | | Complicated skin and | Ertapenem |
| | | skin structure infection, | |
| | | including diabetic foot | |
| | | infections without | |
| | | osteomyelitis | |
| | | Community acquired | Ertapenem |
| | | pneumonia | |
| | | Complicated urinary | Ertapenem |
| | | tract infections | |
| | | including | |
| | | pyelonephritis | |
| | | Acute pelvic infections | Ertapenem |
| | | including postpartum | |
| | | endomyometritis, septic | |
| | | abortion and post | |
| | | surgical gynecologic | |
| | | infections | |
| | | Skin and skin structure | Meropenem |
| | | infections | |
| | | Intra-abdominal | Meropenem |
| | | infections | |
| | | Bacterial meningitis | Meropenem |
| | | Lower respiratory tract | Imipenem and Cilastatin |
| | | infection | |
| | | Intra-abdominal | Imipenem and Cilastatin |
| | | infections | |
| | | Gynecological | Imipenem and Cilastatin |
| | | infections | |
| | | Bacterial septicemia | Imipenem and Cilastatin |
| | | Bone and joint | Imipenem and Cilastatin |
| | | infections | |
| | | Endocarditis | Imipenem and Cilastatin |
| | | Polymicrobic infection | Imipenem and Cilastatin |
| | | Pharyngitis/Tonsilitis | Cefuroxime axetil |
| | | Acute Bacterial Otitis | Cefuroxime axetil |
| | | Media | |
| | | Acute bacterial | Cefuroxime axetil |
| | | maxillary sinusitis | |
| | | Acute bacterial | Cefuroxime axetil |
| | | exacerbations of | |
| | | chronic bronchitis and | |
| | | secondary bacterial | |
| | | infections of acute | |
| | | bronchitis | |
| | | Uncomplicated urinary | Cefuroxime axetil |
| | | tract infections | |
| | | Uncomplicated | Cefuroxime axetil |
| | | gonorrhea | |
| | | Early Lyme's disease | Cefuroxime axetil |
| | | (erythema migrans) | |
| | | Impetigo | Cefuroxime axetil |
| | | Bacterial Septicemia | Ceftazidime |
| | | Bone and joint | Ceftazidime |
| | | infections | |
| | | Gynecologic infections | Ceftazidime |
| | | Central nervous system | Ceftazidime |
| | | infections | |
| | | Septicemia | Cefuroxime |
| | | Meningitis | Cefuroxime |
| | | Gonorrhea | Cefuroxime |
| | | Bone and joint infection | Cefuroxime |
| | | Surgical prophylaxis | Ceftriaxone sodium |
| | | Upper Respiratory tract | Erythromycin ethylsuccinate |
| | | infections | |
| | | Listeriosis | Erythromycin ethylsuccinate |
| | | Pertussis | Erythromycin ethylsuccinate |
| | | Skin and skin structure | Erythromycin ethylsuccinate |
| | | infections | |
| | | Diphtheria | Erythromycin ethylsuccinate |
| | | Erythrasma | Erythromycin ethylsuccinate |
| | | Intestinal amebiasis | Erythromycin ethylsuccinate |
| | | Acute pelvic | Erythromycin ethylsuccinate |
| | | inflammatory disease | |
| | | Syphilis | Erythromycin ethylsuccinate |
| | | Conjunctivitis of | Erythromycin ethylsuccinate |
| | | newborns | |
| | | Pneumonia of infancy | Erythromycin ethylsuccinate |
| | | Urogenital infections | Erythromycin ethylsuccinate |
| | | during pregnancy | |
| | | Legionnaires' disease | Erythromycin ethylsuccinate |
| | | Nongonococeal | Erythromycin ethylsuccinate |
| | | urethritis | |
| | | Initial and recurrent | Erythromycin ethylsuccinate |
| | | attacks of Rheumatic | |
| | | fever | |
| | | Staphylococcus aureus | Daptomycin |
| | | bloodstream infections | |
| | | (bacteremia), including | |
| | | with right-sided | |
| | | infective endocarditis | |
| | | Community acquired | Telithromycin |
| | | pneumonia | |
| | | Cystic fibrosis | Bismuth subcitrate potassium, |
| | | | metronidazole, and tetracycline |
| | | | hydrochloride |
| | | Chronic pancreatitis | Bismuth subcitrate potassium, |
| | | | metronidazole, and tetracycline |
| | | | hydrochloride |
| | | Obstruction (pancreatic | Bismuth subcitrate potassium, |
| | | and biliary duct | metronidazole, and tetracycline |
| | | lithiasis, pancreatic and | hydrochloride |
| | | duodenal neoplasms, | |
| | | ductal stenosis) | |
| | | Other pancreatic | Bismuth subcitrate potassium, |
| | | disease (hereditary, post | metronidazole, and tetracycline |
| | | traumatic and allograft | hydrochloride |
| | | pancreatitis, | |
| | | hemochromatosis, | |
| | | Shwachman's | |
| | | Syndrome, lipomatosis, | |
| | | hyperparathyroidism) | |
| | | Poor mixing (Billroth II | Bismuth subcitrate potassium, |
| | | gastrectomy, other | metronidazole, and tetracycline |
| | | types of gastric bypass | hydrochloride |
| | | surgery, gastrinoma) | |
| | | Active pulmonary and | Cycloserine |
| | | extrapulmonary | |
| | | tuberculosis | |
| | | Urinary tract infections | Cycloserine |
| | | Vancomycin-resistant | Linezolid |
| | | Enterococcus faecium | |
| | | infections | |
| | | Nosocomial pneumonia | Linezolid |
| | | Uncomplicated skin and | Linezolid |
| | | skin structure infections | |
| | | Ear, nose and throat | Amoxicillin |
| | | infections | |
| | | Genitourinary tract | Amoxicillin |
| | | infections | |
| | | Gonorrhea | Amoxicillin |
| | | Scarlet fever | Penicillin G benzathine and |
| | | | penicillin G procaine |
| | | Erysipelas | Penicillin G benzathine and |
| | | | penicillin G procaine |
| | | Venereal infections | Penicillin G benzathine |
| | | Chorea | Penicillin G benzathine |
| | | Intra-abdominal | Ticarcillin disodium and |
| | | infections | clavulanate potassium |
| | | Appendicitis | Piperacillin and tazobactum |
| | | Postpardum | Piperacillin and tazobactum |
| | | endometritis | |
| | | Pelvic inflammatory | Piperacillin and tazobactum |
| | | disease | |
| | | Acute bacterial sinusitis | Moxifloxacin hydrochloride |
| | | Acute bacterial | Moxifloxacin hydrochloride |
| | | Exacerbation of | |
| | | Chronic Bronchitis | |
| | | Complicated intra- | Moxifloxacin hydrochloride |
| | | abdominal infections | |
| | | Chronic bacterial | Ciprofloxacin |
| | | Prostatitis | |
| | | Febrile neutropenic | Ciprofloxacin |
| | | Inhalation anthrax | Ciprofloxacin |
| | | Acute uncomplicated | Ciprofloxacin hydrochloride |
| | | Cystitis in females | |
| | | Chronic bacterial | Ciprofloxacin hydrochloride |
| | | prostatitis | |
| | | Infectious diarrhea | Ciprofloxacin hydrochloride |
| | | Typhoid fever | Ciprofloxacin hydrochloride |
| | | Uncomplicated cervical | Ciprofloxacin hydrochloride |
| | | and urethral gonorrhea | |
| | | Pyelonephritis | Ciprofloxacin hydrochloride |
| | | Helicobacter pylori | Bismuth subcitrate potassium, |
| | | infection | metronidazole and tetracycline |
| | | | hydrochloride |
| | | Duodenal ulcer | Bismuth subcitrate potassium, |
| | | | metronidazole and tetracycline |
| | | | hydrochloride |
| | | Peritonitis | Caspofungin acetate |
| | | Intra-abdominal | Caspofungin acetate |
| | | abscesses | |
| | | Pleural space infections | Caspofungin acetate |
| | | Esophageal candidiasis | Caspofungin acetate |
| | | Invasive aspergillosis | Caspofungin acetate |
| | | Prophylaxis of Candida | Micafungin sodium |
| | | Infections in patients | |
| | | undergoing | |
| | | hematopietic stem cell | |
| | | transplantation | |
| | | Esophageal candidiasis | Micafungin sodium |
| | | Candidemia, acute | Micafungin sodium |
| | | disseminated | |
| | | candidiasis, candida | |
| | | peritonitis and | |
| | | abscesses | |
| | | Acute, uncomplicated | Artemether and lumefantrine |
| | | malaria | |
| | | Toxoplasmosis | Pyrimethamine |
| | | Chemoprophylaxis of | Pyrimethamine |
| | | malaria | |
| | | Chronic hepatitis C | Peginterferon alfa 2b |
| | | virus infection in | |
| | | patients with | |
| | | compensated liver | |
| | | disease | |
| | | Malaria | Atovaquene and proguanil |
| | | | hydrochloride |
| | | External condylomata | Interferon alfa-n3 |
| | | acuminata (refractory/ | |
| | | recurring) | |
| | | Chronic Hepatitis B | Lamivudine |
| | | Influenza (also | Zanamivir |
| | | prophylaxis) | |
| | | RSV disease | Palivizumab |
| | | Cytomegalovirus | Valganciclovir hydrochloride |
| | | (CMV) Retinitis | |
| | | (treatment/prevention) | |
| | | Herpes Zoster | Acyclovir |
| | | infections | |
| | | Chickenpox | Acyclovir |
| | | Dermatitis | Dapsone |
| | | herpetiformis | |
| | | Leprosy | Dapsone |
| | | Elevated urinary pH | Potassium acid phosphate |
| | | Traveler's Diarrhea | Rifaximin |
| | | Non-Hodgkin's | Rituximab |
| | | Lymphoma | |
| | | Cutaneous lesions in | Bexarotene |
| | | patients with CTCL | |
| | | (Stage IA and IB) with | |
| | | refractory or persistent | |
| | | disease after other | |
| | | therapy (topical) | |
| | | Acute promyelocytic | Arsenic trioxide |
| | | leukemia | |
| | | Mantle cell lymphoma | Bortexomib |
| | | Esophageal cancer | Porfimer sodium |
| | | Endobronchial Cancer | Porfimer sodium |
| | | High-Grade dysplasia | Porfimer sodium |
| | | in Barrett's Esophagus | |
| | | Hormone refractory | Docetaxel |
| | | prostate cancer | |
| | | Gastric adenocarcinoma | Docetaxel |
| | | (GC) | |
| | | Squamous cell | Docetaxel |
| | | carcinoma of the head | |
| | | and neck cancer | |
| | | Eradication of nasal | Mupirocin calcium |
| | | colonization with | |
| | | methicillin-resistant S. aureus | |
| | | Bacterial conjunctivitis | Azithromycin |
| | | Corneal ulcer | Levofloxacin |
| | | Acute otitis externa | Ciprofloxacin and |
| | | | dexamethasone |
| | Anti-infectives | Impetigo | Retapamulin |
| | | Ears, nose and throat | Amoxicillin |
| | | Genitourinary tract | Amoxicillin |
| | | Skin and skin structure | Amoxicillin |
| | | Lower respiratory tract | Amoxicillin |
| | | Gonorrhea | Amoxicillin |
| | | (uncomplicated) | |
| | | H. pylori infections | Amoxicillin |
| | | Duodenal ulcer disease | Amoxicillin |
| | | Otitis media | Amoxicillin |
| | | Urinary tract | Amoxicillin |
| | | Sinusitis | Amoxicillin |
| | | Acute bacterial sinusitis | Moxifloxacin hydrochloride |
| | | Acute bacterial | Moxifloxacin hydrochloride |
| | | exacerbation of chronic | |
| | | bronchitis | |
| | | Community acquired | Moxifloxacin hydrochloride |
| | | pneumonia | |
| | | Complicated intra- | Moxifloxacin hydrochloride |
| | | abdominal infections | |
| | | Secondary infected | Mupirocin calcium cream |
| | | traumatic skin lesions | |
| | | Nasal colonization with | Mupirocin calcium ointment |
| | | methicillin-resistant S. aureus | |
| | | in adults and | |
| | | health workers | |
| | | Acne vulgaris | Clindamycin and benzoyl |
| | | | peroxide |
| | | Pharyngitis/tonsillitis | Clarithromycin |
| | | Disseminated | Clarithromycin |
| | | mycobacterial | |
| | | infections | |
| | | Duodenal ulcer | Clarithromycin |
| | | Early lyme disease | Cefuroxime axetil |
| | | Nosocomial Pneumonia | Ciprofloxacin |
| | | Cystitis in females | Ciprofloxacin hydrochloride |
| | | Bone and joint | Ciprofloxacin hydrochloride |
| | | Infectious diarrhea | Ciprofloxacin hydrochloride |
| | | Typhoid fever | Ciprofloxacin hydrochloride |
| | | Pyelonephritis | Ciprofloxacin hydrochloride |
| | | Blood infections | Daptomycin |
| | | Listeriosis | Erythromycin ethylsuccinate |
| | | Pertussis | Erythromycin ethylsuccinate |
| | | Diphtheria | Erythromycin ethylsuccinate |
| | | Erythrasma | Erythromycin ethylsuccinate |
| | | Intestinal amebiasis | Erythromycin ethylsuccinate |
| | | Pelvic inflammatory | Erythromycin ethylsuccinate |
| | | disease | |
| | | Syphilis | Erythromycin ethylsuccinate |
| | | Conjunctivitis of | Erythromycin ethylsuccinate |
| | | newborns | |
| | | Pneumonia of infancy | Erythromycin ethylsuccinate |
| | | Urogenital infections | Erythromycin ethylsuccinate |
| | | Nongonococeal | Erythromycin ethylsuccinate |
| | | urethritis | |
| | | Legionnaires' | Erythromycin ethylsuccinate |
| | | Rheumatic fever | Erythromycin ethylsuccinate |
| | | Bacterial septicemia | Ceftazidime |
| | | Gynecologic infections | Ceftazidime |
| | | Central nervous system | Ceftazidime |
| | | infections | |
| | | Inhalation anthrax | Levofloxacin |
| | | Chronic bacterial | Levofloxacin |
| | | prostatitis | |
| | | Acute pyelonephritis | Levofloxacin |
| | | Bacterial meningitis | Meropenem |
| | | Endocarditis | Imipenem and cilastatin |
| | | Polymicrobic infections | Imipenem and cilastatin |
| | | Acne rosacea | Sodium sulfacetamide |
| | | Seborrheic dermatitis | Sodium sulfacetamide |
| | | Vancomycin-resistant | Linezolid |
| | | Enterococcus faecium | |
| | | infections | |
| | | Athlete's foot | Undecylenic acid and |
| | | | chloroxylenol |
| | | Ringworm | Undecylenic acid and |
| | | | chloroxylenol |
| | | Symptomatic | Clotrimazole and betamethasone |
| | | inflammatory tinea | dipropionate |
| | | pedis | |
| | | Symptomatic | Clotrimazole and betamethasone |
| | | inflammatory tinea | dipropionate |
| | | cruris | |
| | | Symptomatic | Clotrimazole and betamethasone |
| | | inflammatory tinea | dipropionate |
| | | corporis | |
| | | Cutaneous mycotic | Nystatin |
| | | infections | |
| | | Mucocutaneous | Nystatin |
| | | mycotic infections | |
| | | Diaper dermatitis | Miconazole nitrate |
| | | Cold sores | Penciclovir |
| | | Localized infections | Sodium oxychlorosene |
| | | Leprosy | Dapsone |
| | | 2^{nd} and 3^{rd} degree burns | Mafenide acetate |
| | | Head lice | Benzyl alcohol |
| | Antibacterials | Ear, nose and throat | Amoxicillin |
| | | infections | |
| | | Genitourinary tract | Amoxicillin |
| | | infections | |
| | | Gonorrhea | Amoxicillin |
| | | Skin and skin structure | Amoxicillin |
| | | Lower respiratory tract | Amoxicillin |
| | | Gonorrhea | Amoxicillin |
| | | h. pylori | Amoxicillin |
| | | Duodenal ulcer | Amoxicillin |
| | | Otitis Media | Amoxicillin and clavulanate |
| | | | potassium |
| | | Sinusitis | Amoxicillin and clavulanate |
| | | | potassium |
| | | Urinary tract infection | Amoxicillin and clavulanate |
| | | | potassium |
| | | Pharyngitis/tonsillitis | Cefuroxime acetil |
| | | Exacerbations of | Cefuroxime acetil |
| | | chronic bronchitis and | |
| | | secondary bacterial | |
| | | infections of acute | |
| | | bronchitis | |
| | | Early lyme disease | Cefuroxime acetil |
| | | Nosocomial pneumonia | Ciprofloxacin |
| | | Bone and joint | Ciprofloxacin |
| | | infections | |
| | | Intra-abdominal | Ciprofloxacin |
| | | infections | |
| | | Chronic bacterial | Ciprofloxacin |
| | | prostatitis | |
| | | Emprical therapy for | Ciprofloxacin |
| | | febril neutropenic | |
| | | patients | |
| | | Pyelonephritis | Ciprofloxacin |
| | | Cystis in females | Ciprofloxacin hydrochloride |
| | | Infectious diarrhea | Ciprofloxacin hydrochloride |
| | | Typhoid fever | Ciprofloxacin hydrochloride |
| | | Listeriosis | Erythromycin ethylsuccinate |
| | | Pertussis | Erythromycin ethylsuccinate |
| | | Diphtheria | Erythromycin ethylsuccinate |
| | | Erythrasma | Erythromycin ethylsuccinate |
| | | Intestinal amebiasis | Erythromycin ethylsuccinate |
| | | Pelvic inflammatory | Erythromycin ethylsuccinate |
| | | disease | |
| | | Syphilis | Erythromycin ethylsuccinate |
| | | Conjunctivitis of | Erythromycin ethylsuccinate |
| | | newborns | |
| | | Pneumonia of infancy | Erythromycin ethylsuccinate |
| | | Urogenital infections | Erythromycin ethylsuccinate |
| | | during pregnancy | |
| | | Bacterial septicemia | Ceftazidime |
| | | Gynecological | Ceftazidime |
| | | infections | |
| | | Central nervous system | Ceftazidime |
| | | infections | |
| | | Endocarditis | Imipenem and cilastatin |
| | | Polymicrobic infections | Imipenem and cilastatin |
| | | Meningitis | Ceftriaxone sodium |
| | | Tuberculosis, | Cycloserine |
| | | pulmonary and | |
| | | extrapulmonary | |
| Ophthalmic | Ophthalmic | Legionnaires' disease | Erythromycin ethylsuccinate |
| Diseases | preparations | Neovascular (wet) age- | Ranibizumab |
| | | related macular | |
| | | degeneration | |
| | | Subfoveal choroidal | Verteporfin |
| | | neovascularization due | |
| | | to age-related macular | |
| | | degeneration, | |
| | | pathologic myopia or | |
| | | presumed ocular | |
| | | histoplasmosis | |
| | Antihistamine & | Prevention of itching | Epinastine hydrochloride |
| | Mast Cell | associated with allergic | |
| | Stabilizer | conjunctivitis | |
| | Combinations | Ocular itching | Olopatadine hydrochloride |
| | | associated with allergic | |
| | | conjunctivitis | |
| | Antihistamines & | Itchy eyes | Ketotifen |
| | Combinations | | |
| | Anti-Infectives | Bacterial conjunctivitis | Azithromycin |
| | | Bacterial conjunctivitis | Besifloxacin |
| | | Corneal ulcer caused by | Levofloxacin |
| | | bacteria | |
| | | Bacterial conjunctivitis | Levofloxacin |
| | | Bacterial conjunctivitis | Moxifloxacin hydrochloride |
| | Artificial | Increased tear | Cyclosporine |
| | Tears/Lubricants | production | |
| | & Combinations | | |
| | Beta Adrenergic | Elevated intraocular | Timolol |
| | Blocking Agents | pressure in patients | |
| | & Combinations | with ocular | |
| | | hypertension or open- | |
| | | angle glaucoma | |
| | | Reduction of elevated | Brimonidine tartrate and |
| | | intraocular pressure in | Timolol maleate |
| | | patients with glaucoma | |
| | | or ocular hypertension | |
| | Carbonic | Elevated intraocular | Dorzolamide hydrochloride |
| | Anhydrase | pressure in patients | |
| | Inhibitors & | with ocular | |
| | Combinations | hypertension or open- | |
| | | angle glaucoma | |
| | Mast Cell | Prevention of itching of | Pemirolast potassium |
| | Stabilizers | the eye due to allergic | |
| | | conjunctivitis | |
| | Prostaglandins | Reduction of elevated | Bimatoprost |
| | | intraocular pressure in | |
| | | patients with open | |
| | | angle glaucoma or | |
| | | ocular hypertension | |
| | | Reduction of elevated | Travoprost |
| | | intraocular pressure in | |
| | | patients with open | |
| | | angle glaucoma or | |
| | | ocular hypertension | |
| | Sympathomimetics | Reduction of elevated | Brimonidine tartrate |
| | & Combinations | intraocular pressure in | |
| | | patients with open | |
| | | angle glaucoma or | |
| | | ocular hypertension | |
| Diabetes | | Improve glycemic | Insulin glulisine |
| | | control in adults and | |
| | | children with diabetes | |
| | | mellitus | |
| | | Control of | Insulin lispro |
| | | hyperglycemia in | |
| | | patients with diabetes | |
| | | mellitus | |
| | | Insulin resistance | Insulin, human |
| | | Improve glycemic | Insulin glargine |
| | | control in adults and | |
| | | children with type 1 | |
| | | diabetes mellitus and in | |
| | | adults with type 2 | |
| | | diabetes mellitus | |
| | | Severe hypoglycemic | Glucagon |
| | | (low blood sugar) | |
| | | reactions in patients | |
| | | with diabetes | |
| | | Type 2 Diabetes | Exenatide |
| | | Mellitus | |
| | | Diabetes mellitus | Glucagon-like peptide-1 |
| | | Diabetes mellitus | Fibronectin, recombinant |
| | | Diabetes insipidus | Vasopressin |
| Other Diseases | | Postoperative | Vasopressin |
| | | abdominal distention | |
| | | Delaying time to | Interferon gamma-1b |
| | | disease progression in | |
| | | patients with severe, | |
| | | malignant osteopetrosis | |
| | | Anemia | Epoetin alfa |
| | | Anemia | Darbepoetin alfa |
| | | Anemia | Methoxy polyethylene glycol- |
| | | | epoetin beta |
| | | Endometriosis | Leuprolide acetate |
| | | Uterine leiomyomata | Leuprolide acetate |
| | | (Fibroids) | |
| | | Patients with severe | Filgrastim (recombinant G-CSF) |
| | | chronic neutropenia | |
| | | Patients Undergoing | Filgrastim (recombinant G |
| | | Peripheral Blood | |
| | | Progenitor Cell | |
| | | Collection and Therapy | |
| | | Use in Mobilization and | Sargramostim (recombinant |
| | | Following | GM-CSF) |
| | | Transplantation of | |
| | | Autologous Peripheral | |
| | | Blood Progenitor Cells | |
| | | Use in Myeloid | Sargramostim (recombinant |
| | | Reconstitution After | GM-CSF) |
| | | Autologous Bone | |
| | | Marrow | |
| | | Transplantation | |
| | | Use in Myeloid | Sargramostim (recombinant |
| | | Reconstitution After | GM-CSF) |
| | | Allogeneic Bone | |
| | | Marrow | |
| | | Transplantation | |
| | | Use in Bone Marrow | Sargramostim (recombinant |
| | | Transplantation Failure | GM-CSF) |
| | | or Engraftment Delay | |
| | | Pediatric patients who | Somatrem |
| | | have growth failure due | |
| | | to a lack of adequate | |
| | | endogenous growth | |
| | | hormone secretion | |
| | | Pediatric patients who | Somatropin |
| | | have growth failure due | |
| | | to a lack of adequate | |
| | | endogenous growth | |
| | | hormone secretion | |
| | | Growth hormone | Somatropin |
| | | deficiency | |
| | | Short stature associated | Somatropin |
| | | with Turner Syndrome | |
| | | in pediatric patients | |
| | | Use in Myeloid | Sargramostim (recombinant |
| | | Reconstitution After | GM-CSF) |
| | | Allogeneic Bone | |
| | | Marrow | |
| | | Transplantation | |

In addition to the exemplary APIs provided in Table 2, other suitable APIs include, but are not limited to, antibodies, antibody fragments (e.g., mini-antibodies, Fab, and antigenic binding domain rabbit antibody IgG), adnectins, insulin, interleukins, colony stimulating factors, hormones (e.g., growth hormone, vasopressin, luteinizing hormone-releasing hormone), erythropoietin, interferons, aptamers (e.g., PEGylated aptamers), siRNA, antisense RNA, nucleotides (e.g., modified nucleotides), PEGylated proteins, enzymes, blood clotting factors, cytokines, growth factors, vaccine agents (e.g., microorganisms or components thereof, toxoids), small molecules, and combinations thereof.

The API can be natural, synthetic, or partially synthetic products. In one embodiment, the API is a recombinant protein. In another embodiment, the API is a synthetic oligonucleotide. Nucleic acids used with embodiments of the invention can include various types of nucleic acids that can function to provide a therapeutic effect. Exemplary types of nucleic acids can include, but are not limited to, ribonucleic acids (RNA), deoxyribonucleic acids (DNA), small interfering RNA (siRNA), micro RNA (miRNA), piwi-interacting RNA (piRNA), short hairpin RNA (shRNA), antisense nucleic acids, aptamers, ribozymes, locked nucleic acids and catalytic DNA.

The API can be present in any suitable and appropriate amount of the formulation, provided upon administration of the formulation, a safe and therapeutically effective amount of API is delivered. In specific embodiments, the API can be present in about 0.01 wt. % to about 50 wt. % of the formulation, in about 0.1 wt. % to about 30 wt. % of the formulation, or in about 1 wt. % to about 10 wt. % of the formulation.

### Carriers

Carriers can be administered in combination with the API to improve delivery of the APIs to the targeted tissues and cells. Carriers can also protect the API from damage or premature degradation.

In various embodiments a nucleic acid can be used as an API, and a carrier can be attached to the nucleic acid to form nucleic acid complexes. Carrier agents used with embodiments of the invention can include those compounds that can be complexed with nucleic acids in order to preserve the activity of the nucleic acids during the manufacturing and delivery processes. Typically, nucleic acid/carrier complexes self-assemble when brought into contact with one another, for example, in an aqueous solution. For example, a complex may form due to the charge-charge interactions between a negatively charged nucleic acid and a positively charged carrier agent. In some instances, particles (e.g., micelles, lipoplexes or liposomes) can be formed when the API interacts with a carrier agent. Exemplary classes of suitable carrier agents can include cationic compounds (compounds having a net positive charge) and charge neutral compounds.

Carrier agents can include cationic polymers that are capable of efficiently condensing the API into nanoparticles, termed “polyplexes,” by self-assembly via electrostatic interactions. Sometimes the cationic polymer includes one or more functional groups that can be modified with ligands, such as cell-targeting molecules. Examples of cationic polymers include, but are not limited to polycations containing cyclodextrin, histones, cationized human serum albumin, aminopolysaccharides such as chitosan, peptides such as poly-L-lysine, poly-L-ornithine, and poly(4-hydroxy-L-proline ester, and polyamines such as polyethylenimine (PEI), polypropylenimine, polyamidoamine dendrimers, and poly(beta-aminoesters).

Another class of carrier includes cationic lipids. Cationic lipid carriers are commercially available and include, but are not limited to 1,2-dioleoyl-3-trimethylammonium propane (DOTAP), N-methyl-4-(dioleyl)methylpyridinium (SAINT-2), 3β-[N-(N′,N′-dimethylaminoethane)-carbamoyl]cholesterol (DC-Chol), or a gemini surfactant (e.g., a surfactant having two conventional surfactant molecules chemically bonded together by a spacer), such as GS1 (a sugar-based gemini surfactant), as well as the neutral lipid dioleoylphosphatidylethanolamine (DOPE) and cholesterol. Addition of polyanionic nucleic acids to mixtures of cationic lipids or liposomes results in the self-assembly of particles termed “lipoplexes.” Other carrier agents can include solid nucleic acid lipid nanoparticles (SNALPs), liposomes and the like. In one embodiment, the carrier can be conjugated to one or more molecules that target specific cell types. Examples of targeting agents include antibodies and peptides which recognize and bind to specific cell surface molecules.

In some embodiments, carriers used with embodiments of the invention can include peptides that facilitate delivery of an API to a cell of interest. For example, exemplary peptides can associate with a nucleic acid and facilitate delivery of that nucleic acid to the cytoplasm of a cell. As used herein, the term “peptide” shall include any compound containing two or more amino-acid residues joined by amide bond(s) formed from the carboxyl group of one amino acid (residue) and the amino group of the next one. As such, peptides can include oligopeptides, polypeptides, proteins, and the like.

In some embodiments, carrier used with embodiments of the invention can include peptides that have at least two domains, such as a cellular penetration domain and a nucleic acid binding domain. As used herein, the term “cellular penetration domain” shall refer to a region of a peptide molecule that functions to facilitate entry of the molecule into a cell. As used herein, the term “nucleic acid binding domain” shall refer to a region of a peptide molecule that functions to bind with nucleic acids.

It will be appreciated that many different peptides for targeted delivery of the API (e.g., a nucleic acid) are contemplated herein. One exemplary peptide, known as MPG, is a 27 amino acid bipartite amphipathic peptide composed of a hydrophobic domain derived from HIV-1 gp41 protein and a basic domain from the nuclear localization sequence (NLS) of SV40 large T antigen (GALFLGFLGAAGSTMGAWSQPKKKRKV) (commercially available as the N-TER Nanoparticle siRNA Transfection System from Sigma-Aldrich, St. Louis, Mo.). Another exemplary peptide, known as MPGΔ^{NLS}, is also a 27 amino acid bipartite amphipathic peptide (GALFLGFLGAAGSTMGAWSQPKSKRKV). Other exemplary peptides can include poly-arginine fusion peptides. Still other exemplary peptides include those with protein transduction domains linked with a double-stranded RNA binding domain, such as PTD-DRBD protein.

### Diseases

The formulations described herein can be used to treat a variety of conditions by using a suitable API in the injectable formulation. Such conditions or diseases include, but are not limited to, retinal diseases, front of the eye diseases, inflammatory diseases, autoimmune diseases, rheumatic diseases, cancers, cardiology disorders, neurological disorders, clotting disorders, anemia arising from cancer chemotherapy, transplant rejection, infections, and pain, or a combination thereof. Certain specific conditions that can be treated using a formulation described herein include age-related macular degeneration (wet and dry), diabetic macular edema (DME), glaucoma, keratoconjunctivitis sicca (KCS) or dry eye syndrome, multiple sclerosis, rheumatoid arthritis, Alzheimer's disease, Parkinson's disease, amyotrophic lateral sclerosis (ALS), hepatitis B and C, and systemic lupus erythematosus, or a combination thereof. Additional suitable diseases, disorders and/or conditions are described, for example, in Table 2 above. Additional suitable diseases, disorders and/or conditions can be found, for example, at the Physician's Desk Reference 64^{th} Edition (2010).

The following Embodiments are intended to illustrate the above invention and should not be construed as to narrow its scope.

### Embodiments

[1] The present invention provides a formulation comprising:

(a) a biocompatible solvent system;

(b) a biodegradable polymer that is substantially soluble in the biocompatible solvent system, the biodegradable polymer comprising a polysaccharide comprising a unit of formula (I):

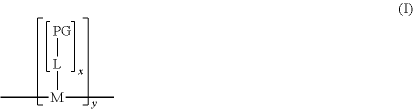

wherein,

each M is independently a monosaccharide unit;

each L is independently a suitable linking group or a direct bond;

each PG is independently a pendent group;

each x is independently 0 to about 3, such that when x is 0, the bond between L and M is absent;

y is 3 to about 10,000; and

(c) an active pharmaceutical ingredient (API) that is substantially insoluble in the biocompatible solvent system.
[2] The present invention also provides the formulation of embodiment [1], wherein the solvent system is water immiscible.
[3] The present invention also provides the formulation of any one of embodiments [1]-[2], wherein the solvent system is present in about 10 wt. % to about 40 wt. % of the formulation.
[4] The present invention also provides the formulation of any one of embodiments [1]-[2], wherein the solvent system is present in about 40 wt. % to about 90 wt. % of the formulation.
[5] The present invention also provides the formulation of any one of embodiments [1]-[4], wherein the solvent system comprises two or more organic solvents.
[6] The present invention also provides the formulation of any one of embodiments [1]-[4], wherein the solvent system comprises at least one organic solvent that is miscible to dispersible in aqueous medium or body fluid, at least one organic solvent that is immiscible to insoluble in aqueous medium or body fluid, or a combination thereof.
[7] The present invention also provides the formulation of any one of embodiments [1]-[4], wherein the solvent system comprises a combination of: (a) at least one organic solvent that is miscible to dispersible in aqueous medium or body fluid, and (b) at least one organic solvent that is immiscible to insoluble in aqueous medium or body fluid.
[8] The present invention also provides the formulation of any one of embodiments [1]-[4], wherein the solvent system comprises a combination of: (a) at least one organic solvent that is miscible to dispersible in aqueous medium or body fluid, and (b) at least one organic solvent that is immiscible to insoluble in aqueous medium or body fluid; and the polymer has greater solubility in the miscible to dispersible solvent, as compared to the immiscible to insoluble solvent.
[9] The present invention also provides the formulation of any one of embodiments [1]-[4], wherein the solvent system comprises ethyl heptanoate, glycofural, benzyl benzoate, glycerol tributyrate, dimethylisosorbide, or a mixture thereof.
[10] The present invention also provides the formulation of any one of embodiments [1]-[9], wherein the solvent system is liquid at ambient and physiological temperature.
[11] The present invention also provides the formulation of any one of embodiments [1]-[10], wherein the solvent system comprises at least one aprotic solvent.
[12] The present invention also provides the formulation of any one of embodiments [1]-[11], wherein the solvent system has a solubility range of miscible to dispersible in aqueous medium or bodily fluids.
[13] The present invention also provides the formulation of any one of embodiments [1]-[11], wherein the solvent system has a solubility range of immiscible to non-dispersible in aqueous medium or bodily fluids.
[14] The present invention also provides the formulation of any one of embodiments [1]-[13], wherein the solvent system is capable of dissipation, diffusion, absorption, degradation, or a combination thereof, into body fluid upon placement within a body tissue.
[15] The present invention also provides the formulation of any one of embodiments [1]-[14], wherein the solvent system is non-aqueous.
[16] The present invention also provides the formulation of any one of embodiments [1]-[15], wherein the solvent system comprises at least one biodegradable organic solvent.
[17] The present invention also provides the formulation of any one of embodiments [1]-[16], wherein the solvent system comprises at least one biodegradable organic solvent, present in about 10 wt. % to about 40 wt. % of the formulation.
[18] The present invention also provides the formulation of any one of embodiments [1]-[16], wherein the biodegradable polymer is present in about 40 wt. % to about 90 wt. % of the formulation.
[19] The present invention also provides the formulation of any one of embodiments [1]-[18], wherein the biodegradable polymer has a solubility of at least about 50 g/L in the biocompatible solvent system, at 25° C. and 1 atm.
[20] The present invention also provides the formulation of any one of embodiments [1]-[19], wherein the biodegradable polymer has a viscosity of less than about 5,000 cP at 37° C.
[21] The present invention also provides the formulation of any one of embodiments [1]-[20], wherein the biodegradable polymer is hydrophobic.
[22] The present invention also provides the formulation of any one of embodiments [1]-[21], wherein the monosaccharide units comprise glucopyranose units.
[23] The present invention also provides the formulation of any one of embodiments [1]-[21], wherein the monosaccharide units comprise D-glucopyranose.
[24] The present invention also provides the formulation of any one of embodiments [1]-[21], wherein the monosaccharide units comprise α-D-glucopyranose.
[25] The present invention also provides the formulation of any one of embodiments [1]-[21], wherein the monosaccharide units comprise glucopyranose units linked by α(1→4) glycosidic bonds.
[26] The present invention also provides the formulation of any one of embodiments [1]-[21], wherein the monosaccharide units comprise glucopyranose units linked by α(1→6) glycosidic bonds.
[27] The present invention also provides the formulation of any one of embodiments [1]-[21], wherein the monosaccharide units comprise non-macrocyclic poly-α(1→4) glucopyranose.
[28] The present invention also provides the formulation of any one of embodiments [1]-[21], wherein the monosaccharide units comprise non-macrocyclic poly-α(1→6) glucopyranose.
[29] The present invention also provides the formulation of any one of embodiments [1]-[21], wherein the monosaccharide units comprise glucopyranose units and at least about 90% of the glucopyranose units are linked by α(1→4) glycosidic bonds.
[30] The present invention also provides the formulation of any one of embodiments [1]-[21], wherein at least about 90% of the monosaccharides in the polysaccharide are the same type.
[31] The present invention also provides the formulation of any one of embodiments [1]-[21], wherein the polysaccharide is a homopolysaccharide.
[32] The present invention also provides the formulation of any one of embodiments [1]-[21], wherein the polysaccharide is a heteropolysaccharide.
[33] The present invention also provides the formulation of any one of embodiments [1]-[32], wherein the polysaccharide has a glass transition temperature (Tg) of at least about 35° C. (about 40° C. to about 150° C.).
[34] The present invention also provides the formulation of any one of embodiments [1]-[32], wherein the polysaccharide has a glass transition temperature (Tg) of −30° C. to about 0° C.
[35] The present invention also provides the formulation of any one of embodiments [1]-[34], wherein the polysaccharide comprises up to about 5,000 glucopyranose units.
[36] The present invention also provides the formulation of any one of embodiments [1]-[35], wherein the polysaccharide has an average MW up to about 1,000,000 Da.
[37] The present invention also provides the formulation of any one of embodiments [1]-[35], wherein the polysaccharide has an average MW up to about 300,000 Da.
[38] The present invention also provides the formulation of any one of embodiments [1]-[35], wherein the polysaccharide has an average MW up to about 100,000 Da.
[39] The present invention also provides the formulation of any one of embodiments [1]-[38], wherein in the unit of formula (I), y is up to about 5,000.
[40] The present invention also provides the formulation of any one of embodiments [1]-[38], wherein in the unit of formula (I), y is up to about 4,000.
[41] The present invention also provides the formulation of any one of embodiments [1]-[38], wherein in the unit of formula (I), y is between about 10 and about 5,000.
[42] The present invention also provides the formulation of any one of embodiments [1]-[41], wherein the one or more monosaccharide units (M) comprise glucopyranose units and the weight ratio of glucopyranose units to pendent groups is about 1:1 to about 100:1.
[43] The present invention also provides the formulation of any one of embodiments [1]-[42], wherein the polysaccharide is a natural polysaccharide (PS).
[44] The present invention also provides the formulation of any one of embodiments [1]-[43], wherein the polysaccharide is linear.
[45] The present invention also provides the formulation of any one of embodiments [1]-[43], wherein the polysaccharide is branched.
[46] The present invention also provides the formulation of any one of embodiments [1]-[45], wherein the polysaccharide comprising the unit of formula (I) is a compound of formula (II):

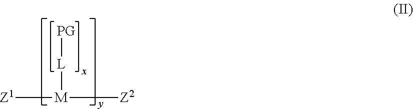

wherein,

each M is a monosaccharide unit;

each L is a suitable linking group, or is a direct bond;

each PG is a pendent group;

each x is independently 0 to about 3, such that when x is 0, the bond between L and M is absent;

y is about 3 to about 5,000; and

Z¹ and Z² are each independently hydrogen, OR¹, OC(═O)R¹, CH₂OR¹, SiR¹ or CH₂OC(═O)R¹;

wherein each R¹ is independently hydrogen, alkyl, cycloalkyl, cycloalkyl alkyl, aryl, aryl alkyl, heterocyclyl or heteroaryl,

wherein each alkyl, cycloalkyl, aryl, heterocycle and heteroaryl is optionally substituted, and

wherein each alkyl, cycloalkyl and heterocycle is optionally partially unsaturated.
[47] The present invention also provides the formulation of any one of embodiments [1]-[46], wherein the one or more pendent groups provide a degree of substitution in the range of about 0.5-2.
[48] The present invention also provides the formulation of any one of embodiments [1]-[47], wherein the one or more pendent groups are the same.
[49] The present invention also provides the formulation of any one of embodiments [1]-[47], wherein the one or more pendent groups are different.
[50] The present invention also provides the formulation of any one of embodiments [1]-[49], wherein the pendent group is linked to the glucopyranose unit via a metabolically cleavable covalent bond.
[51] The present invention also provides the formulation of any one of embodiments [1]-[49], wherein the pendent group is linked to the glucopyranose unit via a metabolically cleavable carboxylic ester bond.
[52] The present invention also provides the formulation of any one of embodiments [1]-[49], wherein the pendent group is linked to the glucopyranose unit via a metabolically cleavable carboxylic ester, diester, carbonate, borate, or silyl ether.
[53] The present invention also provides the formulation of any one of embodiments [1]-[52], wherein the one or more of the pendent groups comprise an active pharmaceutical ingredient (API).
[54] The present invention also provides the formulation of any one of embodiments [1]-[53], wherein the one or more pendent groups comprise a linear, straight-chain or branched C₁-C₂₀ alkyl group; an amine terminated pendant group; or a hydroxyl terminated pendant group.
[55] The present invention also provides the formulation of any one of embodiments [1]-[54], wherein the API has a solubility of less than about 250 mg/L in the biocompatible solvent system, at 25° C. and 1 atm.
[56] The present invention also provides the formulation of any one of embodiments [1]-[54], wherein the API has a solubility of less than about 100 mg/L in the biocompatible solvent system, at 25° C. and 1 atm.
[57] The present invention also provides the formulation of any one of embodiments [1]-[54], wherein the API has a solubility of less than about 50 mg/L in the biocompatible solvent system, at 25° C. and 1 atm.
[58] The present invention also provides the formulation of any one of embodiments [1]-[57], wherein the API is hydrophilic.
[59] The present invention also provides the formulation of any one of embodiments [1]-[58], wherein the API has a water solubility of greater than about 25 g/L, at 25° C. and 1 atm.
[60] The present invention also provides the formulation of any one of embodiments [1]-[58], wherein the API has a water solubility of greater than about 100 g/L, at 25° C. and 1 atm.
[61] The present invention also provides the formulation of any one of embodiments [1]-[60], wherein the API is a macromolecule, a protein, a peptide, a gene, a polynucleotide or analog thereof, a nucleotide, a biological agent, a small molecule, or a complex thereof.
[62] The present invention also provides the formulation of any one of embodiments [1]-[61], wherein the API is suspended in the formulation.
[63] The present invention also provides the formulation of any one of embodiments [1]-[62], wherein the API is present in about 0.1 wt. % to about 30 wt. % of the formulation.
[64] The present invention also provides the formulation of any one of embodiments [1]-[63], wherein the API is a PEGylated protein, PEGylated aptamer, enzyme, blood clotting factor, cytokine, hormone, growth factor, antibody or siRNA.
[65] The present invention also provides the formulation of any one of embodiments [1]-[64], wherein the API is in the form of a spray-dried protein.
[66] The present invention also provides the formulation of any one of embodiments [1]-[65], that does not further comprise an API that is substantially soluble in the biocompatible solvent system.
[67] The present invention also provides the formulation of any one of embodiments [1]-[66], that is a gel.
[68] The present invention also provides the formulation of any one of embodiments [1]-[67], that is flowable.
[69] The present invention also provides the formulation of any one of embodiments [1]-[68], that is suitable for forming an implant in vivo.
[70] The present invention also provides the formulation of any one of embodiments [1]-[68], that is suitable for forming a controlled-release implant in vivo.
[71] The present invention also provides the formulation of any one of embodiments [1]-[68], that is formulated as an injectable delivery system.
[72] The present invention also provides the formulation of any one of embodiments [1]-[71], that is formulated as an injectable ocular delivery system.
[73] The present invention also provides the formulation of any one of embodiments [1]-[72], that is formulated as an injectable subcutaneous delivery system.
[74] The present invention also provides the formulation of any one of embodiments [1]-[71], that is formulated as an injectable parenteral delivery system.
[75] The present invention also provides the formulation of any one of embodiments [1]-[74], that is formulated for injection through a 25 gauge needle, or a higher gauge needle.
[76] The present invention also provides the formulation of any one of embodiments [1]-[75], having a volume of about 10 μL to about 100 μL.
[77] The present invention also provides the formulation of any one of embodiments [1]-[76], having a volume of about 0.01 mL to about 2.0 mL.
[78] The present invention also provides the formulation of any one of embodiments [1]-[77], that is a homogenous suspension.
[79] The present invention also provides the formulation of any one of embodiments [1]-[78], that is chemically and physically stable for up to about 2 years.
[80] The present invention also provides the formulation of any one of embodiments [1]-[79], that has a viscosity less than about 5000 cP at 37° C.
[81] The present invention also provides the formulation of any one of embodiments [1]-[80], that is a suspension of APIs having an average particle size of less than about 20 microns.
[82] The present invention also provides the formulation of any one of embodiments [1]-[81], wherein there is little or no chemical interaction between each of the biocompatible solvent system, biodegradable polymer and active pharmaceutical ingredient (API).
[83] A composition comprising:

(a) a solvent system comprising one or more of ethyl heptanoate, glycofural, benzyl benzoate, glycerol tributyrate and dimethyl isosorbide;

(b) a substituted maltodextrin having an average MW of about 50 kDa to about 350 kDa; the substituted maltodextrin comprising a plurality of (C₂-C₇)alkanoate pendant groups; the substituted maltodextrin having a degree of substitution of about 0.5 to about 2; the substituted maltodextrin having a solubility of at least about 50 g/L in the solvent system at 25° C. and 1 atm; and

(c) an active pharmaceutical ingredient comprising one or more of a PEGylated protein, PEGylated aptamer, enzyme, blood clotting factor, cytokine, hormone, a growth factor, an antibody and siRNA;

wherein the solubility of the active pharmaceutical ingredient is less than about 250 mg/L in the solvent system at 25° C. and 1 atm and the solubility of the active pharmaceutical ingredient is greater than about 25 g/L in water at 25° C. and 1 atm;

wherein the active pharmaceutical ingredient is suspended in the formulation and is present in about 0.1 wt. % to about 30 wt. % of the formulation.
[84] A method comprising administering to a mammal an effective amount of a formulation comprising:

(a) a biocompatible solvent system;

(b) a biodegradable polymer that is substantially soluble in the biocompatible solvent system, the biodegradable polymer comprising a polysaccharide comprising a unit of formula (I):

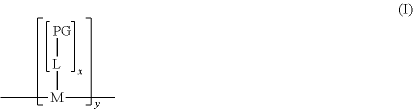

wherein,

each M is independently a monosaccharide unit;

each L is independently a suitable linking group or a direct bond;

each PG is independently a pendent group;

each x is independently 0 to about 3, such that when x is 0, the bond between L and M is absent;

y is 3 to about 10,000; and

(c) an active pharmaceutical ingredient (API) that is substantially insoluble in the biocompatible solvent system.
[85] The present invention also provides the method of embodiment [84], wherein subsequent to the administration, an implant is formed in vivo in the mammal.
[86] The present invention also provides the method of any one of embodiments [84]-[85], wherein subsequent to the administration, an essentially homogeneous implant is formed in vivo in the mammal.
[87] The present invention also provides the method of any one of embodiments [84]-[86], wherein the API is locally delivered.
[88] The present invention also provides the method of any one of embodiments [84]-[86], wherein the API is systemically delivered.
[89] The present invention also provides the method of any one of embodiments [84]-[88], wherein the formulation is administered as an injectable formulation via an ocular administration.
[90] The present invention also provides the method of any one of embodiments [84]-[88], wherein the formulation is administered subcutaneously.
[91] The present invention also provides the method of any one of embodiments [84]-[90], wherein a solid biodegradable implant is formed in vivo and releases an effective amount of the API as the solid implant biodegrades in the mammal.
[92] The present invention also provides the method of any one of embodiments [84]-[91], wherein a solid biodegradable implant is formed in vivo and releases an effective amount of the API by diffusion, erosion, absorption, degradation, or a combination thereof, as the solid implant biodegrades in the mammal.
[93] The present invention also provides the method of any one of embodiments [84]-[92], that effectively treats at least one of the following diseases or disorders: age-related macular degeneration (wet and dry), diabetic macular edema (DME), glaucoma, keratoconjunctivitis sicca (KCS) or dry eye syndrome, multiple sclerosis, rheumatoid arthritis, Alzheimer's disease, Parkinson's disease, amyotrophic lateral sclerosis (ALS), Hepatitis B and C, and systemic lupus erythematosus.
[94] The present invention also provides the method of any one of embodiments [84]-[93], wherein the formulation is administered in vivo through a needle.
[95] The present invention also provides the method of any one of embodiments [84]-[94], that delivers an effective amount of API in a sustained, zero-order release profile.
[96] The present invention also provides the method of any one of embodiments [84]-[95], wherein the formulation is administered to the mammal about once a day to about once per 6 months.
[97] The present invention also provides the method of any one of embodiments [84]-[96], wherein the formulation has a volume of about 10 μL to about 100 μL.
[98] The present invention also provides the method of any one of embodiments [84]-[96], wherein the formulation has a volume of about 0.01 mL to about 2.0 mL.
[99] The present invention also provides the method of any one of embodiments [84]-[98], wherein a solid biodegradable implant is formed in vivo and biodegrades within about 1 year after the formulation is administered.
[100] The present invention also provides the method of any one of embodiments [84]-[99], wherein a solid biodegradable implant is formed in vivo, with little or no initial burst of the active pharmaceutical ingredient (API).
[101] The present invention also provides the method of any one of embodiments [84]-[100], wherein a solid biodegradable implant is formed in vivo and is substantially monolithic, such that the API is substantially uniformly dispersed throughout the solid biodegradable implant.
[102] The present invention also provides the formulation of any one of embodiments [1]-[82], or the composition of embodiment [83], for the treatment of a disease.
[103] The present invention also provides the formulation of any one of embodiments [1]-[82], or the composition of embodiment [83], for the treatment of at least one of the following diseases or disorders: age-related macular degeneration (wet and dry), diabetic macular edema (DME), glaucoma, keratoconjunctivitis sicca (KCS) or dry eye syndrome, multiple sclerosis, rheumatoid arthritis, Alzheimer's disease, Parkinson's disease, amyotrophic lateral sclerosis (ALS), Hepatitis B and C, and systemic lupus erythematosus.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

Table 3 provides an identification of the specific polymers used in the examples below. The abbreviations “Glu2”, “Glu6D”, and “MO40” refer to maltodextrin polymers having an approximate molecular weight as shown in the table. The abbreviations “Hex” and “Pro” refer to hexanoate and propanoate pendant groups on the maltodextrin polymers. The number after “Hex” and “Pro” refers to the degree of substitution on the polymers.

TABLE 3
Index of Biodegradable Polysaccharides Polymers

| Designation | Maltodextrin M_{w} | Pendent Hydrophobic Group |
|---|---|---|
| Glu2-Hex-x | 330 kDa | Hex = hexanoate |
| Glu2-Pro-x | 330 kDa | Pro = Propanoate |
| Glu6D-Hex-x | 150 kDa | Hex = hexanoate |
| MO40-Hex-x | 50 kDa | Hex = hexanoate |

x = degree of substitution (DS);
final MW of polymer depends on DS.

### Example 1

### The Effect of Polymer Addition to Solvent

Maltodextrin-Glu-2 modified with hexanoic acid at a degree of substitution of 1.7 (Glu2-Hex-1.7) was dissolved at 25 mg/mL in benzyl benzoate. To two microcentrifuge tubes, 12 mg spray-dried horseradish peroxidase (HRP) microparticles (˜5 μm, 70 wt. % HRP, 30 wt. % trehalose) were added. Benzyl benzoate was dispensed (200 μL) to one tube and 200 μL of 25 mg/mL Glu2-Hex-1.7 to the other. The mixtures were vortexed and sonicated to obtain a homogenous suspension of spray-dried HRP particles.

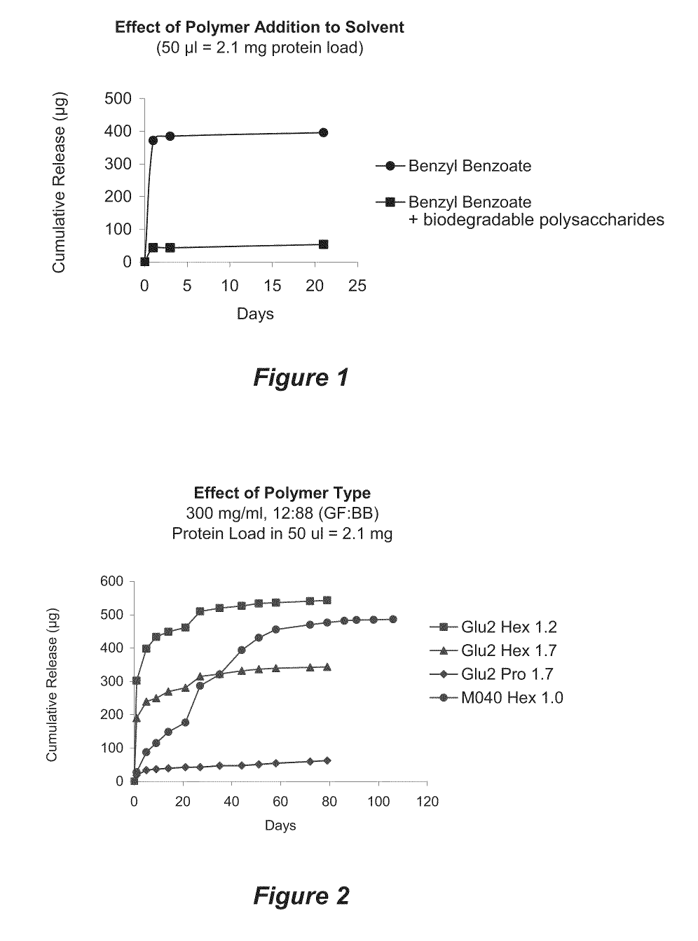

Each formulation (50 μL) was dispensed to microcentrifuge tubes containing 0.5 mL phosphate-buffered saline (PBS, pH 7.4) and incubated static at 37° C. At specific time intervals, eluents were removed and fresh PBS was added. HRP concentration in the eluents was determined by measuring absorbance at 403 nm and using the 1% solution extinction coefficient for HRP (A403_{1%}=15). The addition of polymer to benzyl benzoate reduced the initial burst release of HRP (FIG. 1).

### Example 2

### The Effect of Polymer Type

Formulations were prepared with polymers containing different MW maltodextrins, different degrees of substitution with respect to alkanoate group and alkanoate chain length. All polymer solutions were prepared at 300 mg/mL in a solvent ratio of 12% v/v glycofurol and 88% v/v benzyl benzoate. Solvents and polymer were heated in a 55° C. incubator for 15 minutes and then vortexed to speed the dissolution of polymer. To microcentrifuge tubes, 12 mg of spray-dried rabbit Fab particles (˜5 μm, 70 wt. % Fab, 30 wt. % trehalose) were added. Polymer solutions (0.2 mL) were added to tubes resulting in 60 mg protein particles per mL of polymer solution (4.1 wt. % protein particles). Formulations were vortexed, sonicated, and mixed with a positive displacement pipette to insure a homogenous suspension. Using standard pipettes, 50 μL of each formulation was dispensed into microcentrifuge tubes and 1 mL PBS was gently added. At specific time intervals, eluents were removed and fresh PBS was added.

Fab concentrations of the eluents were determined by performing a tryptophan fluorescence assay. See for example, the techniques described by T. E. Creighton in Proteins: Structures and Molecular Properties, 2^{nd} Ed., W.H. Freeman and Company, 1993. In a 96-well microtiter black plate, 100 μL of eluent samples and a set of serially diluted standards of Fab were added. To all wells, 100 μL 12 N guanidine HCl in deionized water was added. The plate was kept at −20° C. for 10 minutes and fluorescence was measured using a fluorescence microplate reader (λₑₓ=290 nm, λₑₘ=370 nm). The concentration of Fab in the eluent samples was determined by interpolating fluorescence units from the standard curve. Different polymer types produced different elution profiles; see FIG. 2.

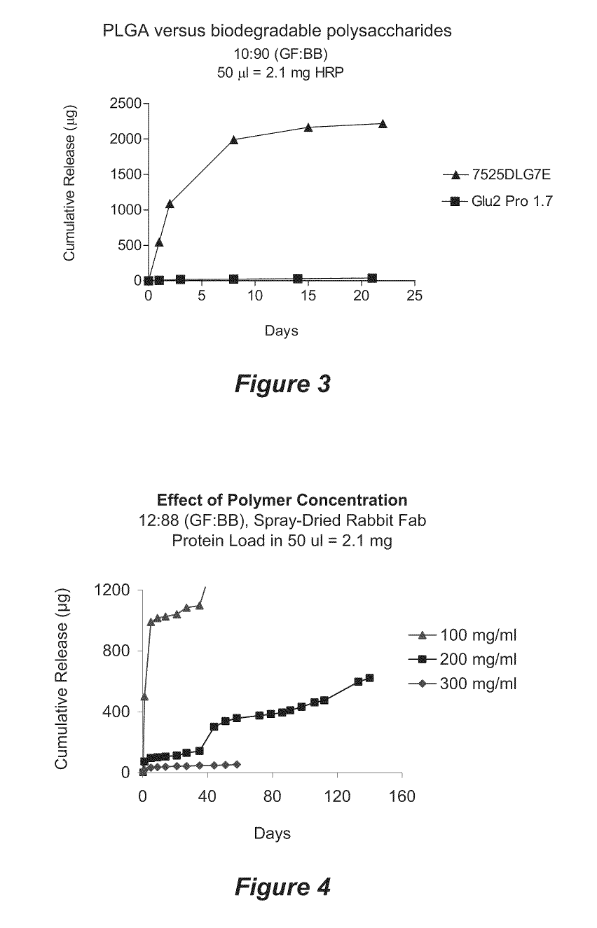

A standard poly(DL-lactide-co-glycolide) formulation was also evaluated and compared with a Glu2-Pro-1.7 formulation. A polymer comprising of 75 mole % DL-lactide, 25 mole % glycolide (7525DLG7E, IV Spec: 0.6-0.8, end group: ester) was first dissolved at 300 mg/mL in a solvent ratio of 10% v/v glycofurol and 90% v/v benzyl benzoate. Spray-dried HRP particles (4.1 wt. %) were then added and mixed thoroughly by vortexing and sonication. To a microcentrifuge tube, 50 μL of the formulation was dispensed and 1 mL PBS was added to begin static elution at 37° C. Elutions were performed and HRP concentrations were measured as described in Example 1. Greater elution control was observed with biodegradable polysaccharides polymer compared to a standard poly(DL-lactide-co-glycolide) formulation (see FIG. 3).

### Example 3

### The Effect of Polymer Concentration

Three different concentrations of Glu2-Pro-1.7 (100, 200, and 300 mg/mL) were prepared at a solvent ratio of 12% v/v glycofurol and 88% v/v benzyl benzoate. Elutions were performed and Fab concentrations of eluents were determined as described in Example 2. Loss of elution control was observed at 100 mg/mL polymer (FIG. 4).

### Example 4

### The Effect of Solvent

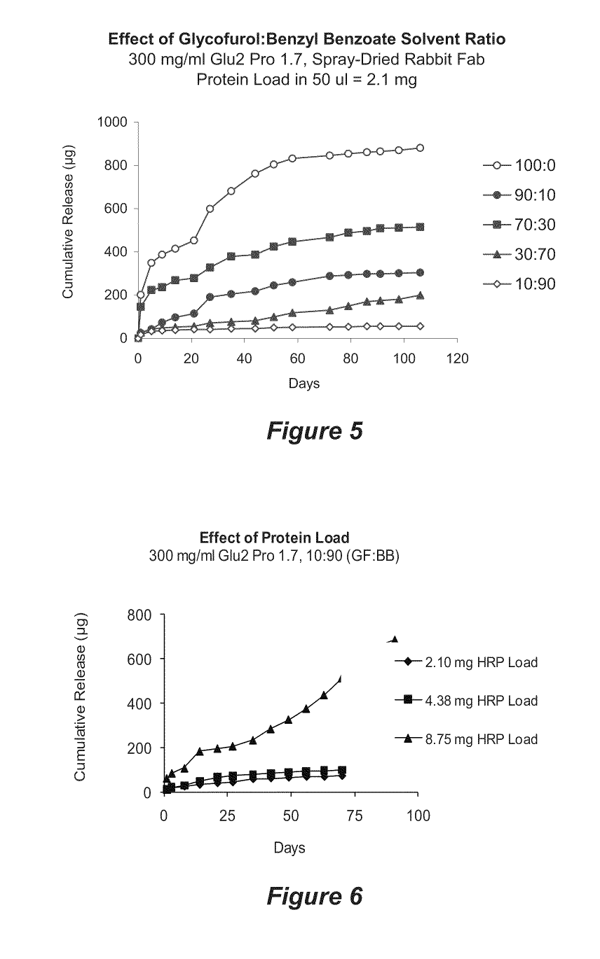

Formulations were prepared with different ratios of glycofurol and benzyl benzoate. Maltodextrin-Glu-2 modified with propanoic acid at a degree of substitution of 1.7 (Glu2-Pro-1.7) was dissolved at 300 mg/mL in various ratios of solvent. To microcentrifuge tubes, 12 mg of spray-dried rabbit Fab particles (˜5 μm, 70 wt. % Fab, 30 wt. % trehalose) were added. Polymer solutions (0.2 mL) were added to tubes resulting in 60 mg protein particles per mL of polymer solution. The Fab particles were suspended by vortexing and sonication. Elutions were performed and Fab concentrations of eluents were determined as in Example 2. The elution was found to be tunable by adjusting the solvent ratio (FIG. 5).

### Example 5

### The Effect of Protein Load

Glu2-Pro-1.7 at 300 mg/mL was dissolved in a solvent ratio of 10% v/v glycofurol and 90% v/v benzyl benzoate. Three formulations were prepared with different loads of spray-dried HRP particles: 4.1 wt. %, 8.1 wt. %, and 15 wt. %. Using a positive displacement pipette, 50 μL of each formulation was dispensed to the tubes. One mL PBS was slowly added to each tube. Each tube was then incubated staticly at 37° C. Elutions were performed and Fab concentrations of eluents were determined as in Example 1. Low burst and controlled elution at 15 wt. % protein particles was observed (FIG. 6).

### Example 6

### Fab Release from Formulations

Maltodextrin-Glu-2 substituted with hexanoate pendent groups to degree of substitution of 1.6 (“G2-hex-1.6”) was dissolved in either benzyl benzoate (BB) or ethylheptanoate (EH) at 300 mg/mL at ˜60° C. The viscosity of the solution increased upon cooling. The benzylbenzoate solution remained a viscous solution, whereas the ethylheptanoate solution formed a gel over the course of 12 hours. A similar observation was made for a less viscous 200 mg/mL solution of G2-hex-1.6 in EH. The gel redissolved upon heating to 55° C.

Fab fragments of non-specific rabbit IgG was spray-dried to form particles having average particles sizes of about 5 μm. The particles contained 70% protein and 30% trehalose. Thirty three μL of freshly cooled 300 mg/mL solution (10 mg of polymer) was aliquotted into a vial containing 2.9 mg of Fab particles. The Fab particles were then mixed into the solution. The mixture was left at room temperature (˜23° C.) over night (˜12 hours), during which time the mixture turned into a gel.

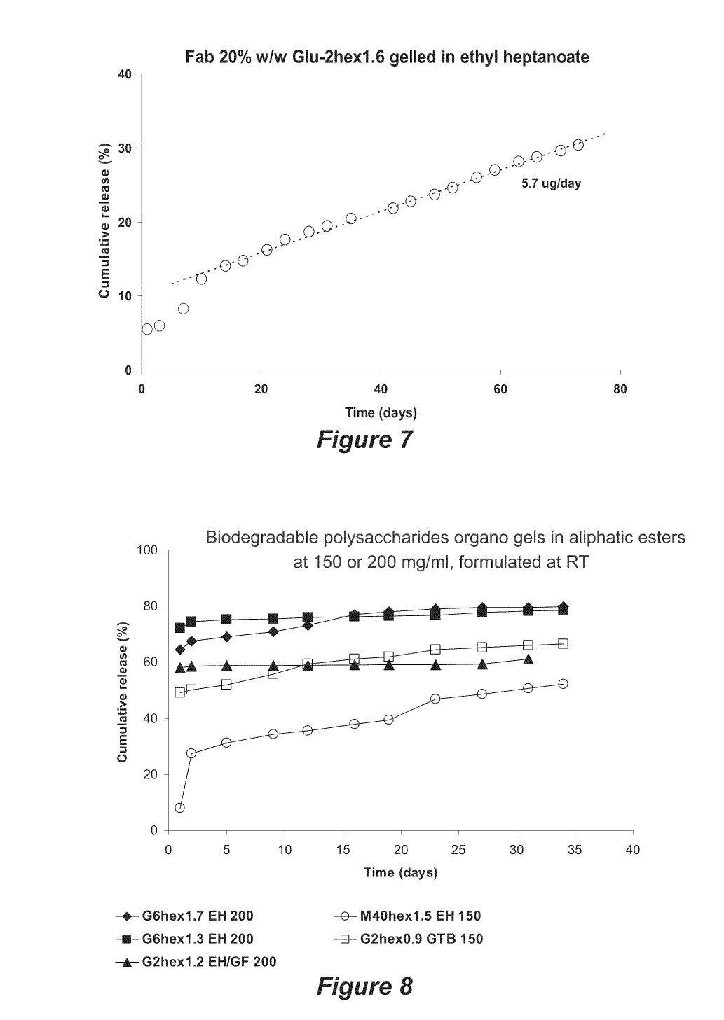

The gel was placed in 1 mL of PBS at 37° C. At specific time intervals the buffer was replaced. Fab concentrations were determined using tryptophan fluorescence: to a 100 μL sample, 100 μL 12 N guanidine.HCl solution in DDW was added. The sample was cooled to −20° C. for 10 minutes and the fluorescence measurements were recorded (λₑₓ=290 nm, λₑₘ=370 nm), as illustrated in FIG. 7.

### Example 7

### Solubility and Subsequent Gel-Forming Behavior

Materials. Solubility and subsequent gel-forming behavior was investigated using different maltodextrin starting materials (Glu-2, Glu-6D, or MO40), two different grafts: hexanoate and propanoate, at various degrees of substitution. The following solvents were used, categorized according to typical solvent classes:
a. Aromatic ester: benzyl benzoate (BB)
b. Aliphatic esters: ethylheptanoate (EH), ethyloctanoate (EO)
c. Glycerides: glyceroltriacetate (Triacetin), glyceroltributyrate (GTB)

Methods. Polymers were weighed out and placed in different solvents such that initial formulations were aimed at 300 mg/mL polymer concentration. A 150 mg/mL or 90% solvent/10% glycofurol (GF) or a combination of both was used in those combinations were no complete solution was obtained or the resulting solution/gel was too viscous even at elevated temperatures (55° C.) to further formulate with protein particles.

Results.

TABLE 7-1
Ethyl heptanoate.

| | Glu2- | Glu2-hex- | Glu2- | Glu6- | Glu6- | MO40- | MO40- |
|---|---|---|---|---|---|---|---|
| Solvent | hex-0.5 | 0.9 | hex1.6 | hex-1.3 | hex-1.7 | hex-1.5 | hex-1.7 |
| EH | | Gels at RT | | Not | Not | Viscous | Not |
| 150 mg/mL | | white gel ¶ | | viscous § | viscous § | Slightly | viscous § |
| | | | | | | haizy § | |
| EH | Did not | Partially | Less viscous | Viscous § | Viscous § | viscous § | Viscous § |
| 200 mg/mL | Dissolve £ | dissolved ¥ | solution. | | | | |
| | | | gel ¶ | | | | |
| EH | Did not | Partially | Viscous | | | | |
| 300 mg/mL | Dissolve £ | dissolved ¥ | solution | | | | |
| | | | Gel ¶ | | | | |

¶: Gelled over time.
§: Polymer dissolved, no gel formation over time.
¥: Polymers partially dissolved or gelled at 55° C.
£: Polymers did not dissolve.

TABLE 7-2
Comparison of EH, BB, and EO, in combination with glycofurol.

| Solvent | MO40-Pro- | MO40- | Glu2-pro- | Glu2- | MO40-hex- | |
|---|---|---|---|---|---|---|
| 90%/10% | 2.2 | pro-2.8 | 1.7 | pro-2.7 | 1.0 | Glu2-hex-1.2 |
| EH/GF | 300 mg/mL | 300 mg/mL | Did not | 300 mg/mL | 300 mg/mL | 200 mg/mL |
| | Diss at 55° C. | Not | dissolve £ | Viscous § | Slightly viscous | Slightly haizy, |
| | with GF | viscous § | | | soln § | viscous soln § |
| | dense gel [symbol] | | | | | |
| BB/GF | 300 mg/mL | 300 mg/mL | 300 mg/mL | 300 mg/mL | | |
| | Viscous/clear § | Not very | At 55° C. | Very | | |
| | | viscous § | Visc soln/ | viscous | | |
| | | | thick gel at | solution § | | |
| | | | RT | | | |
| EO/GF | partially | 300 mg/mL | Did not | 300 mg/mL | | |
| | dissolved | Not | dissolve £ | Viscous/ | | |
| | crashed out as | viscous/ | | clear | | |
| | dense gel ¥ | clear | | solution § | | |
| | | solution § | | | | |

[symbol]

: Gelled upon cooling.
¶: Gelled over time.
§: Polymer dissolved, no gel formation over time.
¥: Polymers partially dissolved or gelled at 55° C.
£: Polymers did not dissolve.

TABLE 7-3
Triglycerides.

| Solvent | MO40-hex-1.0 | MO40-hex-1.7 | Glu2-hex-0.9 | Glu2-hex-1.6 |
|---|---|---|---|---|
| Glycerol | 300 mg/mL; | 300 mg/mL; | 100 mg/mL; | 300 mg/mL; |
| Triacetate | Dissolved at 55° C. | Dissolved at 55° C. | Dissolves at 55° C. | Dissolved at 55° C. |
| (triacetin) | Gelled fast at RT. | Gelled at RT [symbol] | Gelled fast at RT. | Gelled at RT [symbol] |
| | White gel | | White gel [symbol] | |
| | Similar result seen at | | | |
| | 100 mg/ml [symbol] | | | |
| Glycerol | 300 mg/mL; | 300 mg/mL (no | 150 mg/mL; | GTB/GF: visc. |
| Tributyrate | Dissolved with 10% | GF); | at 55° C.: haizy | liquid 150 mg/mL |
| (GTB) | GF; | Viscous; | soln. Dissolved | Gel¶ |
| (usually with GF) | Gel¶ | Gel¶ | by adding 10% | |
| | | | GF§ | |

[symbol]

Gelled upon cooling.
¶Gelled over time.
§Polymer dissolved, no gel formation over time.
¥Polymers partially dissolved or gelled at 55° C.
£Polymers did not dissolve.

### Example 8

### Fab Release from Different Formulations, Prepared at Room Temperature

Spray-dried Fab (2.9 mg; 2 mg protein) was combined with 40 μL of the various prepared formulations as indicated in Tables 7-1, 7-2, and 7-3 of Example 7, and was mixed by hand in a microcentrifuge tube. Formulations that did not gel at room temperature (indicated by ¶ or §) were added to protein at room temperature. After obtaining a homogeneous mixture, the formulation was either injected into PBS, or PBS was added onto the formulation.

FIG. 8 illustrates cumulative release profiles of Fab from biodegradable polysaccharides organogels in aliphatic esters at 150 or 200 mg/mL, formulated at room temperature, wherein the organogel formulation is injected into PBS solution.

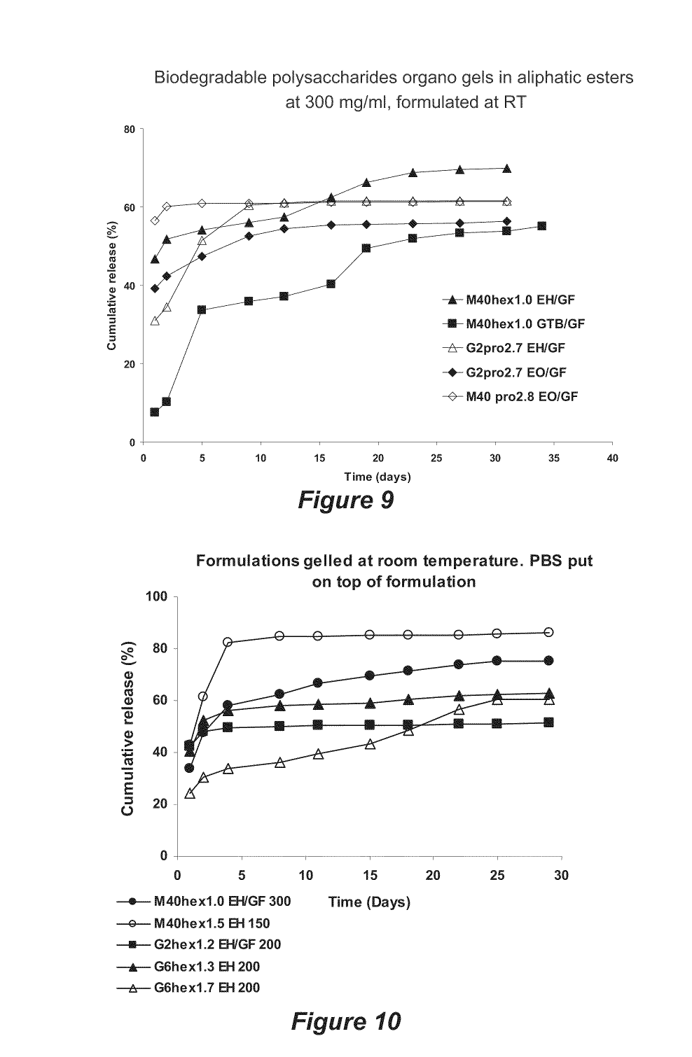

FIG. 9 illustrates cumulative release profiles of Fab from biodegradable polysaccharides organogels in aliphatic esters at 300 mg/mL, wherein the organogel formulation is injected into a PBS solution.

FIG. 10 illustrates cumulative release profiles of Fab from biodegradable polysaccharides organogels, in ethyl hexanoate at 300 mg/mL, wherein a PBS solution is put on top of the organogel formulation.

### Example 9

### Fab Release from Different Formulations, Prepared at Room Temperature

Formulations marked with [symbol] (in Tables 7-1, 7-2, and 7-3; Example 7) were heated in an oven at 55° C. and added to the protein particles immediately upon removal from the oven. Spray-dried Fab (2.9 mg; 2 mg protein) was combined with 40 μL of the various prepared formulations as indicated in Tables 7-1,7-2 and 7-3, and was mixed by hand in a microcentrifuge tube. Upon cooling 1 mL of PBS was added onto the formulation.

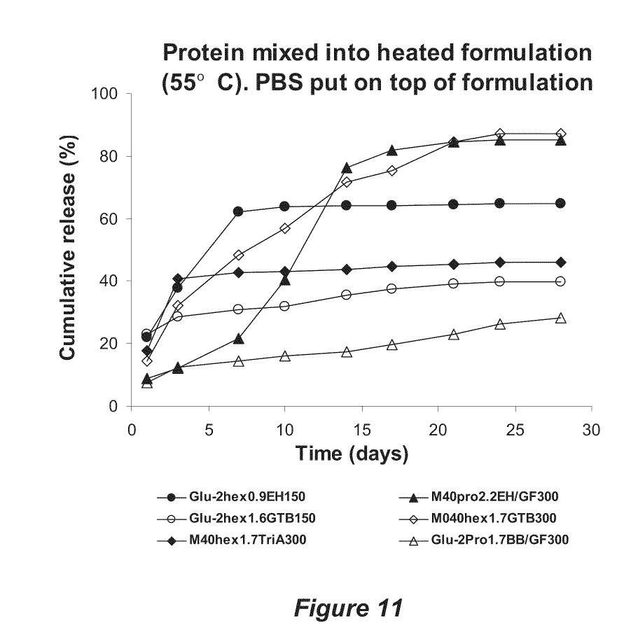

FIG. 11 illustrates cumulative release profiles of Fab from biodegradable polysaccharides organogels formulated at 55° C. in various solvents at concentrations of 150-300 mg/mL, wherein the polymer solution was heated and added to Fab particles; the formulation was then mixed and allowed to cool, followed by adding PBS to the formulation.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A formulation comprising:
(a) a biocompatible solvent system;
(b) a biodegradable polymer that is substantially soluble in the biocompatible solvent system, the biodegradable polymer comprising a polysaccharide comprising a unit of formula (I): 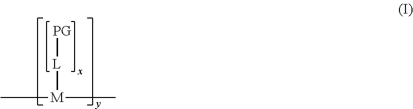
wherein,
each M is independently a monosaccharide unit;
each L is independently a suitable linking group or a direct bond;
each PG is independently a pendent group;
each x is independently 0 to about 3, such that when x is 0, the bond between L and M is absent;
y is 3 to about 10,000; and
(c) an active pharmaceutical ingredient (API) that is substantially insoluble in the biocompatible solvent system.

2. The formulation of claim 1, wherein the solvent system comprises ethyl heptanoate, glycofural, benzyl benzoate, glycerol tributyrate, dimethylisosorbide, or a mixture thereof

3. The formulation of claim 1, wherein the biodegradable polymer is present in about 10 wt. % to about 40 wt. % of the formulation.

4. The formulation of claim 1, wherein the biodegradable polymer is present in about 40 wt. % to about 90 wt. % of the formulation.

5. The formulation of claim 1, wherein the monosaccharide units comprise glucopyranose units.

6. The formulation of claim 1, wherein the monosaccharide units comprise D-glucopyranose.

7. The formulation of claim 1, wherein the monosaccharide units comprise α-D-glucopyranose.

8. The formulation of claim 1, wherein the monosaccharide units comprise glucopyranose units linked by α(1→4) glycosidic bonds.

9. The formulation of claim 1, wherein the monosaccharide units comprise glucopyranose units linked by α(1→6) glycosidic bonds.

10. The formulation of claim 1, wherein the monosaccharide units comprise non-macrocyclic poly-α(1→4)glucopyranose.

11. The formulation of claim 1, wherein the monosaccharide units comprise non-macrocyclic poly-α(1→6) glucopyranose.

12. The formulation of claim 1, wherein the monosaccharide units comprise glucopyranose units and at least about 90% of the glucopyranose units are linked by α(1→4) glycosidic bonds.

13. The formulation of claim 1, wherein the polysaccharide comprises up to about 5,000 glucopyranose units.

14. The formulation of claim 1, wherein the polysaccharide has an average MW up to about 1,000,000 Da.

15. The formulation of claim 1, wherein in the unit of formula (I), y is up to about 5,000.

16. The formulation of claim 1, wherein the one or more monosaccharide units (M) comprise glucopyranose units and the weight ratio of glucopyranose units to pendent groups is about 1:1 to about 100:1.

17. The formulation of claim 1, wherein the polysaccharide comprising the unit of formula (I) is a compound of formula (II): 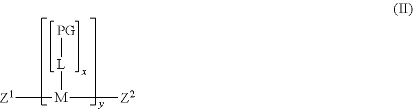
wherein,
each M is a monosaccharide unit;
each L is a suitable linking group, or is a direct bond;
each PG is a pendent group;
each x is independently 0 to about 3, such that when x is 0, the bond between L and M is absent;
y is about 3 to about 5,000; and
Z¹ and Z² are each independently hydrogen, OR¹, OC(═O)R¹, CH₂OR¹, SiR¹ or CH₂OC(═O)R¹;
wherein each R¹ is independently hydrogen, alkyl, cycloalkyl, cycloalkyl alkyl, aryl, aryl alkyl, heterocyclyl or heteroaryl,
wherein each alkyl, cycloalkyl, aryl, heterocycle and heteroaryl is optionally substituted, and
wherein each alkyl, cycloalkyl and heterocycle is optionally partially unsaturated.

18. The formulation of claim 1, wherein the one or more pendent groups provide a degree of substitution in the range of about 0.5-2.

19. The formulation of claim 1, wherein the pendent group is linked to the glucopyranose unit via a metabolically cleavable carboxylic ester, diester, carbonate, borate, or silyl ether.

20. The formulation of claim 1, wherein the one or more of the pendent groups comprise an active pharmaceutical ingredient (API).

21. The formulation of claim 1, wherein the one or more pendent groups comprise a linear, straight-chain or branched C₁-C₂₀ alkyl group; an amine terminated pendant group; or a hydroxyl terminated pendant group.

22. The formulation of claim 1, wherein the API is a macromolecule, a protein, a peptide, a gene, a polynucleotide or analog thereof, a nucleotide, a biological agent, a small molecule, or a complex thereof

23. The formulation of claim 1, wherein the API is present in about 0.1 wt. % to about 30 wt. % of the formulation.

24. The formulation of claim 1, wherein the API is a PEGylated protein, PEGylated aptamer, enzyme, blood clotting factor, cytokine, hormone, growth factor, antibody or siRNA.

25. A composition comprising:
(a) a solvent system comprising one or more of ethyl heptanoate, glycofural, benzyl benzoate, glycerol tributyrate and dimethyl isosorbide;
(b) a substituted maltodextrin having an average MW of about 50 kDa to about 350 kDa; the substituted maltodextrin comprising a plurality of (C₂-C₇)alkanoate pendant groups; the substituted maltodextrin having a degree of substitution of about 0.5 to about 2; the substituted maltodextrin having a solubility of at least about 50 g/L in the solvent system at 25° C. and 1 atm; and
(c) an active pharmaceutical ingredient comprising one or more of a PEGylated protein, PEGylated aptamer, enzyme, blood clotting factor, cytokine, hormone, a growth factor, an antibody and siRNA;
wherein the solubility of the active pharmaceutical ingredient is less than about 250 mg/L in the solvent system at 25° C. and 1 atm and the solubility of the active pharmaceutical ingredient is greater than about 25 g/L in water at 25° C. and 1 atm;
wherein the active pharmaceutical ingredient is suspended in the formulation and is present in about 0.1 wt. % to about 30 wt. % of the formulation.

26. A method comprising administering to a mammal an effective amount of an injectable formulation comprising:
(a) a biocompatible solvent system;
(b) a biodegradable polymer that is substantially soluble in the biocompatible solvent system, the biodegradable polymer comprising a polysaccharide comprising a unit of formula (I): 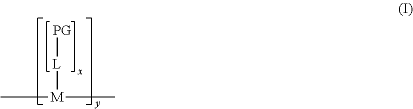
wherein,
each M is independently a monosaccharide unit;
each L is independently a suitable linking group or a direct bond;
each PG is independently a pendent group;
each x is independently 0 to about 3, such that when x is 0, the bond between L and M is absent;
y is 3 to about 10,000; and
(c) an active pharmaceutical ingredient (API) that is substantially insoluble in the biocompatible solvent system.

27. The method of claim 26, wherein subsequent to the administration, an implant is formed in vivo in the mammal.

28. The method of claim 26, that effectively treats at least one of the following diseases or disorders: age-related macular degeneration (wet and dry), diabetic macular edema (DME), glaucoma, keratoconjunctivitis sicca (KCS) or dry eye syndrome, multiple sclerosis, rheumatoid arthritis, Alzheimer's disease, Parkinson's disease, amyotrophic lateral sclerosis (ALS), Hepatitis B and C, and systemic lupus erythematosus.
